(19) United States
(12) Patent Application Publication
(10) Pub. No.: US 2024/0002937 A1
(43) Pub. Date: Jan. 4, 2024

(54) METHOD

(71) Applicant: ULIFETEC SAS, Paris (FR)

(72) Inventors: Ulrich Bockelmann, Paris (FR); Ismail Cissé, Aubervilliers (FR)

(21) Appl. No.: 18/036,439

(22) PCT Filed: Nov. 12, 2021
(86) PCT No.: PCT/EP2021/081578

§ 371 Date: May 11, 2023

(30) Foreign Application Priority Data
Nov. 12, 2020 (GB) 2017861.2

(51) Int. Cl. C12Q 1/6883

(52) U.S. Cl. CPC C12Q 1/6883; C12Q 2600/156

(57) ABSTRACT

The present invention relates to methods of detecting and/or quantifying a RNA, particularly a tRNA, using padlock probes comprising terminal regions complementary to said RNA. The present invention also relates to methods of detecting, diagnosing and/or assessing the clinical severity of an RNA-associated disease.

### FIELD OF THE INVENTION

The present invention relates to methods of detecting and/or quantifying a RNA, particularly a tRNA, using padlock probes comprising terminal regions complementary to said RNA. The present invention also relates to methods of detecting, diagnosing and/or assessing the clinical severity of an RNA-associated disease.

### BACKGROUND TO THE INVENTION

RNA and RNA-protein complexes (RNAPs) are interesting targets for molecular diagnostics. However, it can be technically difficult to specifically quantify an RNA molecule by the commonly used reverse-transcription polymerase chain reaction (RT-PCR) or RNA sequencing (RNAseq) techniques. For instance, the reverse-transcription of the RNA to complementary DNA (cDNA) or the subsequent PCR amplification of the cDNA can be difficult if the RNA (or the cDNA) exhibits stable structural features that prevent specific binding of oligonucleotide primers to the region of interest, or prevent progression of the reverse transcriptase or the DNA polymerase. Chemical modifications can hinder RT-PCR, and the proteins of RNAPs can induce structures in the nucleic acids or stabilize pre-existing structure, and thus prevent processive action of the essential enzymes.

Due to these difficulties, RNAs and RNAPs are sometimes treated as a secondary choice in molecular diagnostics; if possible the DNA molecule is usually preferred. Moreover, DNA is more stable than RNA and reverse-transcription is not required if DNA is used as molecular target. Due to the RNA fragility, RNA samples extracted from medical samples often comes in the form of short fragments, exhibiting a distribution of short sizes, which makes analysis difficult. Short sizes are particularly common in urine, which otherwise is an attractive tissue for diagnostics since it is obtained in a fully non-invasive manner.

One type of RNA, transfer RNA (tRNA) has received increasing attention recently (S. Kirchner, Z. Ignatova, Nat. Rev. Genet. 16, 98-112 (2015)). A method to specifically quantify tRNAs will have applications in research and development in biology, biotechnology and medicine, and in molecular diagnostics. However, tRNA is particularly short, strongly structured and contains post-transcriptional modifications.

Human mitochondrial tRNA (mt-tRNA) is a type tRNA of particular interest. Many mutations in mitochondrial DNA transcribe into mutations in mt-tRNA and are linked to defects in oxidative energy metabolism. It is now emerging that these mutations are also linked to other complex traits, including neurodegenerative diseases, ageing and cancer. MELAS (Mitochondrial Encephalopathy, Lactic Acidosis and Stroke-like episodes) and MERRF (Myoclonic Epilepsy with Ragged Red Fibers) are two classic diseases associated with mitochondrial tRNA mutations in mt-tRNA Leu(UUR) and mt-tRNA Lys, respectively.

MELAS syndrome is a mitochondrial cytopathy associated with mutations of mitochondrial DNA. It can be caused by several mutations, but by far the most frequent one is m.3243A>G, located on the MT-TL1 gene coding for leucine(UUR) mt-tRNA. This mutation was found with a prevalence of 0.14% in the general population of North Cumbria (England); interval of 4-36% with 95% confidence (H. R. Elliott et al, Am. J. Hum. Genet. 83, 254-260 (2008)). It is a heteroplasmic mutation, i.e. mutated and non-mutated mtDNA molecules coexist in the cell. Diseases caused by m.3243A>G represent one of the most frequent groups of OXPHOS deficiencies.

MERRF syndrome is a mitochondrial encephalomyopathy characterized by myoclonic seizures. Four point mutations in the genome can be identified that are associated with MERRF: m.A8344G, m.T8356C, m.G8361A, and m.G8363A. The point mutation m.A8344G is associated with MERRF in 80% of patients (DiMauro, S. and Hirano, M., 2015. Merrf. In GeneReviews. University of Washington, Seattle). This point mutation disrupts the mitochondrial gene for lysine mt-tRNA, which disrupts the synthesis of proteins Thus, there is a demand for methods of detecting and quantifying a RNA, particularly a tRNA.

### SUMMARY OF THE INVENTION

The present inventors have developed a method of detecting and/or quantifying a RNA, particularly a tRNA, using padlock probes comprising terminal regions complementary to said RNA. The present inventors have surprisingly found that the method can be used to reliably detect and quantify the percentage of mutant tRNAs, even though the tRNA is short, strongly structured and exhibits some chemical modifications.

The inventors have also developed a method of detecting, diagnosing and/or assessing the clinical severity of a RNA-associated disease. In particular, the present inventors have surprisingly found that the mutation load of the mt-tRNA can differ significantly from the mt-DNA mutation load. Thus, mt-tRNA mutation load may correlate with the clinical severity of a mitochondrial disease and be used as a prognostic tool and/or as an indicator for the appropriate medical treatment.

In one aspect, the present invention provides a padlock probe for detecting and/or quantifying a RNA. Suitably, the padlock probe comprises terminal regions complementary to the RNA. Suitably, each terminal region is complementary to a region of the RNA.

In some embodiments the RNA is a tRNA, suitably a mitochondrial tRNA (mt-tRNA). Suitable mt-tRNAs include leucine(UUR) mt-tRNA, a lysine mt-tRNA, a methionine mt-tRNA, a tryptophan mt-tRNA, a aspartate mt-tRNA, an isoleucine mt-tRNA, a glycine mt-tRNA, an arginine mt-tRNA, a histidine mt-tRNA, a serine(AGY) mt-tRNA, a leucine(CUN) mt-tRNA, a threonine mt-tRNA, a phenylalanine mt-tRNA, a valine mt-tRNA, a glutamine mt-tRNA, an alanine mt-tRNA, an asparagine mt-tRNA, a cysteine mt-tRNA, a tyrosine mt-tRNA, a serine(UCN) mt-tRNA, a glutamate mt-tRNA, or a proline mt-tRNA. Optionally, the mt-tRNA is a leucine(UUR) mt-tRNA, a lysine mt-tRNA, a histidine mt-tRNA, a leucine(CUN) mt-tRNA, a phenylalanine mt-tRNA, a valine mt-tRNA, a glutamine mt-tRNA, a serine(UCN) mt-tRNA, or a proline mt-tRNA. Optionally, the mt-tRNA is a leucine(UUR) mt-tRNA or a lysine mt-tRNA, optionally wherein the mt-tRNA is a leucine(UUR) mt-tRNA. In some embodiments, the tRNA comprises a 3′ ligated oligonucleotide.

The regions of the RNA to which the terminal regions are complementary may each be 5-30 nucleotides in length, optionally 5-15 nucleotides in length. The terminal regions may be each 5-30 nucleotides in length, optionally 5-15 nucleotides in length. The terminal regions may be in total are 15-50 nucleotides in length, optionally 20-30 nucleotides in length. The terminal regions may be complementary to adjacent regions or non-adjacent regions, optionally wherein the non-adjacent regions are separated by 6 or fewer nucleotides, 5 or fewer nucleotides, 4 or fewer nucleotides, 3 or fewer nucleotides, 2 or fewer nucleotides, or one nucleotide.

The padlock probe may be 50-200 nucleotides in length, 50-150 nucleotides in length, 50-100 nucleotides in length, or 70-100 nucleotides in length. The padlock probe may comprises one or more primer binding sites, optionally wherein the padlock probe comprises a forward primer site and a reverse primer site. The forward primer site may be complementary to a forward primer. The complement of the reverse primer site may be complementary to a reverse primer. Suitably, the padlock probe comprises from 5′ to 3′: a first terminal region; a first primer binding site; a second primer binding site; and a second terminal region, optionally wherein the first primer binding site is a forward primer site and the second primer site is a reverse primer site, or vice versa. The primer binding sites may be 10-30 nucleotides in length. Optionally, the padlock probe comprises a tag sequence.

The padlock probe may be capable of detecting and/or quantifying a pathogenic mutation.

Suitably, a terminal region is complementary to a region of the RNA that is associated with a pathogenic mutation, optionally wherein the pathogenic mutation is a pathogenic single-nucleotide variant. In some embodiments, (i) the RNA is a leucine(UUR) mt-tRNA and the pathogenic mutation is A3243G or T3271C; or (ii) the RNA is a lysine mt-tRNA and the pathogenic mutation is A8344G.

Suitably, a terminal region is complementary to a region of the RNA which comprises a pathogenic mutation site, optionally wherein the pathogenic mutation is a pathogenic single-nucleotide variant. In some embodiments, (i) the RNA is a leucine(UUR) mt-tRNA and the pathogenic mutation site is m.3243 or m.3271; or (ii) the RNA is a lysine mt-tRNA and the pathogenic mutation site is m.8344.

Suitably, a terminal region is complementary to a region of the RNA that comprises a pathogenic mutation, optionally wherein the pathogenic mutation is a pathogenic single-nucleotide variant. In some embodiments, (i) the RNA is a leucine(UUR) mt-tRNA and the pathogenic mutation is A3243G or T3271C; or (ii) the RNA is a lysine mt-tRNA and the pathogenic mutation is A8344G.

In some embodiments, a terminal region is complementary to a region of a nucleotide sequence which comprises or consists of:
(i) a fragment of the nucleotide sequence

(SEQ ID NO: 25)
GUUAAGAUGGCAGGGCCCGGUAAUCGCAUAAAACUUAAAACUUUACAGUC
AGAGGUUCAAUUCCUCUUCUUAACACCA,

optionally wherein the region comprises nucleotide 14 of SEQ ID NO: 25; or
(ii) a fragment of the nucleotide sequence

(SEQ ID NO: 23)
GUUAAGAUGGCAGAGCCCGGUAAUCGCAUAAAACUUAAAACUUUACAGUC
AGAGGUUCAAUUCCUCUUCUUAACACCA,

optionally wherein the region comprises nucleotide 14 of SEQ ID NO: 23.

In some embodiments, a terminal region comprises or consists of the nucleotide sequences TTACCGGGCC or TTACCGGGCT (SEQ ID NOs: 56 and 58), or fragments thereof.

In some embodiments, the terminal regions comprise or consist of the nucleotide sequences CTGCCATCTTAAC and TTACCGGGCC (SEQ ID NOs: 55 and 56) or CTGCCATCTTAAC and TTACCGGGCT (SEQ ID NOs: 57 and 58), or fragments thereof.

In some embodiments, the padlock probe comprises or consists of a nucleotide sequence with at least 70%, at least 80%, at least 85%, at least 90%, at least 95%, at least 98%, or at least 99% sequence identity to

(SEQ ID NO: 63)
CTGCCATCTTAACTGTAGGGGAGGAGCTGACCACACTTATCATTATCTTC
GCACAGACGTTACCGGGCC
or
(SEQ ID NO: 64)
CTGCCATCTTAACTGGTGAGGTAAAGAACGAAGACTTCTTATCATTATCT
ACGAGCGGACGATTACCGGGCT

or a fragment thereof.

In some embodiments the padlock probe comprises or consists of:

(SEQ ID NO: 63)
CTGCCATCTTAACTGTAGGGGAGGAGCTGACCACACTTATCATTATCTTC
GCACAGACGTTACCGGGCC
or
(SEQ ID NO: 64)
CTGCCATCTTAACTGGTGAGGTAAAGAACGAAGACTTCTTATCATTATCT
ACGAGCGGACGATTACCGGGCT

or a fragment thereof.

The padlock probe may be capable of detecting and/or quantifying a modified nucleotide.

Suitably, a terminal region is complementary to a region which is associated with a modified nucleotide. Aberrant modification of the modified nucleotide may be pathological, optionally wherein the aberrant nucleotide modification is the absence of the nucleotide modification. In some embodiments, (i) the RNA is a leucine(UUR) mt-tRNA and the modified nucleotide is τm⁵U or (ii) the RNA is a lysine mt-tRNA and the modified nucleotide is τm⁵s²U.

Suitably, a terminal region is complementary to a region which comprises an aberrantly modified nucleotide, optionally wherein the aberrant nucleotide modification is the absence of the nucleotide modification and/or is pathological. In some embodiments, (i) the RNA is a leucine(UUR) mt-tRNA and the aberrantly modified nucleotide is the absence of τm⁵U or (ii) the RNA is a lysine mt-tRNA and the aberrantly modified nucleotide is the absence of τm⁵s²U.

In some embodiments, a terminal region is complementary to a region of a nucleotide sequence which comprises or consists of fragment of the nucleotide sequence

(SEQ ID NO: 25)
GUUAAGAUGGCAGGGCCCGGUAAUCGCAUAAAACUUAAAACUUUACAGUC
AGAGGUUCAAUUCCUCUUCUUAACACCA

wherein the fragment comprises nucleotide 36 of SEQ ID NO: 25.

The modified nucleotide may be selected from: N2,N2-dimethyl guanosine (m²₂G), 5-methylcytosine (m⁵C), 7-methylguanosine (m⁷G), 5-methoxycarbonylmethyl-2-thiouridine (mcm⁵s²U), 5-methyl uridine (m⁵U), 1-methylguanosine (m¹G), 5-methoxycarbonylmethyluridine (mcm⁵U), 2-methylthio-N6-threonyl carbamoyladenosine (ms²t⁶A), 5-taurinomethyluridine (τm⁵U), 5-taurinomethyl-2-thiouridine (τm⁵s²U), and 2-thiouridine (s²U). Optionally, the modified nucleotide is 5-taurinomethyluridine (τm⁵U) or 5-taurinomethyl-2-thiouridine (τm⁵s²U).

The padlock probe may be capable of detecting and/or quantifying amino acid charging of a tRNA. Suitably, the RNA is a tRNA comprising a 3′ ligated oligonucleotide and a terminal region is complementary to a region of the tRNA that comprises at least part of the 3′ ligated oligonucleotide.

In another aspect, the present invention also provides a kit or composition comprising one or more padlock probes according to the present invention.

In some embodiments, the one or more padlock probes comprise or consist of:
(i) a first padlock probe comprising terminal regions complementary to an RNA, wherein a terminal region is complementary to a region of the RNA that is associated with a pathogenic mutation and the region does not comprise the pathogenic mutation; and
(ii) a second padlock probe comprising terminal regions complementary to said RNA, wherein a terminal region is complementary to a region of the RNA that comprises the pathogenic mutation.

In some embodiments, the kit or composition further comprises:
(iii) a third padlock probe comprising terminal regions complementary to a gene encoding said RNA, wherein a terminal region is complementary to a region of the gene that is associated with a pathogenic mutation and the region does not comprise the pathogenic mutation; and
(iv) a fourth padlock probe comprising terminal regions complementary to the gene encoding said RNA, wherein a terminal region is complementary to a region of the gene that comprises the pathogenic mutation.

Optionally, the kit or composition further comprises a fifth padlock probe comprising terminal regions complementary to a reference nuclear tRNA, optionally a nuclear methionine tRNA and/or the kit or composition further comprises a sixth padlock probe comprising terminal regions complementary to a reference mt-tRNA, optionally a methionine mt-tRNA.

The kit or composition may comprise any combination of the first, second, third, fourth, fifth, and sixth padlock probes.

The kit or composition may further comprise one or more primers, optionally one forward primer and one reverse primer. The one or more primers may be complementary to one or more primer binding sites on the one or more padlock probes. The kit or composition may further comprise one or more capture probes. The kit or composition may further comprise:
(i) a DNA ligase, optionally a Splint R ligase; and/or
(ii) a DNA ligase buffer, optionally a Splint R ligase buffer.

The kit or composition may further comprise:
(i) an amplification buffer;
(ii) a deoxynucleoside triphosphate (dNTP) mix;
(iii) a DNA polymerase; and/or
(iv) a DNA dye.

In another aspect, the present invention provides a method of detecting a RNA using one or more padlock probes according to the present invention.

The method may comprise:
(a) providing a sample comprising one or more RNAs;
(b) hybridising one or more padlock probes to the one or more RNAs to obtain one or more hybridised padlock probes;
(c) circularising the one or more hybridised padlock probes to obtain one or more circularised padlock probes;
(d) optionally purifying the one or more circularised padlock probes;
(e) amplifying the one or more circularised padlock to obtain amplified padlock probes; and
(f) detecting the amplified padlock probes.

The sample may be an RNA sample. Suitably, the sample is obtained or obtainable from urine, muscle and/or blood. Optionally, the method may further comprise a step of extracting, purifying and/or isolating the sample from urine, muscle and/or blood.

Step (c) may be performed using a DNA ligase, optionally a Splint R ligase. Step (d) may be performed by magnetic bead-based purification and/or exonuclease digestion of non-circularised padlock probes. The amplification in step (e) may be carried out by rolling circle amplification (RCA), optionally hyperbranched RCA (HRCA). Step (f) may be performed by detecting an increase in fluorescence, optionally the increase in fluorescence is detected in real-time. The method may further comprise a step (g) of quantifying the number of RNAs, optionally wherein the number of RNAs is quantified relative to a reference sample.

In another aspect, the present invention provides a method of detecting, diagnosing and/or assessing the clinical severity of a RNA-associated disease by determining the RNA mutation load.

The method may comprise:
(a) determining the concentration of wild-type RNAs (c_{wt});
(b) determining the concentration of mutant or aberrantly-modified RNAs (cₘᵤₜ); and
(c) calculating the percentage RNA mutation load or aberrant-modification load, cₘᵤₜ/(c_{wt}+cₘᵤₜ).

Suitably, the RNA-associated disease is a tRNA-associated disease and the RNAs are tRNAs, optionally wherein the RNA-associated disease is a mt-tRNA-associated disease and the RNAs are mt-tRNAs. Exemplary mt-tRNA-associated diseases are mitochondrial encephalopathy, lactic acidosis, and stroke-like episodes (MELAS) syndrome or myoclonic epilepsy with ragged red fibers syndrome (MERRF) syndrome.

In some embodiments, the concentration of mutant tRNAs is determined, and wherein, optionally: (i) the tRNAs are leucine(UUR) mt-tRNAs and the mutation is m.3243A>G; or (ii) the tRNAs are lysine mt-tRNAs and the mutation is m.8344A>G.

In some embodiments, the concentration of aberrantly-modified tRNAs is determined, and wherein, optionally: (i) the tRNAs are leucine(UUR) mt-tRNAs and the modified nucleotide is τm⁵U or (ii) the tRNAs are lysine mt-tRNAs and the modified nucleotide is τm⁵s²U.

Optionally, the method further comprises:
(d) determining the concentration of a reference RNA (c_{ref}); and
(e) calculating the relative quantity of wild-type RNA molecules, c_{wt}/c_{ref} and/or calculating the relative quantity of mutant or aberrantly-modified RNA molecules, cₘᵤₜ/c_{ref}.

Suitably, the reference RNA is a nuclear tRNA, optionally a nuclear methionine tRNA, or a mt-tRNA, optionally a methionine mt-tRNA.

One or more of c_{wt}, cₘᵤₜ, and c_{ref} may be determined by a method of the invention.

Optionally, the method further comprises:
(f) determining the concentration of wild-type RNA genes (c′_{wt});
(g) determining the concentration of mutant RNA genes (c′ₘᵤₜ); and
(h) calculating the percentage DNA mutation load, c′ₘᵤₜ/(c′_{wt}+c′ₘᵤₜ).

In another aspect, the present invention provides for use of a padlock probe in detecting and/or quantifying a mutant RNA. In a related aspect, the present invention provides for use of a padlock probe in detecting and/or quantifying an aberrantly modified RNA. In a related aspect, the present invention provides for use of a padlock probe in detecting and/or quantifying amino acid charging of a tRNA.

In another aspect, the present invention provides for use of a padlock probe in detecting, diagnosing and/or assessing the clinical severity of a RNA-associated disease.

Suitably, the RNA-associated disease is a tRNA-associated disease and the RNAs are tRNAs, optionally wherein the RNA-associated disease is a mt-tRNA-associated disease and the RNAs are mt-tRNAs. Exemplary mt-tRNA-associated diseases are mitochondrial encephalopathy, lactic acidosis, and stroke-like episodes (MELAS) syndrome or myoclonic epilepsy with ragged red fibers syndrome (MERRF) syndrome.

In another aspect, the present invention provides for use of a padlock probe for detecting and/or quantifying tRNA amino-acid charging.

### DESCRIPTION OF DRAWINGS

FIG. 1—Schematic illustration of mutation-dependent hybridization and ligation of a circularisable DNA probe

FIG. 2—Specific detection of three RNA samples.

The signals are measured during enzymatic amplification. Signal intensity increases with time. RNA concentration is smallest for the measurement shown as (*), increased by a factor of 10 for the measurement shown as (E) and by a further factor of 10 for the measurement shown as (o). The fluorescence signals are small at small time, then increase and finally saturate. The increase occurs earlier for higher RNA concentration. The time at which the relative signal passes a predetermined threshold is used to quantify the RNA concentration.

FIG. 3—Reference curves obtained with a series of standards

For (A) the wt DNA case, (B) the mut DNA case, (C) the wt RNA case, (D) the mut RNA case.

(A, B) The vertical axis (linear scale) shows the characteristic time cₜ in minutes, while the horizontal axis (logarithmic scale) shows the amount (copy number) of the synthetic wt or mut DNA introduced before the ligation. Three technical replica were done for each amount.

(C, D) The vertical axis shows the characteristic time cₜ in minutes, while the horizontal axis shows the amount (copy number) of the synthetic wt or mut RNA introduced before the ligation.

FIG. 4—Denaturing polyacrylamide gel (20%) of in vitro transcribed Leu UUR mt-tRNA.

Lane 2: WT Leu UUR mt-tRNA, Lane 3: Mut Leu UUR mt-tRNA. Lane 1 and 4: 50 bp DNA ladder (New England Biolabs).

### DETAILED DESCRIPTION

Various preferred features and embodiments of the present invention will now be described by way of non-limiting examples.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

Numeric ranges are inclusive of the numbers defining the range. Unless otherwise indicated, any nucleic acid sequences are written left to right in 5′ to 3′ orientation and amino acid sequences are written left to right in amino to carboxy orientation, respectively.

This disclosure is not limited by the exemplary methods and materials disclosed herein, and any methods and materials similar or equivalent to those described herein can be used in the practice or testing of embodiments of this disclosure.

The publications discussed herein are provided solely for their disclosure prior to the filing date of the present application. Nothing herein is to be construed as an admission that such publications constitute prior art to the claims appended hereto.

Ribonucleic Acid (RNA)

The present invention relates to methods of detecting and/or quantifying RNA.

Ribonucleic acid (RNA) is assembled as a chain of nucleotides. The RNA may be any type of RNA. Suitably, the RNA is transfer RNA (tRNA), ribosomal RNA (rRNA), messenger RNA (RNA), and/or regulatory RNA.

Transfer RNA (tRNA)

In preferred embodiments, the present invention relates to methods of detecting and/or quantifying a transfer RNA (tRNA).

A transfer RNA (abbreviated tRNA) is an adaptor molecule composed of RNA, typically 76 to 90 nucleotides in length, that delivers amino acids to the ribosome to translate the genetic information in an mRNA template-directed manner into a corresponding polypeptide chain (S. Kirchner, Z. Ignatova, Nat. Rev. Genet. 16, 98-112 (2015)).

tRNA molecules are synthesized as precursors that are then processed by a sequence of maturation events. These maturation events include removal of the 5′ leader, trimming of the 3′ trailer, splicing of introns, addition of the 3′-terminal CCA residues by a CCA-adding enzyme and covalent modification of multiple nucleoside residues (S. Kirchner, Z. Ignatova, Nat. Rev. Genet. 16, 98-112 (2015)).

Once charged with its cognate amino acid and in a complex with the elongation factor, each tRNA reaches the A-site of the ribosome and base-pairs its anticodon with the corresponding mRNA codon. Thus, to fit the same ribosomal site, the architecture of all tRNAs conform to a narrow set of structural parameters constrained by common identity rules and structural features. tRNAs are short, strongly structured and exhibit some chemical modifications, making detection challenging.

The tRNA may be any tRNA. Suitably, the tRNA may be a naturally-occurring tRNA. Suitably, the tRNA may be a human tRNA.

A method to specifically quantify tRNAs will have applications in research and development in biology, biotechnology and medicine, and in molecular diagnostics.

tRNAs vary significantly in their concentrations and codons pairing to low-abundance tRNAs are read slower than codons pairing to major tRNAs. The ribosomal speed is precisely regulated along the mRNA through selection of slow-translating codon clusters at specific positions which locally delay the synthesis rate and provide a time window for proper co-translational folding of the peptide chain. Consequently, silent mutations through an exchange of a slow codon for a fast and vice versa alter the local speed of translation and thus can also introduce aberrancies in protein folding.

Multicellular organisms carry over 500 different tRNA genes which differ in the codon they read (isoacceptors) or in the sequence of their body (isodecoders). Few examples suggest that tRNAs vary among tissues in the human organism, which may explain why some organs (including muscles, neuronal system) seem to be affected more than others. Furthermore, among all RNA species, tRNAs undergo by far the most and chemically diverse post-transcriptional modifications which control tRNA stability and modulate translation fidelity. Hypomodifications or a lack of modifications are also linked to complex human pathologies, including diabetes type-2 and various forms of mitochondrial encephalomyopathies. Despite its importance for deep understanding of the molecular pathology of many diseases, information on the variations in tRNA expression, abundance and modification pattern among different tissues (or organisms) is largely missing.

All tRNAs fit the same ribosomal site and their architecture conform to a narrow set of structural parameters constrained by common identity rules, i.e. narrow length range (73-90 nt) with large conservation. This is an inherent obstacle for tRNA identification. High-throughput RNA sequencing can be only used in part, because post-transcriptional modifications interfere with the PCR-based cDNA synthesis and change nucleotide identities. tRNA-based microarray technology is an alternative methodology, but is limited to only relative concentrations and can only distinguish tRNAs with more than 8 nt difference. The majority of the tRNA species, however, have less than 8 nt variations in the whole sequence. Currently, a technique for tRNA identification and quantification does not exist. With knowledge on the composition and concentration of the tRNAome of each species, cell or tissue, we may move to a more accurate understanding of the molecular basis of silent mutations-based pathologies and tissue-specific aspects that modulate disease severity and progression.

In addition, tRNA sets largely differ among organisms, an obstacle for overexpression of proteins with therapeutic or industrial applications. Thus, knowledge of the tRNAome of a parental strain, donating the protein to be overexpressed, and of the expression host will allow synchronization of translation patterns between organisms. Specifically, using silent mutations the original translation pattern within the parental strain can be adapted to the tRNAome of the expression host and consequently will increase yield of a soluble and active heterologous protein.

Mitochondrial-Encoded tRNAs (Mt-tRNAs)

In preferred embodiments of the present invention, the RNA is a mitochondrial-encoded tRNA (mt-tRNA).

The mt-tRNA may be any mt-tRNA. Suitably, the mt-tRNA may be a naturally-occurring and/or human mt-tRNA.

Mitochondria are organelles found in most eukaryotic cells. Each cell contains hundreds to thousands of mitochondria and each contains several copies of the mitochondrial DNA (mtDNA) genome. Human mtDNA is a closed circular double-stranded DNA with 16,569 base pairs encoding 37 genes: 13 for the essential subunits of respiratory complexes I, III, IV, and V; 22 for tRNAs (mt-tRNAs); and two for rRNAs (mt-rRNAs). Therefore, all the RNA components necessary for mitochondrial translation are supplied in mitochondria, whereas all protein components, including ribosomal proteins, translational factors, aminoacyl-tRNA synthetases, and various factors required for the biogenesis of tRNAs and rRNAs, are encoded in the nucleus and transported into the mitochondria subsequent to their synthesis in the cytoplasm (T. Suzuki et al, Annu. Rev. Genet. 45, 299-329 (2011)).

Mitochondrial-encoded tRNAs (mt-tRNAs) show broader structural heterogeneity than nuclear-encoded tRNAs (S. Kirchner, Z. Ignatova, Nat. Rev. Genet. 16, 98-112 (2015)). Further, for all the identified cases in which human disease are directly linked to mutations in tRNAs, the mutations occur in mt-tRNAs. A possible explanation is that a mutated mt-tRNA is unlikely to be compensated for by other tRNAs: each mitochondrial genome bears a single copy of only 22 mt-tRNAs, and importing a nuclear-encoded tRNA is rare (S. Kirchner, Z. Ignatova, Nat. Rev. Genet. 16, 98-112 (2015)).

Pathogenic point mutations have been described that affect every mt-tRNA and are linked to defects in oxidative energy metabolism. It is now emerging that these mutations are also linked to other complex traits, including neurodegenerative diseases, ageing and cancer (E. A. Schon et al, Nat. Rev. Genet. 13, 878-890 (2012)). Mutations are catalogued in variety of databases, such as MITOMAP and Online Mendelian Inheritance in Man (OMIM). MELAS and MERRF are two classic diseases associated with mitochondrial tRNA mutations in mt-tRNA Leu(UUR) and mt-tRNA Lys, respectively. Other mtDNA mutations associated with the MERRF and MELAS phenotypes are reported in Yarham, J. W., et al., 2010. Wiley Interdisciplinary Reviews: RNA, 1(2), pp. 304-324.

If all mitochondrial genomes in one cell carry a mutation, a condition known as homoplasmy, the effect of this mutation is enhanced. However, a pathogenic mutation in mt-tRNA may also affect a proportion of the mt-tRNA copies, a condition known as heteroplasmy. Manifestation of a clinical phenotype depends on the threshold of mutation-affected mitochondria; this threshold varies among tissues. (S. Kirchner, Z. Ignatova, Nat. Rev. Genet. 16, 98-112 (2015)).

Suitably, the mt-tRNA may be selected from one or more of: a leucine(UUR) mt-tRNA, a lysine mt-tRNA, a methionine mt-tRNA, a tryptophan mt-tRNA, an aspartate mt-tRNA, an isoleucine mt-tRNA, a glycine mt-tRNA, an arginine mt-tRNA, a histidine mt-tRNA, a serine(AGY) mt-tRNA, a leucine(CUN) mt-tRNA, a threonine mt-tRNA, a phenylalanine mt-tRNA, a valine mt-tRNA, a glutamine mt-tRNA, an alanine mt-tRNA, an asparagine mt-tRNA, a cysteine mt-tRNA, a tyrosine mt-tRNA, a serine(UCN) mt-tRNA, a glutamate mt-tRNA, and a proline mt-tRNA.

In some embodiments, the mt-tRNA is selected from one or more of: a leucine(UUR) mt-tRNA, a lysine mt-tRNA, a histidine mt-tRNA, a leucine(CUN) mt-tRNA, a phenylalanine mt-tRNA, a valine mt-tRNA, a glutamine mt-tRNA, a serine(UCN) mt-tRNA, or a proline mt-tRNA.

In some embodiments, the mt-tRNA is selected from a leucine(UUR) mt-tRNA or a lysine mt-tRNA.

In some embodiments, the mt-tRNA is a leucine(UUR) mt-tRNA.

Exemplary mt-tRNA consensus sequences are given in Table 1, below. These sequences do not show the 3′-terminal CCA residues. The mt-tRNA may comprise CCA residues at the 3′ terminal of the exemplary mt-tRNA sequences.

Suitably, the mt-tRNA comprises a nucleotide sequence selected from one or more of SEQ ID NOs: 1-22, or a variant with at least 90%, at least 95%, or at least 98% sequence identity to one or more of SEQ ID NOs: 1-22.

In some embodiments, the mt-tRNA comprises a nucleotide sequence selected from one or more of SEQ ID NOs: 1, 2, 9, 11, 13, 14, 15, 20, 22, or a variant with at least 90%, at least 95%, or at least 98% sequence identity to one or more of SEQ ID NOs: 1, 2, 9, 11, 13, 14, 15, 20, 22.

In some embodiments, the mt-tRNA comprises a nucleotide sequence selected from SEQ ID NO: 1 or SEQ ID NO: 2, or a variant with at least 90%, at least 95%, or at least 98% sequence identity to SEQ ID NO: 1 or SEQ ID NO: 2.

In some embodiments, the mt-tRNA comprises the nucleotide sequence of SEQ ID NO: 1, or a variant with at least 90%, at least 95%, or at least 98% sequence identity to SEQ ID NO: 1.

TABLE 1
Exemplary mt-tRNA consensus sequences

| | RNA | |
|---|---|---|
| | central | Exemplary |
| mt-tRNA | ref no. | consensus sequence |
| leucine | URS000061A10B | GUUAAGAUGGCAGAGCCCGGUAAUCGC |
| (UURname) | | AUAAAACUUAAAACUUUACAGUCAGAGG |
| mt-tRNA | | UUCAAUUCCUCUUCUUAACA |
| (SEQ ID | | |
| NO: 1) | | |
| lysine | URS000007A90A | CACUGUAAAGCUAACUUAGCAUUAACCU |
| mt-tRNA | | UUUAAGUUAAAGAUUAAGAGAACCAACA |
| (SEQ ID | | CCUCUUUACAGUGA |
| NO: 2) | | |
| methio- | URS000006E464 | AGUAAGGUCAGCUAAAUAAGCUAUCGG |
| nine | | GCCCAUACCCCGAAAAUGUUGGUUAUA |
| mt-tRNA | | CCCUUCCCGUACUA |
| (SEQ ID | | |
| NO: 3) | | |
| trypto- | URS000012396D | AGAAAUUUAGGUUAAAUACAGACCAAGA |
| phan | | GCCUUCAAAGCCCUCAGUAAGUUGCAA |
| mt-tRNA | | UACUUAAUUUCUG |
| (SEQ ID | | |
| NO: 4) | | |
| aspartate | URS00001FC4D3 | AAGGUAUUAGAAAAACCAUUUCAUAACU |
| mt-tRNA | | UUGUCAAAGUUAAAUUAUAGGCUAAAUC |
| (SEQ ID | | CUAUAUAUCUUA |
| NO: 5) | | |
| isoleu- | URS000025082B | AGAAAUAUGUCUGAUAAAAGAGUUACUU |
| cine | | UGAUAGAGUAAAUAAUAGGAGCUUAAAC |
| mt-tRNA | | CCCCUUAUUUCUA |
| (SEQ ID | | |
| NO: 6) | | |
| glycine | URS00000C6674 | ACUCUUUUAGUAUAAAUAGUACCGUUAA |
| mt-tRNA | | CUUCCAAUUAACUAGUUUUGACAACAUU |
| (SEQ ID | | CAAAAAAGAGUA |
| NO: 7) | | |
| arginine | URS00005983A6 | UGGUAUAUAGUUUAAACAAAACGAAUGA |
| mt-tRNA | | UUUCGACUCAUUAAAUUAUGAUAAUCAU |
| (SEQ ID | | AUUUACCAA |
| NO: 8) | | |
| histidine | URS00001FBD75 | GUAAAUAUAGUUUAACCAAAACAUCAGA |
| mt-tRNA | | UUGUGAAUCUGACAACAGAGGCUUACG |
| (SEQ ID | | ACCCCUUAUUUACC |
| NO: 9) | | |
| serine | URS00002C130C | GAGAAAGCUCACAAGAACUGCUAACUCA |
| (AGY) | | UGCCCCCAUGUCUAACAACAUGGCUUU |
| mt-tRNA | | CUCA |
| (SEQ ID | | |
| NO: 10) | | |
| leucine | URS000056BD99 | ACUUUUAAAGGAUAACAGCUAUCCAUUG |
| (CUN) | | GUCUUAGGCCCCAAAAAUUUUGGUGCA |
| mt-tRNA | | ACUCCAAAUAAAAGUA |
| (SEQ ID | | |
| NO: 11) | | |
| threonine | URS00000FCDE9 | GUCCUUGUAGUAUAAACUAAUACACCAG |
| mt-tRNA | | UCUUGUAAACCGGAGAUGAAAACCUUU |
| (SEQ ID | | UUCCAAGGACA |
| NO: 12) | | |
| phenyla- | URS00003E8921 | GUUUAUGUAGCUUACCUCCUCAAAGCA |
| lanine | | AUACACUGAAAAUGUUUAGACGGGCUC |
| mt-tRNA | | ACAUCACCCCAUAAACA |
| (SEQ ID | | |
| NO: 13) | | |
| valine | URS00002D2D8F | CAGAGUGUAGCUUAACACAAAGCACCCA |
| mt-tRNA | | ACUUACACUUAGGAGAUUUCAACUUAAC |
| (SEQ ID | | UUGACCGCUCUGA |
| NO: 14) | | |
| glutamine | URS000019B78E | UAGGAUGGGGUGUGAUAGGUGGCACG |
| mt-tRNA | | GAGAAUUUUGGAUUCUCAGGGAUGGGU |
| (SEQ ID | | UCGAUUCUCAUAGUCCUAG |
| NO: 15) | | |
| alanine | URS000036D40A | AAGGGCUUAGCUUAAUUAAAGUGGCUG |
| mt-tRNA | | AUUUGCGUUCAGUUGAUGCAGAGUGGG |
| (SEQ ID | | GUUUUGCAGUCCUUA |
| NO: 16) | | |
| aspara- | URS00004206E2 | UAGAUUGAAGCCAGUUGAUUAGGGUGC |
| gine | | UUAGCUGUUAACUAAGUGUUUGUGGGU |
| mt-tRNA | | UUAAGUCCCAUUGGUCUAG |
| (SEQ ID | | |
| NO: 17) | | |
| cysteine | URS000018E119 | AGCUCCGAGGUGAUUUUCAUAUUGAAU |
| mt-tRNA | | UGCAAAUUCGAAGAAGCAGCUUCAAACC |
| (SEQ ID | | UGCCGGGGCUU |
| NO: 18) | | |
| tyrosine | URS00000F3F5E | GGUAAAAUGGCUGAGUGAAGCAUUGGA |
| mt-tRNA | | CUGUAAAUCUAAAGACAGGGGUUAGGC |
| (SEQ ID | | CUCUUUUUACCA |
| NO: 19) | | |
| serine | URS000025B782 | GAAAAAGUCAUGGAGGCCAUGGGGUUG |
| (UCN) | | GCUUGAAACCAGCUUUGGGGGGUUCGA |
| mt-tRNA | | UUCCUUCCUUUUUUG |
| (SEQ ID | | |
| NO: 20) | | |
| glutamate | URS000034E9D0 | GUUCUUGUAGUUGAAAUACAACGAUGG |
| mt-tRNA | | UUUUUCAUAUCAUUGGUCGUGGUUGUA |
| (SEQ ID | | GUCCGUGCGAGAAUA |
| NO: 21) | | |
| proline | URS000002176F | CAGAGAAUAGUUUAAAUUAGAAUCUUAG |
| mt-tRNA | | CUUUGGGUGCUAAUGGUGGAGUUAAAG |
| (SEQ ID | | ACUUUUUCUCUGA |
| NO: 22) | | |

Suitably, a mt-tRNA Leu(UUR) consists of the nucleotide sequence of SEQ ID NO: 23, or a variant with at least 90%, at least 95%, or at least 98% sequence identity to SEQ ID NO: 23;

Exemplary consensus mt-tRNA Leu(UUR) sequence
with 3′-terminal CCA residues
(SEQ ID NO: 23)
GUUAAGAUGGCAGAGCCCGGUAAUCGCAUAAAACUUAAAACUUUACAGUC
AGAGGUUCAAUUCCUCUUCUUAACACCA

Suitably, a mt-tRNA Lys consists of the nucleotide sequence of SEQ ID NO: 24, or a variant with at least 90%, at least 95%, or at least 98% sequence identity SEQ ID NO: 24.

Exemplary consensus mt-tRNA Lys sequence
with 3′-terminal CCA residues
(SEQ ID NO: 24)
CACUGUAAAGCUAACUUAGCAUUAACCUUUUAAGUUAAAGAUUAAGAGAA
CCAACACCUCUUUACAGUGACCA

tRNA Amino Acid-Charging

Mature eukaryotic tRNAs are prepared to function as an adaptor in translation by the covalent attachment of an amino acid to the adenosine at the invariant 3′ CCA tail. This reaction is catalysed by 20 different aminoacyl tRNA synthetases (ARSs), each of which is specific for the 20 different amino acids (S. Kirchner, Z. Ignatova, Nat. Rev. Genet. 16, 98-112 (2015)). This process is known as known as Amino acid activation, aminoacylation or tRNA amino acid-charging.

Mutations in the tRNA itself (such as those discussed above) can disrupt the tRNA charging activity or lead to mischarging of the tRNA. Further, mutations in the ARS genes can result in reduced tRNA charging activity, complete loss of tRNA charging activity, and/or mischarging of tRNA.

In human cells, two distinct sets of ARSs are distinguished by their cytoplasmic or mitochondrial localization. Human cells contain 17 cytoplasmic ARS polypeptides, 18 mitochondrial ARSs, and 2 dual-localized ARSs present in both cytoplasm and mitochondria.

Mutations in cytoplasmic ARSs are associated with Charcot-Marie-Tooth (CMT) and related neuropathies. Mutations in mitochondrial ARSs are associated with a wider variety of syndromes and diseases (Yao, P. and Fox, P. L., 2013. EMBO molecular medicine, 5(3), pp. 332-343).

To detect and/or quantify tRNAs which are not amino acid-charged, an oligonucleotide can be ligated to the 3′ end of the tRNA. This ligation is possible only if the tRNA is not AA-charged, since otherwise the 3′ end of the tRNA is not free but bound to the amino acid. Subsequently, the presence of a nucleotide sequence located in the oligonucleotide can be detected and/or quantified.

The oligonucleotide is suitably a heteroduplex composed of an RNA oligonucleotide and a DNA oligonucleotide (i.e. an RNA/DNA heteroduplex) or a DNA oligonucleotide duplex. Suitably, the (hetero)duplex is formed from two complementary oligonucleotides and/or comprises a TGG-3′ overhang. Optionally, the oligonucleotide is an RNA/DNA heteroduplex comprising a TGG-3′ overhang of the DNA oligonucleotide. For example, the oligonucleotide may consist of: (i) a DNA oligonucleotide consisting of from 5′ to 3′: (N)ₓ-TGG, where x is 1 to 100, 5 to 50, or 10 to 30; and (ii) a RNA oligonucleotide comprising or consisting of the reverse complement of (N)ₓ.

Accordingly, in some embodiments the tRNA comprises a 3′ ligated oligonucleotide. Suitably, the tRNA comprises a sequence comprising or consisting of from 5′ to 3′: SEQ-CCA-(N)ₓ, where x is 1 to 100, 5 to 50, or 10 to 30. SEQ may be any tRNA sequence, for example a nucleotide sequence selected from one or more of SEQ ID NOs: 1-22, or a variant with at least 90%, at least 95%, or at least 98% sequence identity to one or more of SEQ ID NOs: 1-22. The presence of the sequence (N)ₓ (i.e. the 3′ ligated oligonucleotide) can be detected and/or quantified.

Other RNAs

In some embodiments, the present invention relates to methods of detecting and/or quantifying a ribosomal RNA (rRNA).

Ribosomal RNA (rRNA) is a type of non-coding RNA which is the primary component of ribosomes. The rRNA may be any rRNA. Suitably, the rRNA may be a naturally-occurring rRNA. Suitably, the rRNA may be a human rRNA. Suitably, the rRNA may be a mitochondrially-encoded rRNA.

In some embodiments, the present invention relates to methods of detecting and/or quantifying a messenger RNA (mRNA).

Messenger RNA (mRNA) is a single-stranded RNA molecule that corresponds to the genetic sequence of a gene and is read by the ribosome in the process of producing a protein. The mRNA may be any mRNA. Suitably, the mRNA may be a naturally-occurring mRNA.

Suitably, the mRNA may be a human mRNA. Suitably, the mRNA may be a mitochondrially-encoded mRNA.

In some embodiments, the present invention relates to methods of detecting and/or quantifying a regulatory RNA.

Regulatory RNAs are non-coding RNA molecules that play a role in cellular processes such as activation or inhibition processes. Regulatory RNAs include microRNA (miRNAs) and their precursors, small interfering RNAs (siRNAs), short hairpin RNAs (shRNAs), and antisense RNAs (asRNAs). Other regulatory RNAs are described in Morris, K. V. and Mattick, J. S., 2014. Nature Reviews Genetics, 15(6), pp. 423-437. The regulatory RNA may be any regulatory RNA. Suitably, the regulatory RNA may be a naturally-occurring regulatory RNA. Suitably, the regulatory RNA may be a human regulatory RNA.

RNA Mutations

The RNA may comprise one or more mutations, i.e. the RNA may be a mutant RNA. A “mutation” is an alteration in the nucleotide sequence compared to the consensus nucleotide sequence (e.g. SEQ ID NOs: 1-24). Suitably, the mutation may be a point mutation, an insertion, a deletion, or duplication.

The RNA may comprise one or more pathogenic mutations, i.e. the RNA may be a pathogenic mutant RNA. A “pathogenic mutation” is a mutation that increases an individual's susceptibility or predisposition to a certain disease or disorder.

Suitably, the mutation is a point mutation, i.e. the RNA may comprise one or more point mutations, optionally one or more pathogenic point mutations. A “point mutation” is a mutation that only affects a single nucleotide of the nucleotide sequence.

The RNA may comprise one or more heteroplasmic or homoplasmic mutations, particularly when the RNA is mitochondrially-encoded (e.g. an mt-tRNA). A cell can have some mitochondria that have a mutation in the mtDNA and some that do not. In addition, a mitochondria can have some copies of mtDNA that have a mutation and some copies that do not. This is termed heteroplasmy. A “heteroplasmic mutation” is a mutation which can be present in a proportion of the mtDNA, and thus in a proportion of the mt-RNA. Most common mt-tRNA mutations are heteroplasmic. Homoplasmy refers to a cell and/or mitochondria that has a uniform collection of mtDNA. A “homoplasmic mutation” is a mutation which can be present in all the mtDNA, and thus in all the mt-RNA. Suitably, the RNA (e.g. mt-tRNA) comprises a heteroplasmic mutation.

mt-RNA mutations may be referred to based on the corresponding position in the mt-DNA rather than the position in the mt-RNA. For example, the A3243G mt-DNA mutation corresponds to an A14G point mutation in leucine(UUR) mt-tRNA and the A8344G mt-DNA mutation corresponds to an A55G point mutation in lysine mt-tRNA. A conversion of nucleotide numbering in the human mitochondrial genome to conventional tRNA numbering is provided by Mamit-tRNA (Joern Putz, Bruno Dupuis, Marie Sissler and Catherine Florentz, (2007), RNA, 13, pp 1184-90).

Exemplary mt-tRNA mutations, including mt-tRNA pathogenic mutations are provided by MITOMAP.

Exemplary mt-tRNA pathogenic mutations for each mt-tRNA are given in Table 2, below. An mt-tRNA may comprise one or more pathogenic mutation recited in Table 2. Suitably, an mt-tRNA may comprise a nucleotide sequence selected from any of SEQ ID NOs: 1-24 and a corresponding pathogenic mutation recited in Table 2.

TABLE 2
Exemplary mt-tRNA pathogenic mutations

| | mt-DNA | |
|---|---|---|
| mt-tRNA name | gene | Pathogenic mutations |
| leucine(UUR) mt-tRNA | MT-TL1 | A3236G, G3242A, A3243G, A3243T, G3244A, |
| (SEQ ID NO: 1) | | G3249A, T3250C, A3251G, A3252G, A3252T, |
| | | T3253C, C3254A, C3254G, C3254T, G3255A, |
| | | C3256T, T3258C, A3260G, T3264C, T3271del, |
| | | T3271C, T3273C, A3274G, C3275A, C3275T, |
| | | G3277A, T3278C, A3280G, G3283A, C3287A, |
| | | A3288G, T3290C, T3291C, A3302G, C3303T |
| lysine mt-tRNA | MT-TK | A8296G, G8299A, A8302T, G8304A, C8305T, |
| (SEQ ID NO: 2) | | T8306C, T8311C, G8313A, T8316C, A8319G, |
| | | A8326G, G8328A, A8332G, T8337C, G8340A, |
| | | G8342A, A8343G, A8344G, A8347G, A8348G, |
| | | T8355C, T8356C, T8357C, G8361A, T8362G, |
| | | G8363A |
| methionine mt-tRNA | MT-TM | G4403A, T4409C, C4410A, G4412A, A4415G, |
| (SEQ ID NO: 3) | | A4435G, C4437T, G4440A, G4450A, T4454C, |
| | | C4456T, C4467A |
| tryptophan mt-tRNA | MT-TW | A5512G, G5513A, A5514G, G5521A, G5522A, |
| (SEQ ID NO: 4) | | T5523G, G5532A, A5537insT, G5538A, G5540A, |
| | | C5541T, T5543C, C5545T, G5549A, G5556A, |
| | | G5556C, A5559G, T5567C, A5568G |
| aspartate mt-tRNA | MT-TD | G7520A, A7526G, C7539T, A7543G, A7551G, |
| (SEQ ID NO: 5) | | G7554A, G7566A |
| isoleucine mt-tRNA | MT-TI | A4263G, A4267G, A4269G, T4274C, T4277C, |
| (SEQ ID NO: 6) | | A4279G, A4281G, G4282A, G4284A, T4285C, |
| | | T4289C, T4290C, T4291C, A4295G, G4296A, |
| | | G4298A, A4300G, A4302G, G4308A, G4309A, |
| | | T4314C, A4316G, A4317del, A4317G, C4320T, |
| | | C4322del, C4322CC |
| glycine mt-tRNA | MT-TG | T9997A, T9997C, T10003C, A10006G, T10010C, |
| (SEQ ID NO: 7) | | G10014A, A10044G |
| arginine mt-tRNA | MT-TR | G10406A, T10408C, A10411T, T10415C, G10437A, |
| (SEQ ID NO: 8) | | A10438G, A10450G, T10454C, T10460C |
| histidine mt-tRNA | MT-TH | A12146G, G12147A, T12148C, A12182G, G12183A, |
| (SEQ ID NO: 9) | | C12187A, G12192A, T12201C, C12206T |
| serine(AGY) mt-tRNA | MT-TS2 | G12207A, C12224T, G12236A, C12246A, C12258A, |
| (SEQ ID NO: 10) | | C12258T, T12261C, C12262A, C12264T |
| leucine(CUN) mt-tRNA | MT-TL2 | G12276A, A12280G, G12283A, G12293A, G12294A, |
| (SEQ ID NO: 11) | | T12297C, A12299C, G12300A, A12308G, T12311C, |
| | | T12313C, G12315A, G12316A, T12317C, A12320G, |
| | | T12335C |
| threonine mt-tRNA | MT-TT | G15894A, T15908C, A15909G, G15915A, A15923G, |
| (SEQ ID NO: 12) | | A15924G, G15927A, G15928A, G15933A, T15942C, |
| | | T15944del, G15950A, A15951G |
| phenylalanine mt-tRNA | MT-TF | T578C, T582C, G583A, G586A, T593C, C602T, |
| (SEQ ID NO: 13) | | A606G, A608G, G611A, T616C, T616G, G617A, |
| | | T618C, T618G, G622A, G625A, C628T, A636G, |
| | | A641T, T642C |
| valine mt-tRNA | MT-TV | G1606A, T1607C, A1616G, C1624T, A1630G, |
| (SEQ ID NO: 14) | | G1642A, A1643G, G1644A, G1644T, T1659C |
| glutamine mt-tRNA | MT-TQ | G4332A, T4336C, A4343G, C4345T, T4353C, |
| (SEQ ID NO: 15) | | T4363C, A4369AA, C4372T, T4373C, A4381G, |
| | | T4386C, C4387A, A4388G, C4392T, A4395G |
| alanine mt-tRNA | MT-TA | T5587C, G5591A, A5592G, G5610A, T5613C, |
| (SEQ ID NO: 16) | | T5628C, G5631A, T5636C, G5650A, C5652G, |
| | | T5655C |
| asparagine mt-tRNA | MT-TN | T5658C, G5667A, A5690G, T5692C, T5693C, |
| (SEQ ID NO: 17) | | G5698A, G5703A, T5709C, T5728C |
| cysteine mt-tRNA | MT-TC | G5780A, G5783A, T5802C, T5814C, A5816G, |
| (SEQ ID NO: 18) | | G5821A |
| tyrosine mt-tRNA | MT-TY | A5843G, T5874G, A5889G |
| (SEQ ID NO: 19) | | |
| serine(UCN) mt-tRNA | MT-TS1 | A7451T, G7453A, A7456G, G7458A, C7462T, |
| (SEQ ID NO: 20) | | C7471CC, A7472C, A7472CA, T7480G, G7486A, |
| | | C7492T, G7497A, T7501A, T7505C, G7506A, |
| | | T7510C, T7511C, T7512C |
| glutamate mt-tRNA | MT-TE | T14674C, T14674G, C14680A, G14685A, A14687G, |
| (SEQ ID NO: 21) | | A14692G, A14693G, A14696G, T14709C, G14710A, |
| | | G14721A, T14723C, G14724A, T14728C, G14739A |
| proline mt-tRNA | MT-TP | A15965G, G15967A, C15975T, C15990T, G15995A, |
| (SEQ ID NO: 22) | | A15998T, T16002C, T16015C, |
| | | T16018TTCTCTGTTCTTTCAT, 16021_16022delCT, |
| | | G16023A, T16032TTCTCTGTTCTTTCAT, |
| | | G16033TCTCTGTTCTTTCATG |

Exemplary confirmed mt-tRNA pathogenic mutations for each mt-tRNA are given in Table 3 below. Confirmed pathogenic mutations have confirming reports which address criteria including: (1) independent reports of two or more unrelated families with evidence of similar disease; (2) evolutionary conservation of the nucleotide (for RNA variants) or amino acid (for coding variants); (3) presence of heteroplasmy; (4) correlation of variant with phenotype/segregation of the mutation with the disease within a family; (5) biochemical defects in complexes I, III, or IV in affected or multiple tissues; (6) functional studies showing differential defects segregating with the mutation (cybrid or single fiber studies); (7) histochemical evidence of a mitochondrial disorder; and (8) for fatal or severe phenotypes, the absence or extremely rare occurrence of the variant in large mtDNA sequence databases. An mt-tRNA may comprise one or more pathogenic mutation recited in Table 3. Suitably, an mt-tRNA may comprise a nucleotide sequence selected from any of SEQ ID NOs: 1-24 and a corresponding pathogenic mutation recited in Table 3.

TABLE 3
exemplary confirmed mt-tRNA pathogenic mutations

| | mt-DNA | |
|---|---|---|
| mt-tRNA name | gene | Confirmed pathogenic mutations |
| leucine(UUR) mt-tRNA | MT-TL1 | A3243G, A3243T, C3256T, T3258C, |
| | | A3260G, T3271del, T3271C, A3280G, |
| | | T3291C, A3302G |
| lysine mt-tRNA | MT-TK | T8306C, G8313A, G8340A, A8344G, |
| | | T8356C, G8363A |
| methionine mt-tRNA | MT-TM | G4450A |
| tryptophan mt-tRNA | MT-TW | G5521A, A5537insT |
| isoleucine mt-tRNA | MT-TI | G4298A, A4300G, G4308A |
| glycine mt-tRNA | MT-TG | T10010C |
| histidine mt-tRNA | MT-TH | G12147A |
| serine(AGY) mt-tRNA | MT-TS2 | C12258A |
| leucine(CUN) mt-tRNA | MT-TL2 | G12276A, G12294A, G12315A, |
| | | G12316A |
| phenylalanine mt-tRNA | MT-TF | G583A, T616C |
| valine mt-tRNA | MT-TV | G1606A, A1630G, G1644A |
| glutamine mt-tRNA | MT-TQ | G4332A |
| alanine mt-tRNA | MT-TA | G5650A |
| asparagine mt-tRNA | MT-TN | A5690G, G5703A, T5728C |
| serine(UCN) mt-tRNA | MT-TS1 | C7471CC, G7497A, T7510C, T7511C |
| glutamate mt-tRNA | MT-TE | T14674C, T14709C, G14710A |

Exemplary mt-tRNA pathogenic mutations for different diseases are given in Table 4 below. An mt-tRNA may comprise one or more pathogenic mutation recited in Table 4. Suitably, an mt-tRNA may comprise a nucleotide sequence selected from any of SEQ ID NOs: 1-24 and a corresponding pathogenic mutation recited in Table 4.

TABLE 4
exemplary mt-tRNA pathogenic mutations and associated disease

| Disease | mt-tRNA | Mutation |
|---|---|---|
| Maternal Myopathy and Cardiomyopathy (MMC) | mt-tRNA Leu (UUR) | A3260G |
| Maternal Myopathy and Cardiomyopathy (MMC) | mt-tRNA Leu (UUR) | C3303T |
| MICM: Maternally Inherited Cardiomyopathy (MICM) | mt-tRNA Ile | A4300G |
| Sensorineural Hearing Loss (SNHL) | mt-tRNA Ser (UCN) | T7511C |
| Diabetes Mellitus & Deafness/SNHL/Focal | mt-tRNA Leu (UUR) | A3243G |
| Segmental Glomerulosclerosis/Cardiac and multi- | | |
| organ dysfunction | | |
| Mitochondrial Encephalomyopathy, Lactic Acidosis, | mt-tRNA Phe | G583A |
| and Stroke-like episodes (MELAS), Mitochondrial | | |
| Myopathy (MM) and Exercise Intolerance (EXIT) | | |
| Ataxia, Myoclonus and Deafness (AMDF) | mt-tRNA Val | G1606A |
| MELAS and Leigh Syndrome (LS) | mt-tRNA Leu (UUR) | A3243G |
| MELAS | mt-tRNA Leu (UUR) | C3256T |
| MELAS | mt-tRNA Leu (UUR) | T3271C |
| MELAS/Myopathy/Deafness and Cognitive | mt-tRNA Leu (UUR) | T3291C |
| Impairment | | |
| MELAS/encephalopathy | mt-tRNA Gln | G4332A |
| Maternally Inherited Leigh Syndrome (MILS) | mt-tRNA Trp | A5537insT |
| Progressive Encephalopathy (PEM)/AMDF/Motor | mt-tRNA Ser (UCN) | C7471CC |
| neuron disease-like | | |
| Myoclonic Epilepsy and Ragged Red Muscle Fibers | mt-tRNA Lys | A8344G |
| (MERRF) | | |
| MERRF | mt-tRNA Lys | T8356C |
| MERRF/MICM/deafness/autism/LS/Ataxia and | mt-tRNA Lys | G8363A |
| Lipomas | | |
| Progressive Encephalopathy (PEM) | mt-tRNA Gly | T10010C |
| MERRF-MELAS/cerebral edema | mt-tRNA His | G12147A |
| Mitochondrial Myopathy (MM)/CPEO (Chronic | mt-tRNA Leu (UUR) | A3243G |
| Progressive External Ophthalmoplegia) | | |
| Mitochondrial Myopathy (MM) | mt-tRNA Leu (UUR) | A3302G |
| Chronic Progressive External Ophthalmoplegia | mt-tRNA Ile | G4298A |
| (CPEO)/MS | | |
| Chronic Progressive External Ophthalmoplegia | mt-tRNA Ile | G4308A |
| (CPEO) | | |
| Myopathy | mt-tRNA Ala | G5650A |
| Chronic Progressive External Ophthalmoplegia | mt-tRNA Asn | G5703A |
| (CPEO)/ Mitochondrial Myopathy (MM) | | |
| Mitochondrial Myopathy (MM)/Exercise Intolerance | mt-tRNA Ser (UCN) | G7497A |
| (EXIT) | | |
| CPEO/Kearns Sayre Syndrome (KSS) | mt-tRNA Leu (CUN) | G12315A |
| Reversible COX deficiency myopathy | mt-tRNA Glu | T14674C |
| Mitochondrial Myopathy, Diabetes Mellitus and | mt-tRNA Glu | T14709C |
| Encephalomyopathy | | |

An mt-tRNA may be selected from the following mutant mt-tRNAs, which are associated with MELAS and MERRF:
(i) a mt-tRNA Leu (UUR) with one or more mutation selected from A3243G, C3256T, T3271C, and T3291C;
(ii) a mt-tRNA Phe with mutation G583A;
(iii) a mt-tRNA Gln with mutation G4332A;
(iv) a mt-tRNA His with mutation G12147A; and
(v) a mt-tRNA Lys with one or more mutation selected from A8344G, T8356C, G8363A.

An mt-tRNA may be selected from the following mutant mt-tRNAs, which are associated with MELAS:
(i) a mt-tRNA Leu (UUR) with one or more mutation selected from A3243G, C3256T, T3271C, and T3291C;
(ii) a mt-tRNA Phe with mutation G583A;
(iii) a mt-tRNA Gln with mutation G4332A; and
(iv) a mt-tRNA His with mutation G12147A.

In some embodiments, the mt-tRNA is mt-tRNA Leu (UUR) with mutation A3243G or T3271C, or mt-tRNA Lys with mutation A8344G. In some embodiments, the mt-tRNA is mt-tRNA Leu (UUR) with mutation A3243G.

Suitably, an mt-tRNA Leu (UUR) with mutation A3243G may consist of SEQ ID NO: 25, or a variant with at least 90%, at least 95%, or at least 98% sequence identity to SEQ ID NO: 25.

Exemplary mt-tRNA Leu (UUR) with mutation A3243G
(SEQ ID NO: 25)
GUUAAGAUGGCAGGGCCCGGUAAUCGCAUAAAACUUAAAACUUUACA
GUCAGAGGUUCAAUUCCUCUUCUUAACACCA

Suitably, an mt-tRNA Leu (UUR) with mutation T3271C may consist of SEQ ID NO: 26, or a variant with at least 90%, at least 95%, or at least 98% sequence identity to SEQ ID NO: 26.

Exemplary mt-tRNA Leu (UUR) with mutation T3271C
(SEQ ID NO: 26)
GUUAAGAUGGCAGAGCCCGGUAAUCGCAUAAAACUUAAAACCUUACA
GUCAGAGGUUCAAUUCCUCUUCUUAACA

Suitably, an mt-tRNA Lys with mutation A8344G may consist of SEQ ID NO: 27, or a variant with at least 90%, at least 95%, or at least 98% sequence identity to SEQ ID NO: 27.

Exemplary mt-tRNA Lys with mutation A8344G
(SEQ ID NO: 27)
CACUGUAAAGCUAACUUAGCAUUAACCUUUUAAGUUAAAGAUUAAGA
GAGCCAACACCUCUUUACAGUGACCA

In SEQ ID NOs: 25-27 the position of the mutation is shown in bold and underlined.

RNA Post-Transcriptional Modifications

The RNA may comprise one or more post-transcriptional modifications. A “post-transcriptional modification” is any chemical modification of the nucleosides in the RNA following transcription.

RNA can be post-transcriptionally modified with over 100 chemical moieties. For example, on average a tRNA can carry 14 modifications that contribute to its function. Modifications can directly influence RNA structure, by promoting or disrupting certain intramolecular interactions; they can make the RNA molecule more rigid or more flexible. They can also influence RNA interactions with other molecules, in particular proteins. Post-transcriptional RNA modifications are described in the MODOMICS database. (Boccaletto, P., et al., 2018. Nucleic acids research, 46(D1), pp. D303-D307.).

For example, to be fully active, tRNAs need to be heavily modified post-transcriptionally. Growing evidence indicates that tRNA post-transcriptional modifications may play important roles in complex human pathologies (A. G. Torres et al, Trends. Mol. Med. 20, 306-314 (2014); S. Kirchner, Z. Ignatova, Nat. Rev. Genet. 16, 98-112 (2015); and Pereira, M., et al., 2018. International journal of molecular sciences, 19(12), p. 3738).

Post translational modifications found in tRNAs include τm⁵s²U, 5-taurinomethyl-2-thiouridine; τm⁵U, 5-taurinomethyluridine; Ψ, pseudouridine; ac⁴C, N⁴-acetylcytidine; Am, 2′-O-methyladenosine; Ar(p), 2′-O-ribosyladenosine (phosphate); Cm, 2′-O-methylcytidine; D, dihydrouridine; f⁵C, 5-formylcytidine; Gm, 2′-O-methylguanosine; I, inosine; I⁶A, N⁶-isopentenyladenosine; m¹A, 1-methyladenosine; m¹G, 1-methylguanosine; m¹I, 1-methylinosine; m²G, N²-methylguanosine; m²²G, N²,N²-dimethylguanosine; m³C, 3-methylcytidine; m⁵C, 5-methylcytidine; m⁵U, 5-methyluridine; m⁷G, 7-methylguanosine; mcm⁵s²U, 5-methoxycarbonylmethyl-2-thiouridine; mcm⁵U, 5-methoxycarbonylmethyluridine; ms²A, 2-methylthioadenosine; ms²16A, 2-methylthio-N6-isopentenyladenosine; ms⁶t2A, 2-methylthio-N⁶-threonyl carbamoyladenosine; ncm⁵U, 5-carbamoylmethyluridine; ncm⁵Um, 5-carbamoylmethyl-2′-O-methyluridine; Q, queuosine; rT, ribothymidine; s²U, 2-thiouridine; t⁶A, N⁶-threonylcarbamoyladenosine; Un, 2′-O-methyluridine; yW, wybutosine (S. Kirchner, Z. Ignatova, Nat. Rev. Genet. 16, 98-112 (2015)).

mt-tRNAs may contain one or more of the following post-transcriptional modifications (Positions in a tRNA are according to the nucleotide numbering system from tRNAdb): m¹A9, m¹G9, m²G10, D20, m²G26, m²²G26, Ψ27, Ψ28, Ψ31, m³C32, ω32, f⁵C34, τm⁵U34, S²U34, τm⁵s²U34, Q34, m¹G37, t⁶A37, i⁶A37, ms²A37, Ψ39, Ψ40, m⁵C49, Ψ50, m⁵U54, Ψ55, m¹mA58, Ω67 (T. Suzuki et al, Annu. Rev. Genet. 45, 299-329 (2011)).

Exemplary consensus post-transcriptional modifications for mt-tRNAs are given in Table 5, below. An mt-tRNA may comprise one or more post-transcriptional modification recited in Table 5. Suitably, an mt-tRNA may comprise a nucleotide sequence selected from any of SEQ ID NOs: 1-24 and a corresponding post-transcriptional modification recited in Table 5.

TABLE 5
exemplary mt-tRNA post-transcriptional modifications

| Consensus post- | |
|---|---|
| transcriptional | |
| modification | mt-tRNA species |
| m¹A9 | Lys, Leu(CUN), Pro, Asp |
| m¹G9 | lle, Leu(UUR) |
| m²G10 | Asp, Lys, Leu(UUR), Leu(CUN) |
| m¹A 16 | Arg |
| D20 | Leu(UUR) |
| m²₂G26 | Ile |
| Ψ27 | Asp, Ile, Lys, Met, Leu(UUR), Leu(CUN), Pro |
| Ψ27a | Ser(UCN) |
| Ψ28 | Ile, Lys, Leu(CUN), Pro |
| Ψ31 | Leu(CUN) |
| Ψ32 | Pro |
| f⁵C34 | Met |
| Tm⁵U34 | Leu(UUR) |
| Tm⁵s²U34 | Lys |
| Q34 | Asp |
| m¹G37 | Leu(CUN), Pro |
| t⁶A37 | Ile, Lys, Ser(AGY) |
| Ψ38 | Pro |
| m⁵C48 | Leu(UUR) |
| Ψ50 | Met |
| m⁵U54 | Leu(UUR), Pro |
| Ψ55 | Leu(UUR), Pro |
| m¹A58 | Leu(UUR), Ser(AGY) |

Suitably, the mt-tRNA is a leucine(UUR) mt-tRNA and the post-transcriptional modification is τm⁵U34 or the mt-tRNA is a lysine mt-tRNA and the post-transcriptional modification is τm⁵s²U34.

In some embodiments, the RNA comprises one or more aberrant post-transcriptional modifications, i.e. the RNA is an aberrantly-modified RNA. An “aberrant post-transcriptional modification” is a post-transcriptional modification which differs from a consensus post-transcriptional modification. Suitably, the aberrant post-transcriptional modification is the absence of a consensus post-transcriptional modification.

In some embodiments, the RNA comprises one or more pathogenic aberrant post-transcriptional modification, i.e. the RNA is an aberrantly-modified pathogenic RNA. A “pathogenic aberrant post-transcriptional modification” is an aberrant post-transcriptional modification that increases an individual's susceptibility or predisposition to a certain disease or disorder. Pathogenic aberrant post-transcriptional modifications are described in de Crécy-Lagard, et al., 2019. Nucleic acids research, 47(5), pp. 2143-2159 and Pereira, M., et al., 2018. International journal of molecular sciences, 19(12), p. 3738.

Exemplary aberrant post-transcriptional modifications for mt-tRNAs are given in Table 6, below. An mt-tRNA may comprise one or more aberrant post-transcriptional modification recited in Table 6. Suitably, an mt-tRNA may comprise a nucleotide sequence selected from any of SEQ ID NOs: 1-24 and a corresponding aberrant post-transcriptional modification recited in Table 6.

TABLE 6
exemplary aberrant mt-tRNA post-transcriptional modifications

| Aberrant post- | |
|---|---|
| transcriptional | |
| modification | mt-tRNA species |
| Absence of m¹A9 | Lys, Leu(CUN), Pro, Asp |
| Absence of m¹G9 | Ile, Leu(UUR) |
| Absence of m²G10 | Asp, Lys, Leu(UUR), Leu(CUN) |
| Absence of m¹A16 | Arg |
| Absence of D20 | Leu(UUR) |
| Absence of m²₂G26 | Ile |
| Absence of Ψ27 | Asp, Ile, Lys, Met, Leu(UUR), Leu(CUN), Pro |
| Absence of Ψ27a | Ser(UCN) |
| Absence of Ψ28 | Ile, Lys, Leu(CUN), Pro |
| Absence of Ψ31 | Leu(CUN) |
| Absence of Ψ32 | Pro |
| Absence of f⁵C34 | Met |
| Absence of Tm⁵U34 | Leu(UUR) |
| Absence of Tm⁵s²U34 | Lys |
| Absence of Q34 | Asp |
| Absence of m¹G37 | Leu(CUN), Pro |
| Absence of t⁶A37 | Ile, Lys, Ser(AGY) |
| Absence of 438 | Pro |
| Absence of Ψ⁵C48 | Leu(UUR) |
| Absence of Ψ50 | Met |
| Absence of m⁵U54 | Leu(UUR), Pro |
| Absence of Ψ55 | Leu(UUR), Pro |
| Absence of m¹A58 | Leu(UUR), Ser(AGY) |

In some embodiments:
(i) the RNA is a leucine(UUR) mt-tRNA and the one or more aberrant post-transcriptional modification is the absence of τm⁵U34, m¹G9, m²G10, D20, Ψ27, m⁵C48, m⁵U54, Ψ55, and/or m¹A58;
(ii) the RNA is a lysine mt-tRNA and the one or more aberrant post-transcriptional modifications is the absence of τm⁵s²U34, m¹A9, m²G10, Ψ27, Ψ28, and/or t⁶A37;
(iii) the RNA is a Leu(CUN) mt-tRNA and the one or more aberrant post-transcriptional modification is the absence of m¹A9, m²G10, Ψ27, Ψ28, Ψ31, and/or m¹G37;
(iv) the RNA is a lie mt-tRNA and the one or more aberrant post-transcriptional modification is the absence of m¹G9, m²²G26, Ψ27, Ψ28, and/or t⁶A37;
(v) the RNA is a Asp mt-tRNA and the one or more aberrant post-transcriptional modification is the absence of m¹A9, m²G10, Ψ27, and/or Q34;
(vi) the RNA is a Met mt-tRNA and the one or more aberrant post-transcriptional modification is the absence of Ψ27, f⁵C34, and/or Ψ50;
(vii) the RNA is a Ser(AGY) mt-tRNA and he one or more aberrant post-transcriptional modification is the absence of t⁶A37 and/or m¹A58;
(ix) the RNA is a Pro mt-tRNA and the one or more aberrant post-transcriptional modification is the absence of m¹A9, Ψ27, Ψ28, Ψ32, m¹G37, Ψ38, m⁵U54 and/or Ψ55;
(x) the RNA is a Arg mt-tRNA and the aberrant post-transcriptional modification is the absence of m¹A16; or
(xi) the RNA is a Ser(UCN) mt-tRNA and the aberrant post-transcriptional modification is the absence of Ψ27a.

In some embodiments, the RNA is a leucine(UUR) mt-tRNA and the aberrant post-transcriptional modification is the absence of τm⁵U34 or m¹A58 or the RNA is a lysine mt-tRNA and the aberrant post-transcriptional modification is the absence of τm⁵s²U34. These aberrant modifications are associated with MELAS and MERRF, respectively (Richter, U., et al., 2018. Nature communications, 9(1), pp. 1-11).

In some embodiments, the RNA is a leucine(UUR) mt-tRNA and the aberrant post-transcriptional modification is the absence of τm⁵U34 or the RNA is a lysine mt-tRNA and the aberrant post-transcriptional modification is the absence of τm⁵s²U34.

Padlock Probe

In one aspect, the present invention provides a padlock probe comprising terminal regions complementary to a RNA. The padlock probe may be used to detect and/or quantify said RNA. The RNA may be any RNA described herein.

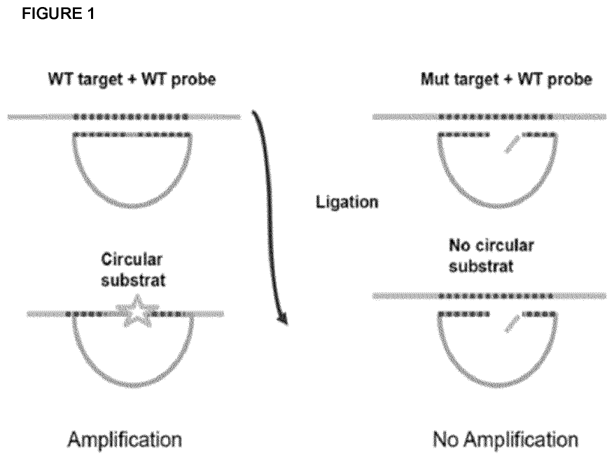

Padlock probes (PLPs) are oligonucleotides, for example single stranded DNA molecules, consisting of two target-complementary segments connected by a linker region. The target-complementary segments are referred to herein as “terminal regions” and are complementary to a “target sequence”. Upon hybridization to the target sequence, the two terminal regions are brought into contact, allowing PLP circularization by ligation (Nilsson, M., et al., 1994. Science, 265(5181), pp. 2085-2088). As used herein, the term “padlock probe” includes molecular inversion probes and connector inversion probes, in which the terminal regions are separated by a gap sequence when hybridised to the target sequence. A schematic showing the hybridization, and circularization of a padlock probe is shown in FIG. 1.

Padlock probes may be used to detect and/or quantify single nucleotide variations in genomic DNA. The circularization of the padlock probe is highly specific as it requires that the terminal regions are brought into contact. Ligation is strongly inhibited by any mismatches at the ligation junction. Multiple padlock probes can be used at once because since only intramolecular probe ligation is scored, cross-reactions are unlikely to arise between many simultaneously added probes. Moreover, highly sensitive detection and/or quantification of circularized probes is possible, e.g. by amplifying reacted probes via a rolling circle replication mechanism.

Techniques for the design and synthesis of padlock probes will be well known to those of skill in the art. The padlock probe may be any length suitable for specific hybridization, circularization, and detection/quantification. Suitably, the padlock probe may be about 50 to about 200 nucleotides in length. Suitably, the padlock probe may be about 50 to about 150 nucleotides in length. Suitably, the padlock probe may be about 50 to about 100 nucleotides in length. Suitably, the padlock probe may be about 70 to about 100 nucleotides in length.

Target Sequence

The terminal regions are each complementary to a region of the RNA. Together the terminal regions are complementary to a target sequence in the RNA. The padlock probe is designed to be complementary to the target sequence and can circularize when the target sequence is present.

The target sequence may be any nucleotide sequence in the RNA which will allow for specific detection/quantification of the RNA. Suitably, the target sequence is a unique nucleotide sequence, e.g. does not occur in any other naturally-occurring RNA (or DNA).

Suitably, the target sequence may be 15-50 nucleotides in length. Suitably, the target sequence may be 20-30 nucleotides in length.

If the RNA is a tRNA comprising a 3′ ligated oligonucleotide, then the target sequence may comprise at least part of the nucleotide sequence of the 3′ ligated oligonucleotide. For example, if the tRNA comprises a sequence comprising or consisting of from 5′ to 3′: SEQ-CCA-(N)ₓ, where x is 1 to 100, 5 to 50, or 10 to 30, the target sequence may comprise at least part of the nucleotide sequence (N)ₓ.

The target sequence may be associated with one or more mutation and/or one or more post-transcriptional modification. The mutation may be any mutation described herein (e.g. in Tables 2-4). The post-transcriptional modification may be any aberrant post-transcriptional modification described herein (e.g. in Table 6).

The target sequence may comprise one or more mutation site and/or one or more post-transcriptional modification site. The mutation site may be the site of any mutation described herein (e.g. in Tables 2-4). The post-transcriptional modification site may be the site of any aberrant post-transcriptional modification described herein (e.g. in Table 6).

The target sequence may comprise one or more mutation and/or one or more post-transcriptional modification. The mutation may be any mutation described herein (e.g. in Tables 2-4). The post-transcriptional modification may be any aberrant post-transcriptional modification described herein (e.g. in Table 6).

Exemplary Target Sequences

The target sequence may consist of a fragment of SEQ ID NOs: 1-24 or a fragment of a variant with at least 90%, at least 95%, or at least 98% sequence identity to any of SEQ ID NOs: 1-24, optionally wherein the target sequence comprises one or more mutations or post-transcriptional modifications described herein (e.g. in Tables 2-4 or 6).

Suitably, the target sequence consists of a fragment of SEQ ID NO: 23 or SEQ ID NO: 24 or a fragment of a variant with at least 90%, at least 95%, or at least 98% sequence identity to SEQ ID NO: 23 or SEQ ID NO: 24, optionally wherein the target sequence comprises one or more mutations or post-transcriptional modifications described herein (e.g. in Tables 2-4 or 6).

In some embodiments, the target sequence consists of a fragment of SEQ ID NO: 23 or a fragment of a variant with at least 90%, at least 95%, or at least 98% sequence identity to SEQ ID NO: 23, wherein the target sequence comprises aberrant post-transcriptional modification U34 and/or A58.

In some embodiments, the target sequence consists of a fragment of SEQ ID NO: 24 or a fragment of a variant with at least 90%, at least 95%, or at least 98% sequence identity to SEQ ID NO: 24, wherein the target sequence comprises aberrant post-transcriptional modification U34.

In some embodiments, the target sequence consists of a fragment of SEQ ID NO: 25 or a fragment of a variant with at least 90%, at least 95%, or at least 98% sequence identity to SEQ ID NO: 25, wherein the target sequence comprises mutation A3243G.

In some embodiments, the target sequence consists of a fragment of SEQ ID NO: 26 or a fragment of a variant with at least 90%, at least 95%, or at least 98% sequence identity to SEQ ID NO: 26, wherein the target sequence comprises mutation T3271C.

In some embodiments, the target sequence consists of a fragment of SEQ ID NO: 27 or a fragment of a variant with at least 90%, at least 95%, or at least 98% sequence identity to SEQ ID NO: 27, wherein the target sequence comprises mutation A8344G.

Terminal Regions

The terminal regions may be any nucleotide sequence which will allow for specific hybridisation and circularization with the target sequence. The terminal regions may be any suitable length. Suitably, the terminal regions are each 5-30 nucleotides in length, optionally 5-15 nucleotides in length. Suitably, the terminal regions in total are 15-50 nucleotides in length, optionally 20-30 nucleotides in length.

Each terminal region is complementary to part of the target sequence. One terminal region (the first terminal region) is complementary to nucleotides a to n of the target sequence, and the other terminal region (the second terminal region) is complementary to nucleotides n+m to b of the target sequence, wherein:
a is the first nucleotide of the target sequence which the first terminal region is complementary to (e.g. a=nucleotide 1 of the target sequence);
n is the last nucleotide of the target sequence which the first terminal region is complementary to (e.g. n=the length of the first terminal region, e.g. n=5 to 30 or n=5 to 15);
n+m is the first nucleotide of the target sequence which the second terminal region is complementary to (e.g. m=1 when the terminal regions are complementary to adjacent regions, e.g. m=2 to 7 when the terminal regions are complementary to non-adjacent regions); and
b is the last nucleotide of the target sequence which the second terminal region is complementary to (e.g. b=the length of the target sequence, e.g. b=15 to 50 or b=2 to 30).

The terminal regions may be complementary to adjacent nucleotide sequences (i.e. m=1). Adjacent nucleotide sequences may together cover the whole target sequence.

The terminal regions may be complementary to non-adjacent nucleotide sequences separated by a gap sequence (i.e. m>1). Non-adjacent nucleotide sequences and the gap sequence may together cover the whole target sequence. Suitably, the gap sequence may consist of 6 or fewer nucleotides (i.e. m≤7), 5 or fewer nucleotides (i.e. m≤6), 4 or fewer nucleotides (i.e. m≤5), 3 or fewer nucleotides (i.e. m≤4), 2 or fewer nucleotides (i.e. m≤3), or one nucleotide (i.e. m≤2). When the terminal regions are complementary to non-adjacent nucleotide sequences separated by a gap sequence the padlock probe may be referred to as a molecular inversion probe and/or a connector inversion probe (Hardenbol, P., et al., 2003. Nature biotechnology, 21(6), pp. 673-678; and Akhras, M. S., et al., 2007. PloS one, 2(9), p. e915).

Suitably, one or more nucleotides in the target sequence may be a mutation site or post-transcriptional modification site. For example, one or more of positions a to n or n+m to b may correspond to a mutation or a post-transcriptional modification in the target sequence. In particular, the terminal nucleotides of a terminal region may be complementary to a mutation site or post-transcriptional modification site to increase specificity of the padlock probe. For example, the nucleotide at position n or the nucleotide at position n+m may correspond to a mutation or a post-transcriptional modification in the target sequence.

Complementarity is achieved by distinct interactions between nucleobases: adenine (A), thymine (T) (or uracil (U) in RNA), guanine (G) and cytosine (C). In nucleic acids, nucleobases are held together by hydrogen bonding, which normally only works efficiently between A and T (or U) and between G and C. A complementary strand of DNA may therefore be constructed based on nucleobase complementarity, where A is complementary to T (or U) and G is complementary to C.

As used herein, the term “complementary” means completely or partially complementary, i.e. each terminal region may be either completely or partially complementary to part of the target sequence. Suitably, each terminal region is completely complementary to part of the target sequence.

As used herein, “completely complementary” refers to the case where every nucleotide is complementary. In other words the terminal region is a (reverse) complement of part of the target sequence.

As used herein, “partially complementary” refers to the case wherein the majority of nucleotides are complementary, but wherein one or more nucleotides are not complementary. In other words, the terminal region is a (reverse) complement of the target sequence, but wherein one or more nucleotides are not complementary. When the terminal regions are partially complementary, the degree of complementarity is such that circularization of the padlock probe can still occur, i.e. the padlock probe can still hybridise to the RNA and ligation can still occur. Suitably, the degree of complementarity is such that the padlock probe can specifically hybridize and circularize, i.e. the padlock probe does not detect other polynucleotides. A person skilled in the art will be able to design partially complementary padlock probes such that the desired degree of complementarity is achieved.

Suitably, when a terminal region is partially complementary, four or fewer nucleotides, three or fewer nucleotides, two or fewer nucleotides, or one nucleotide in the terminal region is not complementary. Suitably, a terminal region may have at least 80% sequence identity, at least 85% sequence identity, at least 90% sequence identity, or at least 95% sequence identity to a fragment of a (reverse) complement of the target sequence.

Suitably, when a terminal region is partially complementary, two or more terminal nucleotides of the terminal region are completely complementary to the target sequence. For example, positions n-x to n and/or positions n+m to n+m+y of the target sequence may be completely complementary to the terminal regions, wherein x and y are each independently 1 or more. Suitably, three or more (x≥2 and/or y≥2), four or more (x≥3 and/or y≥3), or five or more (x≥4 and/or y≥4) nucleotides of the terminal region are completely complementary to the target sequence. This may increase ligation efficiency.

If the RNA is a tRNA comprising a 3′ ligated oligonucleotide, then a terminal region may be complementary to at least part of the nucleotide sequence of the 3′ ligated oligonucleotide. For example, if the tRNA comprises a sequence comprising or consisting of from 5′ to 3′: SEQ-CCA-(N)ₓ, where x is 1 to 100, 5 to 50, or 10 to 30, then a terminal region may be complementary to at least part of the nucleotide sequence (N)ₓ.

A terminal region may be complementary to a nucleotide sequence associated with one or more mutation and/or one or more post-transcriptional modification. The mutation may be any mutation described herein (e.g. in Tables 2-4). The post-transcriptional modification may be any aberrant post-transcriptional modification described herein (e.g. in Table 6).

A terminal region may be complementary to a nucleotide sequence comprising one or more mutation site and/or one or more post-transcriptional modification site. The mutation may be any mutation described herein. The mutation site may be the site of any mutation described herein (e.g. in Tables 2-4). The post-transcriptional modification site may be the site of any aberrant post-transcriptional modification described herein (e.g. in Table 6).

A terminal region may be complementary to a nucleotide sequence comprising one or more mutation and/or one or more post-transcriptional modification. The mutation may be any mutation described herein (e.g. in Tables 2-4). The post-transcriptional modification may be any aberrant post-transcriptional modification described herein (e.g. in Table 6).

Exemplary Terminal Regions

The terminal regions may each consist of a (reverse) complement of a fragment of any one of SEQ ID NOs: 1-24 or a (reverse) complement of a fragment of a variant with at least 90%, at least 95%, or at least 98% sequence identity to any of SEQ ID NOs: 1-24, optionally wherein a terminal region is complementary to a nucleotide sequence comprising one or more mutation site or post-transcriptional modification site described herein (e.g. in Tables 2-4 and 6).

In some embodiments, the terminal regions each consist of a (reverse) complement of a fragment of SEQ ID NO: 23 or SEQ ID NO: 24 or a (reverse) complement of a fragment of a variant with at least 90%, at least 95%, or at least 98% sequence identity to SEQ ID NO: 23 or SEQ ID NO: 24, optionally wherein a terminal region is complementary to a nucleotide sequence comprising one or more mutation site or post-transcriptional modification site described herein (e.g. in Tables 2-4 and 6).

In some embodiments, the terminal regions each consist of a (reverse) complement of a fragment of SEQ ID NO: 23 or a (reverse) complement of a fragment of a variant with at least 90%, at least 95%, or at least 98% sequence identity to SEQ ID NO: 23, wherein one terminal region is complementary to a nucleotide sequence comprising aberrant post-transcriptional modification U34 or A58.

In some embodiments, the terminal regions each consist of a (reverse) complement of a fragment of SEQ ID NO: 24 or a (reverse) complement of a fragment of a variant with at least 90%, at least 95%, or at least 98% sequence identity to SEQ ID NO: 24, wherein one terminal region is complementary to a nucleotide sequence comprising aberrant post-transcriptional modification U34.

In some embodiments, the terminal regions each consist of a (reverse) complement of a fragment of SEQ ID NO: 25 or a (reverse) complement of a fragment of a variant with at least 90%, at least 95%, or at least 98% sequence identity to SEQ ID NO: 25, wherein one terminal region is complementary to a nucleotide sequence comprising mutation A3243G.

In some embodiments, the terminal regions each consist of a (reverse) complement of a fragment of SEQ ID NO: 26 or a (reverse) complement of a fragment of a variant with at least 90%, at least 95%, or at least 98% sequence identity to SEQ ID NO: 26, wherein one terminal region is complementary to a nucleotide sequence comprising mutation T3271C.

In some embodiments, the terminal regions each consist of a (reverse) complement of a fragment of SEQ ID NO: 27 or a (reverse) complement of a fragment of a variant with at least 90%, at least 95%, or at least 98% sequence identity to SEQ ID NO: 27, wherein one terminal region is complementary to a nucleotide sequence comprising mutation A8344G.

Suitably, the terminal regions consist of fragments which are adjacent fragments. Suitably, the terminal regions consist of fragments which are non-adjacent fragments separated by a gap sequence. Suitably, the gap sequence may consist of 6 or fewer nucleotides, 5 or fewer nucleotides, 4 or fewer nucleotides, 3 or fewer nucleotides, 2 or fewer nucleotides, or one nucleotide.

Exemplary reverse complements of SEQ ID NOs: 1-27 are shown in Table 7 below as SEQ ID NOs: 28-54.

TABLE 7
Exemplary reverse complements of consensus
mt-tRNA sequences

| mt-tRNA | Reverse complement |
|---|---|
| Reverse | TGTTAAGAAGAGGAATTGAACCTCTGACTGTAAAGTTT |
| complement | TAAGTTTTATGCGATTACCGGGCTCTGCCATCTTAAC |
| of leucine | |
| (UUR) | |
| mt-tRNA | |
| (SEQ ID | |
| NO: 28) | |
| Reverse | TCACTGTAAAGAGGTGTTGGTTCTCTTAATCTTTAACT |
| complement | TAAAAGGTTAATGCTAAGTTAGCTTTACAGTG |
| of lysine | |
| mt-tRNA | |
| (SEQ ID | |
| NO: 29) | |
| Reverse | TAGTACGGGAAGGGTATAACCAACATTTTCGGGGTATG |
| complement | GGCCCGATAGCTTATTTAGCTGACCTTACT |
| of | |
| methionine | |
| mt-tRNA | |
| (SEQ ID | |
| NO: 30) | |
| Reverse | CAGAAATTAAGTATTGCAACTTACTGAGGGCTTTGAAG |
| complement | GCTCTTGGTCTGTATTTAACCTAAATTTCT |
| of | |
| tryptophan | |
| mt-tRNA | |
| (SEQ ID | |
| NO: 31) | |
| Reverse | TAAGATATATAGGATTTAGCCTATAATTTAACTTTGAC |
| complement | AAAGTTATGAAATGGTTTTTCTAATACCTT |
| of | |
| aspartate | |
| mt-tRNA | |
| (SEQ ID | |
| NO: 32) | |
| Reverse | TAGAAATAAGGGGGTTTAAGCTCCTATTATTTACTCTA |
| complement | TCAAAGTAACTCTTTTATCAGACATATTTCT |
| of | |
| isoleucine | |
| mt-tRNA | |
| (SEQ ID | |
| NO: 33) | |
| Reverse | TACTCTTTTTTGAATGTTGTCAAAACTAGTTAATTGGA |
| complement | AGTTAACGGTACTATTTATACTAAAAGAGT |
| of | |
| glycine | |
| mt-tRNA | |
| (SEQ ID | |
| NO: 34) | |
| Reverse | TTGGTAAATATGATTATCATAATTTAATGAGTCGAAAT |
| complement | CATTCGTTTTGTTTAAACTATATACCA |
| of | |
| arginine | |
| mt-tRNA | |
| (SEQ ID | |
| NO: 35) | |
| Reverse | GGTAAATAAGGGGTCGTAAGCCTCTGTTGTCAGATTCA |
| complement | CAATCTGATGTTTTGGTTAAACTATATTTAC |
| of | |
| histidine | |
| mt-tRNA | |
| (SEQ ID | |
| NO: 36) | |
| Reverse | TGAGAAAGCCATGTTGTTAGACATGGGGGCATGAGTTA |
| complement | GCAGTTCTTGTGAGCTTTCTC |
| of | |
| serine | |
| (AGY) | |
| mt-tRNA | |
| (SEQ ID | |
| NO: 37) | |
| Reverse | TACTTTTATTTGGAGTTGCACCAAAATTTTTGGGGCCT |
| complement | AAGACCAATGGATAGCTGTTATCCTTTAAAAGT |
| of | |
| leucine | |
| (CUN) | |
| mt-tRNA | |
| (SEQ ID | |
| NO: 38) | |
| Reverse | TGTCCTTGGAAAAAGGTTTTCATCTCCGGTTTACAAGA |
| complement | CTGGTGTATTAGTTTATACTACAAGGAC |
| of | |
| threonine | |
| mt-tRNA | |
| (SEQ ID | |
| NO: 39) | |
| Reverse | TGTTTATGGGGTGATGTGAGCCCGTCTAAACATTTTCA |
| complement | GTGTATTGCTTTGAGGAGGTAAGCTACATAAAC |
| of | |
| phenyla- | |
| lanine | |
| mt-tRNA | |
| (SEQ ID | |
| NO: 40) | |
| Reverse | TCAGAGCGGTCAAGTTAAGTTGAAATCTCCTAAGTGTA |
| complement | AGTTGGGTGCTTTGTGTTAAGCTACACTCTG |
| of | |
| valine | |
| mt-tRNA | |
| (SEQ ID | |
| NO: 41) | |
| Reverse | CTAGGACTATGAGAATCGAACCCATCCCTGAGAATCCA |
| complement | AAATTCTCCGTGCCACCTATCACACCCCATCCTA |
| of | |
| glutamine | |
| mt-tRNA | |
| (SEQ ID | |
| NO: 42) | |
| Reverse | TAAGGACTGCAAAACCCCACTCTGCATCAACTGAACGC |
| complement | AAATCAGCCACTTTAATTAAGCTAAGCCCTT |
| of | |
| alanine | |
| mt-tRNA | |
| (SEQ ID | |
| NO: 43) | |
| Reverse | CTAGACCAATGGGACTTAAACCCACAAACACTTAGTTA |
| complement | ACAGCTAAGCACCCTAATCAACTGGCTTCAATCTA |
| of | |
| asparagine | |
| mt-tRNA | |
| (SEQ ID | |
| NO: 44) | |
| Reverse | AAGCCCCGGCAGGTTTGAAGCTGCTTCTTCGAATTTGC |
| complement | AATTCAATATGAAAATCACCTCGGAGCT |
| of | |
| cysteine | |
| mt-tRNA | |
| (SEQ ID | |
| NO: 45) | |
| Reverse | TGGTAAAAAGAGGCCTAACCCCTGTCTTTAGATTTACA |
| complement | GTCCAATGCTTCACTCAGCCATTTTACC |
| of | |
| tyrosine | |
| mt-tRNA | |
| (SEQ ID | |
| NO: 46) | |
| Reverse | CAAAAAAGGAAGGAATCGAACCCCCCAAAGCTGGTTTC |
| complement | AAGCCAACCCCATGGCCTCCATGACTTTTTC |
| of | |
| serine | |
| (UCN) | |
| mt-tRNA | |
| (SEQ ID | |
| NO: 47) | |
| Reverse | TATTCTCGCACGGACTACAACCACGACCAATGATATGA |
| complement | AAAACCATCGTTGTATTTCAACTACAAGAAC |
| of | |
| glutamate | |
| mt-tRNA | |
| (SEQ ID | |
| NO: 48) | |
| Reverse | TCAGAGAAAAAGTCTTTAACTCCACCATTAGCACCCAA |
| complement | AGCTAAGATTCTAATTTAAACTATTCTCTG |
| of | |
| proline | |
| mt-tRNA | |
| (SEQ ID | |
| NO: 49) | |
| Reverse | TGGTGTTAAGAAGAGGAATTGAACCTCTGACTGTAAAG |
| complement | TTTTAAGTTTTATGCGATTACCGGGCCCTGCCATCTTA |
| of | AC |
| mature | |
| leucine | |
| (UUR) | |
| mt-tRNA | |
| (SEQ ID | |
| NO: 50) | |
| Reverse | TGGTCACTGTAAAGAGGTGTTGGTTCTCTTAATCTTTA |
| complement | ACTTAAAAGGTTAATGCTAAGTTAGCTTTACAGTG |
| of | |
| mature | |
| lysine | |
| mt-tRNA | |
| (SEQ ID | |
| NO: 51) | |
| Reverse | TGGTGTTAAGAAGAGGAATTGAACCTCTGACTGTAAAG |
| complement | TTTTAAGTTTTATGCGATTACCGGGCCCTGCCATCTTA |
| of | AC |
| mt-tRNA | |
| Leu (UUR) | |
| with | |
| mutation | |
| A3243G | |
| (SEQ ID | |
| NO: 52) | |
| Reverse | TGTTAAGAAGAGGAATTGAACCTCTGACTGTAAGGTTT |
| complement | TAAGTTTTATGCGATTACCGGGCTCTGCCATCTTAAC |
| of | |
| mt-tRNA | |
| Leu (UUR) | |
| with | |
| mutation | |
| T3271C | |
| (SEQ ID | |
| NO: 53) | |
| Reverse | TGGTCACTGTAAAGAGGTGTTGGCTCTCTTAATCTTTA |
| complement | ACTTAAAAGGTTAATGCTAAGTTAGCTTTACAGTG |
| of | |
| mt-tRNA | |
| Lys with | |
| mutation | |
| A8344G | |
| (SEQ ID | |
| NO: 54) | |

The terminal regions may each consist of a fragment of any one of SEQ ID NOs: 28-51, or a fragment of a variant with at least 90%, at least 95%, or at least 98% sequence identity to any one of SEQ ID NOs: 28-51, optionally wherein a terminal region is complementary to a nucleotide sequence comprising one or more mutation site or post-transcriptional modification site described herein (e.g. in Tables 2-4 and 6).

In some embodiments, the terminal regions each consist of a fragment of SEQ ID NO: 50 or SEQ ID NO: 51, or a fragment of a variant with at least 90%, at least 95%, or at least 98% sequence identity, optionally wherein a terminal region is complementary to a nucleotide sequence comprising one or more mutation site or post-transcriptional modification site described herein (e.g. in Tables 2-4 and 6).

In some embodiments, the terminal regions each consist of a fragment of SEQ ID NO: 50 or a fragment of a variant with at least 90%, at least 95%, or at least 98% sequence identity to SEQ ID NO: 50, wherein a terminal region is complementary to a nucleotide sequence comprising aberrant post-transcriptional modification U34 or A58.

In some embodiments, the terminal regions each consist of a fragment of SEQ ID NO: 51 or a fragment of a variant with at least 90%, at least 95%, or at least 98% sequence identity to SEQ ID NO: 51, wherein a terminal region is complementary to a nucleotide sequence comprising aberrant post-transcriptional modification U34.

In some embodiments, the terminal regions each consist of a fragment of SEQ ID NO: 52 or a fragment of a variant with at least 90%, at least 95%, or at least 98% sequence identity to SEQ ID NO: 52, wherein a terminal region is complementary to a nucleotide sequence comprising mutation A3243G.

In some embodiments, the terminal regions each consist of a fragment of SEQ ID NO: 53 or a fragment of a variant with at least 90%, at least 95%, or at least 98% sequence identity to SEQ ID NO: 53, wherein a terminal region is complementary to a nucleotide sequence comprising mutation T3271C.

In some embodiments, the terminal regions each consist of a fragment of SEQ ID NO: 54 or a fragment of a variant with at least 90%, at least 95%, or at least 98% sequence identity to SEQ ID NO: 54, wherein a terminal region is complementary to a nucleotide sequence comprising mutation A8344G.

Suitably, the terminal regions consist of fragments which are adjacent fragments. Suitably, the terminal regions consist of fragments which are non-adjacent fragments separated by a gap sequence. Suitably, the gap sequence may consist of 6 or fewer nucleotides, 5 or fewer nucleotides, 4 or fewer nucleotides, 3 or fewer nucleotides, 2 or fewer nucleotides, or one nucleotide.

Suitably, when a terminal region is complementary to a mutation or post-transcriptional modification, the complementary nucleotide is a terminal nucleotide, i.e., the nucleotide at position n or the nucleotide at position n+m is complementary to the mutation or a post-transcriptional modification. This may increase the specificity of the padlock probe.

In some embodiments the terminal regions may comprise or consist of SEQ ID NOs: 56 or 58 or variants thereof. Suitably, the variants comprise two or fewer, or one nucleotide substitution. Suitably, the variants do not comprise nucleotide substitutions of the two 3′ terminal nucleotides of SEQ ID NOs: 56 and 58. In some embodiments the terminal regions may consist of SEQ ID NOs: 56 or 58.

In some embodiments the terminal regions may comprise or consist of SEQ ID NOs: 55 and 56 or SEQ ID NOs: 57 and 58 or variants thereof. Suitably, the variants comprise two or fewer, or one nucleotide substitution. Suitably, the variants do not comprise nucleotide substitutions of the two 5′ terminal nucleotides of SEQ ID NOs: 55 and 57 and the two 3′ terminal nucleotides of SEQ ID NOs: 56 and 58.

In some embodiments the terminal regions may consist of SEQ ID NOs: 55 and 56 or SEQ ID NOs: 57 and 58.

Exemplary terminal regions for Leu (UUR)
mt-tRNA with mutation A3243G
(SEQ ID NOs: 55 and 56)
CTGCCATCTTAAC and TTACCGGGCC
Exemplary terminal regions for Leu (UUR)
mt-tRNA with wild-type A3243
(SEQ ID NOs: 57 and 58)
CTGCCATCTTAAC and TTACCGGGCT

The nucleotides shown in bold in SEQ ID NOs: 56 and 58 are those nucleotides corresponding to mutation site A3243.

In some embodiments the terminal regions may comprise or consist of SEQ ID NOs: 59 and 60 or SEQ ID NOs: 61 and 62 or variants thereof. Suitably, the variants comprise two or fewer, or one nucleotide substitution. Suitably, the variants do not comprise nucleotide substitutions of the two 5′ terminal nucleotides of SEQ ID NOs: 59 and 61 and the two 3′ terminal nucleotides of SEQ ID NOs: 60 and 62.

In some embodiments the terminal regions may consist of SEQ ID NOs: 59 and 60 or SEQ ID NOs: 61 and 62.

Exemplary terminal regions for initiator
methionine nuclear tRNA
(SEQ ID NOs: 59 and 60)
ATGGGCCCAGCACGCTTC and CATCGACCTCTGGGTT
Exemplary terminal regions for methionine mt-tRNA
(SEQ ID NOs: 61 and 62)
AGCTTATTTAGCTGACCTTACT and ATTTTCGGGGTATGGGCCCGAT

Linker Region

The linker region may be any linker regions which allows for detection and/or quantification of the circularized padlock probe. The linker regions may be any suitable length. Suitably, the linker region may be 10-150 nucleotides in length. Suitably, the linker region may be 10-100 nucleotides in length. Suitably, the linker region may be 10-60 nucleotides in length.

The linker region will typically comprise one or more primer binding sites for detection and/or quantification via amplification of the circularized padlock probe. As used herein, a “primer binding site” is a nucleotide sequence where an RNA or DNA single-stranded primer can bind to start replication or the reverse complement thereof. A primer binding site may consist of any suitable nucleotide sequence for specific amplification. Suitably, a primer binding site is 10-30 nucleotides in length.

The linker region may comprise a forward primer binding site. A “forward primer site” is complementary to a forward primer. The forward primer may bind to the circularized padlock probe at the forward primer site. When the forward primer is extended by a DNA polymerase it may form a complementary strand to the circularized padlock probe. By using a forward primer binding site, the circularized padlock probe may be amplified by rolling circle amplification.

The linker region may further comprise a reverse primer binding site. A “reverse primer site” is identical to a reverse primer, i.e. the complement of the “reverse primer site” is complementary to a reverse primer. The reverse primer may bind to the complementary strand of the circularized padlock probe (e.g. formed by extension of the forward primer) at the site corresponding to the reverse primer site. When the reverse primer is extended by a DNA polymerase it may form a strand identical to the circularized padlock probe. By using a forward primer binding site and a reverse primer binding site, the circularized padlock probe may be amplified by hyper-branched rolling circle amplification.

The linker region may comprise a tag or barcode sequence. A tag or barcode sequence may be used to detect and/or quantify the circularized padlock probe. For example, a tag or barcode can be used to hybridize circularized (and optionally amplified) padlock probes to a microarray. Incorporating a tag or barcode sequence may allow the detection and/or quantification to be multiplexed. A method for tagged hyper-branched rolling circle amplification is described in US2012264630A1. A tag or barcode sequence may consist of any sequence suitable for specific detection and/or quantification. Suitably, a tag or barcode sequence is 10-30 nucleotides in length.

The padlock probe may comprise or consist of from 5′ to 3′: a first terminal region; a first primer binding site; a second primer binding site; and a second terminal region. Suitably, the first primer binding site is a forward primer site and the second primer binding site is a reverse primer binding site, or vice versa. Optionally, the padlock probe further comprises a tag or barcode sequence.

The terminal regions, primer sites, and optionally tag or barcode sequence may overlap as long as the padlock probe is still capable of circularising and being detected. Suitably, the overlap may be five or fewer, four or fewer, three of fewer, two or fewer, or one or fewer nucleotides. Suitably, the terminal regions, primer sites, and optionally tag or barcode sequence do not overlap.

Any suitable nucleotide sequence may be present between the terminal regions, primer sites, and optionally tag or barcode sequence, e.g. a nucleotide sequence with one or more, two or more, three or more, four or more, or five or more nucleotides, as long as the padlock probe is still capable of circularising and being detected. For example, the nucleotide sequence (A)_{z} may be inserted, where z is between 1 and 5.

Exemplary Padlock Probes

In some embodiments, the padlock probe comprises or consists of a nucleotide sequence with at least 70%, at least 80%, at least 85%, at least 90%, at least 95%, at least 98%, or at least 99% sequence identity to any one of SEQ ID NOs: 63-66, or a fragment thereof. In some embodiments, the padlock probe comprises or consists of the nucleotide sequence of any one of SEQ ID NOs: 63-66, or a fragment thereof.

In some embodiments, the padlock probe comprises or consists of a nucleotide sequence with at least 70%, at least 80%, at least 85%, at least 90%, at least 95%, at least 98%, or at least 99% sequence identity to SEQ ID NO: 63 or SEQ ID NO: 64, or a fragment thereof. In some embodiments, the padlock probe comprises or consists of the nucleotide sequence of SEQ ID NO: 63 or SEQ ID NO: 64, or a fragment thereof.

Exemplary padlock probe for Leu (UUR) mt-tRNA
with mutation A3243G
(SEQ ID NO: 63)
CTGCCATCTTAACTGTAGGGGAGGAGCTGACCACACTTATCATTATCTT
CGCACAGACGTTACCGGGCC
Exemplary padlock probe for Leu (UUR) mt-tRNA
with wild-type A3243
(SEQ ID NO: 64)
CTGCCATCTTAACTGGTGAGGTAAAGAACGAAGACTTCTTATCATTATC
TACGAGCGGACGATTACCGGGCT
Exemplary initiator methionine nuclear tRNA
padlock probe
(SEQ ID NO: 65)
ATGGGCCCAGCACGCTTCTGAAATCTTGTAGCAGGACTCGTGACAGGCA
AGGAATACAGGCATCGACCTCTGGGTT
Exemplary methionine mt-tRNA padlock probe
(SEQ ID NO: 66)
AGCTTATTTAGCTGACCTTACTGTATGGATCGTGCCTTGTCATCGTGGA
CGCCAGAAAATTAAGATTTTCGGGGTATGGGCCCGAT

Kit, Composition, and RNA Detection and/or Quantification System

The present invention provides a kit, composition, or RNA detection and/or quantification system comprising one or more padlock probes of the present invention.

As used herein, an “RNA detection and/or quantification system” is a system which comprises all components necessary to detect and/or quantify an RNA using a padlock probe.

Padlock Probe Combinations

In some embodiments, the kit, composition, or RNA detection and/or quantification system of the present invention comprises two or more different padlock probes. A combination of two or more different padlock probes may be useful, for example, to detect and/or quantify both mutant RNA and non-mutant RNA, or to detect and/or quantify an RNA of interest and a reference RNA.

Suitably, when the kit, composition, or RNA detection and/or quantification system of the present invention comprises two or more different padlock probes, each different padlock probe may comprise a unique tag or barcode sequence. The unique tag or barcode sequence may be used for multiplexed detection and/or quantification.

Detection and/or Quantification of Mutant RNA and Non-Mutant RNA

The kit, composition, or RNA detection and/or quantification system may comprise one or more padlock probes for detecting and/or quantifying a mutant RNA and one or more padlock probes for detecting and/or quantifying a non-mutant (e.g. consensus) RNA.

Suitably, the mutant RNA is identical to the non-mutant (e.g. consensus) RNA except for the mutation. The RNA and the mutation may be any RNA and mutation described herein.

In some embodiments, the kit, composition, or RNA detection and/or quantification system comprises:
(i) a first padlock probe comprising terminal regions complementary to a mutant RNA; and
(ii) a second padlock probe comprising terminal regions complementary to a non-mutant (e.g. consensus) RNA.

In some embodiments:
(a) the first padlock probe comprises terminal regions complementary to a mutant mt-tRNA Leu (UUR) with mutation A3243G, and the second padlock probe comprises terminal regions complementary to a non-mutant (e.g. consensus) mt-tRNA Leu (UUR);
(b) the first padlock probe comprises terminal regions complementary to a mutant mt-tRNA Leu (UUR) with mutation T3271C, and the second padlock probe comprises terminal regions complementary to a non-mutant (e.g. consensus) mt-tRNA Leu (UUR); or
(c) the first padlock probe comprises terminal regions complementary to a mutant mt-tRNA Lys with mutation A8344G, and the second padlock probe comprises terminal regions complementary to a non-mutant (e.g. consensus) mt-tRNA Lys.

In some embodiments, the first padlock probe comprises terminal regions complementary to a mutant mt-tRNA Leu (UUR) with mutation A3243G, and the second padlock probe comprises terminal regions complementary to a non-mutant (e.g. consensus) mt-tRNA Leu (UUR).

In some embodiments, the first padlock probe comprises terminal regions consisting of SEQ ID NOs: 55 and 56, and the second padlock probe comprises terminal regions consisting of SEQ ID NOs: 57 and 58.

In some embodiments, the first padlock probe comprises or consists of the nucleotide sequence of SEQ ID NO: 63 or a variant with at least 70%, at least 80%, at least 85%, at least 90%, at least 95%, at least 98%, or at least 99% sequence identity to SEQ ID NO: 63, and the second padlock probe comprises or consists of the nucleotide sequence of SEQ ID NO: 64 or a variant with at least 70%, at least 80%, at least 85%, at least 90%, at least 95%, at least 98%, or at least 99% sequence identity to SEQ ID NO: 64.

In some embodiments, the first padlock probe consists of the nucleotide sequence of SEQ ID NO: 63, and the second padlock probe consists of the nucleotide sequence of SEQ ID NO: 64.

Detection and/or Quantification of Corresponding Genes

The kit, composition, or RNA detection and/or quantification system may comprise one or more padlock probes for detecting and/or quantifying an RNA of interest and one or more padlock probes for detecting and/or quantifying the gene which encodes the RNA of interest. For example, if the RNA of interest is a heteroplasmic mutant RNA, the RNA mutation load and DNA mutation load can be compared.

The padlock probes for detecting and/or quantifying the gene which encodes the RNA of interest is not a padlock probe according to the present invention. However, such padlock probes can be designed and synthesised by one of skill in the art.

In some embodiments, the kit, composition, or RNA detection and/or quantification system comprises:
(i) a padlock probe comprising terminal regions complementary to a RNA of interest; and
(ii) a padlock probe comprising terminal regions complementary to the gene which encodes the RNA of interest.

In some embodiments, the kit, composition, or RNA detection and/or quantification system comprises:
(i) a first padlock probe comprising terminal regions complementary to a mutant RNA; and/or
(ii) a second padlock probe comprising terminal regions complementary to a non-mutant (e.g. consensus) RNA; and/or
(iii) a third padlock probe comprising terminal regions complementary to a gene encoding said mutant RNA; and/or
(iv) a fourth padlock probe comprising terminal regions complementary to a gene encoding said non-mutant (e.g. consensus) RNA.

Suitably, the kit, composition, or RNA detection and/or quantification system comprises the first and third padlock probes and/or the second and fourth padlock probes. Suitably, the kit, composition or RNA detection and/or quantification system comprises the first, second, third, and fourth padlock probes.

In some embodiments:
(a) the first padlock probe comprises terminal regions complementary to a mutant mt-tRNA Leu (UUR) with mutation A3243G, the second padlock probe comprises terminal regions complementary to a non-mutant (e.g. consensus) mt-tRNA Leu (UUR), the third padlock probe comprises terminal regions complementary to a gene encoding the mutant mt-tRNA Leu (UUR) with mutation A3243G, and the fourth padlock probe comprises terminal regions complementary to a gene encoding the non-mutant (e.g. consensus) mt-tRNA Leu (UUR);
(b) the first padlock probe comprises terminal regions complementary to a mutant mt-tRNA Leu (UUR) with mutation T3271C, the second padlock probe comprises terminal regions complementary to a non-mutant (e.g. consensus) mt-tRNA Leu (UUR), the third padlock probe comprises terminal regions complementary to a gene encoding the mutant mt-tRNA Leu (UUR) with mutation T3271C, and the fourth padlock probe comprises terminal regions complementary to a gene encoding the non-mutant (e.g. consensus) mt-tRNA Leu (UUR); or
(c) the first padlock probe comprises terminal regions complementary to a mutant mt-tRNA Lys with mutation A8344G, the second padlock probe comprises terminal regions complementary to a non-mutant (e.g. consensus) mt-tRNA Lys, the third padlock probe comprises terminal regions complementary to a gene encoding the mutant mt-tRNA Lys with mutation A8344G, and the fourth padlock probe comprises terminal regions complementary to a gene encoding the non-mutant (e.g. consensus) mt-tRNA Lys.

In some embodiments, the first padlock probe comprises terminal regions complementary to a mutant mt-tRNA Leu (UUR) with mutation A3243G, the second padlock probe comprises terminal regions complementary to a non-mutant (e.g. consensus) mt-tRNA Leu (UUR), the third padlock probe comprises terminal regions complementary to a gene encoding the mutant mt-tRNA Leu (UUR) with mutation A3243G, and the fourth padlock probe comprises terminal regions complementary to a gene encoding the non-mutant (e.g. consensus) mt-tRNA Leu (UUR).

In some embodiments, the first padlock probe comprises terminal regions consisting of SEQ ID NOs: 55 and 56, the second padlock probe comprises terminal regions consisting of SEQ ID NOs: 57 and 58, the third padlock probe comprises terminal regions consisting of SEQ ID NOs: 67 and 68, and the fourth padlock probe comprises terminal regions consisting of SEQ ID NOs: 69 and 70.

In some embodiments, the first padlock probe comprises or consists of the nucleotide sequence of SEQ ID NO: 63 or a variant with at least 70%, at least 80%, at least 85%, at least 90%, at least 95%, at least 98%, or at least 99% sequence identity to SEQ ID NO: 63, the second padlock probe comprises or consists of the nucleotide sequence of SEQ ID NO: 64 or a variant with at least 70%, at least 80%, at least 85%, at least 90%, at least 95%, at least 98%, or at least 99% sequence identity to SEQ ID NO: 64, the third padlock probe comprises or consists of the nucleotide sequence of SEQ ID NO: 71 or a variant with at least 70%, at least 80%, at least 85%, at least 90%, at least 95%, at least 98%, or at least 99% sequence identity to SEQ ID NO: 71, and the fourth padlock probe comprises or consists of the nucleotide sequence of SEQ ID NO: 72 or a variant with at least 70%, at least 80%, at least 85%, at least 90%, at least 95%, at least 98%, or at least 99% sequence identity to SEQ ID NO: 72.

In some embodiments, the first padlock probe consists of the nucleotide sequence of SEQ ID NO: 63, the second padlock probe consists of the nucleotide sequence of SEQ ID NO: 64, the third padlock probe consists of the nucleotide sequence of SEQ ID NO: 71, and the fourth padlock probe consists of the nucleotide sequence of SEQ ID NO: 72.

Exemplary terminal regions for gene encoding
Leu (UUR) mt-tRNA with mutation A3243G
(SEQ ID NOs: 67 and 68)
GCCCGGTAATCGCATAAAACTTAAAACT and TTAAGATGGCAG
Exemplary terminal regions for gene encoding
Leu (UUR) mt-tRNA with wild-type A3243G
(SEQ ID NOs: 69 and 70)
GCCCGGTAATCGCATAAAACTTAAAACT and TTAAGATGGCAGA
Exemplary padlock probe for gene encoding Leu
(UUR) mt-tRNA with mutation A3243G
(SEQ ID NO: 71)
GCCCGGTAATCGCATAAAACTTAAAACTAAAGATTGAGAGAGTTTGGAA
GTGTCTGTCCAGGGATCTGCTCTTAAAATTAAGATGGCAG
Exemplary padlock probe for gene encoding Leu
(UUR) mt-tRNA with wild-type A3243G
(SEQ ID NO: 72)
GCCCGGTAATCGCATAAAACTTAAAACTAAATGAAATCTTGTAGCAGGA
CTCTGGACAGGCAAGGAATACAGGAAATTAAGATGGCAGA

Detection and/or Quantification of Reference RNAs

The kit, composition, or RNA detection and/or quantification system may comprise one or more padlock probes for detecting and/or quantifying an RNA of interest, and one or more padlock probes for detecting and/or quantifying a reference RNA. The quantity of a reference tRNA may, for example, act as indicator of the level of transcriptional activity.

In some embodiments, the kit, composition, or RNA detection and/or quantification system comprises:
(i) a padlock probe comprising terminal regions complementary to a RNA of interest; and
(ii) a padlock probe comprising terminal regions complementary to a reference RNA.

In some embodiments, the kit, composition, or RNA detection and/or quantification system comprises:
(i) a first padlock probe comprising terminal regions complementary to a mutant RNA; and/or
(ii) a second padlock probe comprising terminal regions complementary to a non-mutant (e.g. consensus) RNA; and/or
(iii) a third padlock probe comprising terminal regions complementary to a gene encoding said mutant RNA; and/or
(iv) a fourth padlock probe comprising terminal regions complementary to a gene encoding said non-mutant (e.g. consensus) RNA; and/or
(v) a fifth padlock probe comprising terminal regions complementary to a reference RNA.

Suitably, the kit, composition, or RNA detection and/or quantification system comprise any combination of the first, second, third, fourth, and fifth padlock probes. Suitably, the kit, composition or RNA detection and/or quantification system comprises the first, second, and fifth padlock probes. Suitably, the kit, composition or RNA detection and/or quantification system comprises the first, second, third, fourth, and fifth padlock probes.

The kit, composition, or RNA detection and/or quantification system may comprise one or more padlock probes for detecting and/or quantifying an RNA of interest, and one or more padlock probes for detecting and/or quantifying a reference nuclear-encoded RNA and/or one or more padlock probes for detecting and/or quantifying a reference mitochondrial-encoded RNA.

In some embodiments, the kit, composition, or RNA detection and/or quantification system comprises:
(i) a padlock probe comprising terminal regions complementary to a RNA of interest; and
(ii) a padlock probe comprising terminal regions complementary to a reference nuclear-encoded RNA and/or a padlock probe comprising terminal regions complementary to a reference mitochondrially-encoded RNA.

In some embodiments, the kit, composition, or RNA detection and/or quantification system comprises:
(i) a first padlock probe comprising terminal regions complementary to a mutant RNA; and/or
(ii) a second padlock probe comprising terminal regions complementary to a non-mutant (e.g. consensus) RNA; and/or
(iii) a third padlock probe comprising terminal regions complementary to a gene encoding said mutant RNA; and/or
(iv) a fourth padlock probe comprising terminal regions complementary to a gene encoding said non-mutant (e.g. consensus) RNA; and/or
(v) a fifth padlock probe comprising terminal regions complementary to a reference nuclear-encoded RNA; and/or
(vi) a sixth padlock probe comprising terminal regions complementary to a reference mitochondrially-encoded RNA.

Suitably, the kit, composition, or RNA detection and/or quantification system comprises any combination of the first, second, third, fourth, fifth, and sixth padlock probes. Suitably, the kit, composition or RNA detection and/or quantification system comprises the first, second, and sixth padlock probes. Suitably, the kit, composition or RNA detection and/or quantification system comprises the first, second, third, fourth, fifth, and sixth padlock probes.

In some embodiments, the fifth padlock probe comprises terminal regions complementary to initiator methionine nuclear-encoded tRNA. Suitably, the fifth padlock probe comprises terminal regions consisting of SEQ ID NOs: 59 and 60. Suitably, the fifth padlock probe comprises or consists of the nucleotide sequence of SEQ ID NO: 65 or a variant with at least 70%, at least 80%, at least 85%, at least 90%, at least 95%, at least 98%, or at least 99% sequence identity to SEQ ID NO: 65.

In some embodiments, the sixth padlock probe comprises terminal regions complementary to methionine mt-tRNA. Suitably, the sixth padlock probe comprises terminal regions consisting of SEQ ID NOs: 61 and 62. Suitably, the sixth padlock probe comprises or consists of the nucleotide sequence of SEQ ID NO: 66 or a variant with at least 70%, at least 80%, at least 85%, at least 90%, at least 95%, at least 98%, or at least 99% sequence identity to SEQ ID NO: 66.

Multiplexed Detection and/or Quantification

The kit, composition, or RNA detection and/or quantification system may comprise a plurality of padlock probes for detecting and/or quantifying a plurality of RNAs of interest. Suitably, each padlock probe comprises a unique tag or barcode sequence for specific detection and/or quantification. Suitably, each padlock probe is specific for a unique target sequence.

The unique target sequences may be in different RNAs. For example, the kit, composition, or RNA detection and/or quantification system suitably comprises:
(a) a first padlock probe comprising terminal regions complementary to a first RNA and a first tag or barcode sequence; and
(b) a second padlock probe comprising terminal regions complementary to a second RNA and a second tag or barcode sequence.

The unique target sequences may be in the same RNA. For example, the kit, composition, or RNA detection and/or quantification system suitably comprises:
(a) a first padlock probe comprising terminal regions complementary to a first target sequence on a RNA and a first tag or barcode sequence; and
(b) a second padlock probe comprising terminal regions complementary to a second target sequence on said RNA and a second tag or barcode sequence.

In some embodiments:
(a) the first padlock probe comprising terminal regions complementary to a mutant mt-tRNA Leu (UUR) with mutation A3243G and a first tag or barcode sequence; and the second padlock probe comprising terminal regions complementary to a mutant mt-tRNA Leu (UUR) with mutation T3271C and a second tag or barcode sequence;
(b) the first padlock probe comprises terminal regions complementary to a mutant mt-tRNA Leu (UUR) with mutation A3243G and a first tag or barcode sequence; and the second padlock probe comprises terminal regions complementary to a non-mutant (e.g. consensus) mt-tRNA Leu (UUR) and a second tag or barcode sequence;
(c) the first padlock probe comprises terminal regions complementary to a mutant mt-tRNA Leu (UUR) with mutation T3271C and a first tag or barcode sequence; and the second padlock probe comprises terminal regions complementary to a non-mutant (e.g. consensus) mt-tRNA Leu (UUR) and a second tag or barcode sequence; or
(d) the first padlock probe comprises terminal regions complementary to a mutant mt-tRNA Lys with mutation A8344G and a first tag or barcode sequence; and the second padlock probe comprises terminal regions complementary to a non-mutant (e.g. consensus) mt-tRNA Lys and a second tag or barcode sequence.

Additional Components

The kit, composition, or RNA detection and/or quantification system may comprise any additional component, for example any additional component necessary to detect and/or quantify an RNA using a padlock probe of the present invention.

Primers

The kit, composition, or RNA detection and/or quantification system may comprise one or more primers. A “primer” is a short oligonucleotide that provides a starting point for DNA synthesis. The primer may be any suitable primer. Suitably, the one or more primers are complementary to the one or more primer binding sites present in the one or more padlock probes of the present invention which are present in the kit, composition, or RNA detection and/or quantification system.

In some embodiments the kit, composition, or RNA detection and/or quantification system of the present invention comprises: (i) one or more padlock probes of the invention comprising or consisting of from 5′ to 3′: a first terminal region; a first primer binding site; a second primer binding site; and a second terminal region; (ii) one or more first primers; and (iii) one or more second primers. Suitably, the first primer is a forward primer and the second primer is a reverse primer, or vice versa.

For example, the kit, composition, or RNA detection and/or quantification system of the present invention may comprise:
(a) a padlock probe consisting of the nucleotide sequence of SEQ ID NO: 63, a forward primer consisting of the nucleotide sequence of SEQ ID NO: 73, and a reverse primer consisting of the nucleotide sequence of SEQ ID NO: 74;
(b) a padlock probe consisting of the nucleotide sequence of SEQ ID NO: 64, a forward primer consisting of the nucleotide sequence of SEQ ID NO: 75, and a reverse primer consisting of the nucleotide sequence of SEQ ID NO: 76;
(c) a padlock probe consisting of the nucleotide sequence of SEQ ID NO: 65, a forward primer consisting of the nucleotide sequence of SEQ ID NO: 77, and a reverse primer consisting of the nucleotide sequence of SEQ ID NO: 78; and/or
(d) a padlock probe consisting of the nucleotide sequence of SEQ ID NO: 66, a forward primer consisting of the nucleotide sequence of SEQ ID NO: 79, and a reverse primer consisting of the nucleotide sequence of SEQ ID NO: 80.

Exemplary primers for a padlock probe
consisting of SEQ ID NO: 63:
Forward primer
(SEQ ID NO: 73)
TGGTCAGCTCCTCCCCTACA
Reverse primer
(SEQ ID NO: 74)
CACTTATCATTATCTTCGCACAGACG
Exemplary primers for a padlock probe
consisting of SEQ ID NO: 64:
Forward primer
(SEQ ID NO: 75)
GAAGTCTTCGTTCTTTACCTCACCA
Reverse primer
(SEQ ID NO: 76)
TTATCATTATCTACGAGCGGACGA
Exemplary primers for a padlock probe
consisting of SEQ ID NO: 65:
Forward primer
(SEQ ID NO: 77)
GAGTCCTGCTACAAGATTTCA
Reverse primer
(SEQ ID NO: 78)
TGACAGGCAAGGAATACAGG
Exemplary primers for a padlock probe
consisting of SEQ ID NO: 66:
Forward primer
(SEQ ID NO: 79)
ATGACAAGGCACGATCCATAC
Reverse primer
(SEQ ID NO: 80)
CGTGGACGCCAGAAAATTAAG

Suitably, one or more of the primers are primers according to US2012264630A1. For example, one or more the primers may consist of or comprise the following sequence, from its 5′ end to its 3′ end: 5′-(F1)ₙ₁-T1-(E1)ₘ₁-A1-3′, in which:
F1 represents a terminal group selected from a tag and a coupling agent;
T1 represents a barcode nucleotide sequence, optionally consisting of 6 to 30 nucleotides;
E1 represents a spacer that blocks polymerization of the strand complementary to said nucleotide sequence T1 by a DNA polymerase deprived of exonuclease activity and having a strand displacement activity;
A1 represents a nucleotide sequence, optionally consisting of 10 to 40 nucleotides, that is capable of hybridizing with a primer site; and
n1 and m1 are independently a whole number equal to 0 or 1.

Suitably, one or more of the forward primers consists of or comprises the following sequence, from its 5′ end to its 3′ end: 5′-(F1)ₙ₁-T1-(E1)ₘ₁-A1-3′, in which:
F1 represents a terminal group selected from a tag and a coupling agent;
T1 represents a barcode nucleotide sequence, optionally consisting of 6 to 30 nucleotides;
E1 represents a spacer that blocks polymerization of the strand complementary to said nucleotide sequence T1 by a DNA polymerase deprived of exonuclease activity and having a strand displacement activity;
A1 represents a nucleotide sequence, optionally consisting of 10 to 40 nucleotides, that is capable of hybridizing with a forward primer site; and
n1 and m1 are independently a whole number equal to 0 or 1; and/or
one or more of the reverse primers consists of or comprises the following sequence, from its 5′ end to its 3′ end: 5′-(F2)ₙ₂-T2-(E2)ₘ₂-A2-3′, in which:
F2 represents a terminal group selected from a tag and a coupling agent, which may be identical to or different from the terminal group F1;
T2 represents a barcode nucleotide sequence, optionally consisting of 6 to 30 nucleotides, which may be identical to or different from the nucleotide sequence T1;
E2 represents a spacer that blocks polymerization of the strand complementary to said nucleotide sequence T2 by a DNA polymerase deprived of exonuclease activity and having a strand displacement activity, and which may be identical to or different from the spacer E1;
A2 represents a nucleotide sequence, optionally consisting of 10 to 40 nucleotides, that is capable of hybridizing with a reverse primer site; and
n2 and m2 are independently a whole number equal to 0 or 1;

Suitably, m1+m2 is equal to 1 or 2. Suitably, the spacer E1 and/or E2 is selected from the group constituted by an abasic site and a linear or branched, optionally substituted alkyl, alkenyl or alkynyl group, e.g. polyethylene glycol. Suitably, said tag is selected from the group constituted by a luminescent agent, a radioisotope, an enzyme, biotin, acrylamide, a thiol and a phosphorothioate.

Capture Probes

The kit, composition, or RNA detection and/or quantification system may comprise one or more capture probes. A “capture probe” is an oligonucleotide which can be used to specifically purify a RNA or DNA target molecule. The one or more capture probes may be any suitable capture probes. The one or more capture probes may specifically hybridise to one or more padlock probes of the invention. Suitably, the one or more capture probes are complementary to a nucleotide sequence of 10-50 nucleotides in one or more padlock probes of the invention.

Suitably, the one or more capture probes are modified oligonucleotides. Suitably, the modification can be used to specifically purify the RNA or DNA target molecule. Suitably, the capture probe is a biotinylated oligonucleotide.

The kit, composition, or RNA detection and/or quantification system may comprise one beads specific for the capture probe. For example, if the capture probe is a biotinylated oligonucleotide, then the beads may be coated with streptavidin. Suitably, the beads are magnetic beads.

For example, the kit, composition, or RNA detection and/or quantification system of the present invention may comprise:
(a) a padlock probe consisting of the nucleotide sequence of SEQ ID NO: 63 and a capture probe consisting of the nucleotide sequence of SEQ ID NO: 81, and optionally a forward primer consisting of the nucleotide sequence of SEQ ID NO: 73, and a reverse primer consisting of the nucleotide sequence of SEQ ID NO: 74;
(b) a padlock probe consisting of the nucleotide sequence of SEQ ID NO: 64 and a capture probe consisting of the nucleotide sequence of SEQ ID NO: 82, and optionally a forward primer consisting of the nucleotide sequence of SEQ ID NO: 75 and a reverse primer consisting of the nucleotide sequence of SEQ ID NO: 76;
(c) a padlock probe consisting of the nucleotide sequence of SEQ ID NO: 65 and a capture probe consisting of the nucleotide sequence of SEQ ID NO: 83, and optionally a forward primer consisting of the nucleotide sequence of SEQ ID NO: 77 and a reverse primer consisting of the nucleotide sequence of SEQ ID NO: 78; and/or
(d) a padlock probe consisting of the nucleotide sequence of SEQ ID NO: 66 and a capture probe consisting of the nucleotide sequence of SEQ ID NO: 84, and optionally a forward primer consisting of the nucleotide sequence of SEQ ID NO: 79 and a reverse primer consisting of the nucleotide sequence of SEQ ID NO: 80.

Exemplary capture probe for a padlock probe consisting of SEQ ID NO: 63
(SEQ ID NO: 81):
Biotin 5′TGG-TGT-TAA-GAA-GAG-GAA-TTG-AAC-CTC-T 3′ Spacer C3
Exemplary capture probe for a padlock probe consisting of SEQ ID NO: 64
(SEQ ID NO: 82):
Biotin 5′TGG-TGT-TAA-GAA-GAG-GAA-TTG-AAC-CTC-T 3′ Spacer C3
Exemplary capture probe for a padlock probe consisting of SEQ ID NO: 65
(SEQ ID NO: 83):
Biotin 5′CTGCGCCACTCTGCT 3′ Spacer C3
Exemplary capture probe for a padlock probe consisting of SEQ ID NO: 66
(SEQ ID NO: 84):
Biotin TAG-TAC-GGG-AAG-GGT-AT SpacerC3

DNA Ligase

The kit, composition, or RNA detection and/or quantification system may comprise one or more DNA ligases. A “DNA ligase” is an enzyme that facilitates the joining of DNA strands together by catalyzing the formation of a phosphodiester bond. The DNA ligase may be any suitable DNA ligase. Suitably, the DNA ligase can efficiently ligate the padlock probes of the invention when they are hybridised to the RNA.

A suitable DNA ligase is a Splint R ligase. Splint R Ligase (NCBI Reference Sequence: NP_048900.1) is also known as PBCV-1 DNA Ligase or Chlorella virus DNA and efficiently catalyzes the ligation of adjacent, single-stranded DNA splinted by a complementary RNA strand (Jin, J., et al., 2016. Nucleic acids research, 44(13), pp. e116-e116).

Suitably, a Splint R ligase comprises or consists of the amino acid sequence of SEQ ID NO: 85 or a variant with at least 70%, at least 80%, at least 85%, at least 90%, at least 95%, at least 98%, or at least 99% sequence identity to SEQ ID NO: 85.

Exemplary Splint R ligase
(SEQ ID NO: 85)
MAITKPLLAATLENIEDVQFPCLATPKIDGIRSVKQTQMLSRTFKPIRN
SVMNRLLTELLPEGSDGEISIEGATFQDTTSAVMTGHKMYNAKFSYYWF
DYVTDDPLKKYIDRVEDMKNYITVHPHILEHAQVKIIPLIPVEINNITE
LLQYERDVLSKGFEGVMIRKPDGKYKFGRSTLKEGILLKMKQFKDAEAT
IISMTALFKNTNTKTKDNFGYSKRSTHKSGKVEEDVMGSIEVDYDGVVF
SIGTGFDADQRRDFWQNKESYIGKMVKFKYFEMGSKDCPRFPVFIGIRH
EEDR

The kit, composition, or RNA detection and/or quantification system may comprise one or more DNA ligase buffers. A “DNA ligase buffer” may be a concentrated buffer which can be added to a composition to provide suitable conditions for activity of the corresponding DNA ligase. Suitably, a DNA ligase buffer is 5× to 100× concentrated.

Suitably, the buffer is a Splint R ligase buffer. Suitably, a 1× Splint R ligase buffer comprises 1 μM-1.5 mM ATP or 10 μM 1 mM ATP and/or has a pH of 7.5-8.0, e.g. pH 7.5 or pH 7.6 at 25° C. Suitably, a 1× Splint R ligase buffer comprises 66 mM Tris-HCl, 10 mM MgCl₂, 1 mM ATP, 1 mM DTT, 7.5% PEG or 50 mM Tris-HCl, 10 mM MgCl₂, 1 mM ATP, 10 mM DTT.

Exonucleases

The kit, composition, or RNA detection and/or quantification system may comprise one or more exonucleases. An “exonuclease” is an enzyme which removes successive nucleotides from the end of a polynucleotide molecule. The exonuclease may be any exonuclease suitable for exonuclease digestion of non-circularised padlock probes. Suitable exonucleases will be well known to those of skill in the art. Suitable exonucleases include Exo I, Lambda Exo and T5 Exo.

Amplification, Detection, and Quantification Reagents

The kit, composition, or RNA detection and/or quantification system may comprise one or more DNA polymerases. A “DNA polymerase” is an enzyme that synthesizes DNA molecules from nucleoside triphosphates. During this process, DNA polymerase reads the existing DNA strand and creates a new strand. The DNA polymerase may be any suitable DNA polymerase. Suitable DNA polymerases will be well known to those of skill in the art. A suitable DNA polymerase is a Bst DNA polymerase Warmstart.

The kit, composition, or RNA detection and/or quantification system may comprise one or more amplification buffers. An “amplification buffer” may be a concentrated buffer which can be added to a composition to provide suitable conditions for activity of a DNA polymerase. Suitably, an amplification buffer is 5× to 100× concentrated. Suitable amplification buffers will be well known to those of skill in the art.

The kit, composition, or RNA detection and/or quantification system may comprise one or more deoxynucleoside triphosphate (dNTP) mixes. A “dNTP mix” may be a premixed aqueous solutions of dATP, dCTP, dGTP and dTTP suitable for activity of a DNA polymerase. Suitably, a dNTP mix comprises 10 mM of each of dATP, dCTP, dGTP and dTTP.

The kit, composition, or RNA detection and/or quantification system may comprise one or more DNA dyes. A “DNA dye” may be any dye which binds to double-stranded DNA, which increases the fluorescence quantum yield of the dye. An increase in DNA product during amplification therefore leads to an increase in fluorescence intensity. The DNA dye may be any suitable DNA dye. Suitable DNA dyes will be well known to those of skill in the art. A suitable DNA dye is SYBR Green I.

In some embodiments, the kit, composition, or RNA detection and/or quantification system comprises one or more DNA polymerases, one or more amplification buffers, one or more dNTP mixes, and one or more DNA dyes.

Reference Samples

The kit, composition, or RNA detection and/or quantification system may comprise one or more reference samples. For example, the kit, composition, or RNA detection and/or quantification system may comprise one or more reference samples comprising a known RNA at a known concentration. The reference samples may be suitable for calibration when quantifying an RNA of interest.

Reference samples may be prepared by any suitable method known to those of skill in the art. For example, a reference sample may be prepared by a method comprising: (i) obtaining a reference DNA template encoding an RNA; (ii) transcribing the reference DNA to obtain a transcribed RNA; and (iii) isolating and purifying the transcribed RNA.

Components for Detection and/or Quantification of Corresponding Genes

When the kit, composition, or RNA detection and/or quantification system comprises one or more padlock probes for detecting a gene which encodes an RNA of interest, the kit, composition, or RNA detection and/or quantification system may comprise any additional components necessary to detect and/or quantify the gene which encodes an RNA of interest.

The kit, composition, or RNA detection and/or quantification system may comprise one or more primers specific for one or more primer binding sites on the padlock probes for detecting and/or quantifying a gene which encodes an RNA of interest.

In some embodiments the kit, composition, or RNA detection and/or quantification system of the present invention comprises: (i) one or more padlock probes for detecting and/or quantifying a gene which encodes an RNA of interest comprising or consisting of from 5′ to 3′: a first terminal region; a first primer binding site; a second primer binding site; and a second terminal region; (ii) one or more first primers; and (iii) one or more second primers. Suitably, the first primer is a forward primer and the second primer is a reverse primer, or vice versa.

For example, the kit, composition, or RNA detection and/or quantification system of the present invention may comprise:
(a) a padlock probe consisting of the nucleotide sequence of SEQ ID NO: 71, a forward primer consisting of the nucleotide sequence of SEQ ID NO: 86, and a reverse primer consisting of the nucleotide sequence of SEQ ID NO: 87; and/or
(b) a padlock probe consisting of the nucleotide sequence of SEQ ID NO: 72, a forward primer consisting of the nucleotide sequence of SEQ ID NO: 88 and a reverse primer consisting of the nucleotide sequence of SEQ ID NO: 89.

Exemplary primers for a padlock probe consisting of SEQ ID NO: 71:
Forward primer
(SEQ ID NO: 86)
ACACTTCCAAACTCTCTCAATC
Reverse primer
(SEQ ID NO: 87)
CTGTCCAGGGATCTGCTCTT
Exemplary primers for a padlock probe consisting of SEQ ID NO: 72:
Forward primer
(SEQ ID NO: 88)
GAGTCCTGCTACAAGATTTCA
Reverse primer
(SEQ ID NO: 89)
TGGACAGGCAAGGAATACAGG

The kit, composition, or RNA detection and/or quantification system may comprise one or more capture probes specific for the one or more padlock probes for detecting and/or quantifying a gene which encodes an RNA of interest.

For example, the kit, composition, or RNA detection and/or quantification system of the present invention may comprise:
(a) a padlock probe consisting of the nucleotide sequence of SEQ ID NO: 71 and a capture probe consisting of the nucleotide sequence of SEQ ID NO: 90, and optionally a forward primer consisting of the nucleotide sequence of SEQ ID NO: 86, and a reverse primer consisting of the nucleotide sequence of SEQ ID NO: 87; and/or
(b) a padlock probe consisting of the nucleotide sequence of SEQ ID NO: 72 and a capture probe consisting of the nucleotide sequence of SEQ ID NO: 90, and optionally a forward primer consisting of the nucleotide sequence of SEQ ID NO: 88 and a reverse primer consisting of the nucleotide sequence of SEQ ID NO: 89.

Exemplary capture probe for a padlock probe consisting of SEQ ID NO: 71 or 72
(SEQ ID NO: 90):
Biot 5′ ACA-GTC-AGA-GGT-TCA-ATT-CCT-CTT-CT 3′ spacer C3

The kit, composition, or RNA detection and/or quantification system may comprise one or more DNA ligases which can efficiently ligate padlock probes when they are hybridised to DNA. Suitable DNA ligases will be well known to those of skill in the art. A suitable DNA ligase is a Taq DNA ligase.

Methods of Detecting and/or Quantifying RNA

The padlock probes of the present invention may be used to detect RNA, for example RNA comprising the target site. The padlock probes of the present invention may also be used to quantify said RNA.

In one aspect, the present invention provides for use of a padlock probe for detecting and/or quantifying a RNA. The RNA may be any RNA described herein. The padlock probe may be any padlock probe described herein.

In one aspect, the present invention provides a method of detecting and/or quantifying a RNA using one or more padlock probes. The RNA may be any RNA described herein. The padlock probe may be any padlock probe described herein.

The method may comprise:
(a) providing a sample comprising one or more RNAs;
(b) hybridising one or more padlock probes to the one or more RNAs to obtain one or more hybridised padlock probes;
(c) circularising the one or more hybridised padlock probes to obtain one or more circularised padlock probes;
(d) optionally purifying the one or more circularised padlock probes;
(e) amplifying the one or more circularised padlock probes to obtain amplified padlock probes;
(f) detecting the amplified padlock probes; and
(g) optionally quantifying the one or more RNAs.

Step (a): Providing a Sample Comprising One or More RNAs

Methods to obtain a sample comprising one or more RNAs will be well known to those of skill in the art. Suitably, RNA may be extracted from one or more tissues and subsequently purified and/or isolated to provide a purified sample comprising one or more RNAs.

The sample comprising one or more RNAs may be an RNA sample. As used herein, an “RNA sample” is a sample consisting essentially of RNA and an aqueous medium (e.g. water or a buffer). An RNA sample may consist of RNA and an aqueous medium (e.g. water or a buffer). An RNA sample may comprise essentially no DNA, i.e. an RNA sample may contain non-detectable amounts of DNA. Suitably, the sample is a purified and/or isolated RNA sample.

The sample comprising one or more RNAs may be obtained or obtainable from urine, muscle, blood and/or saliva. Suitably the sample is obtained or obtainable from urine. Urine is an attractive tissue for diagnostics since it is obtained in a fully non-invasive manner. Suitably the sample is obtained or obtainable from blood.

The method may further comprise a step of extracting, purifying and/or isolating the sample from tissue. As used herein, “extracting” may be a step of extracting nucleic acids from a tissue to obtain a crude sample comprising one or more RNAs. As used herein, “purifying” or “isolating” may be a step of separating the desired nucleic acids from a crude sample to provide an RNA sample. Any suitable method may be used and commercial kits for extracting, purifying and/or isolating RNA are available. Suitably, PAXgene blood RNA tubes may be used according to the commercial protocol. Suitably, the sample can be transported on dry ice and/or stored at −80° C.

The method may further comprise a step of modifying the RNA.

Suitably, if the RNA is tRNA, an oligonucleotide is ligated to the 3′ end of the tRNA. This ligation is possible only if the tRNA is not AA-charged, since otherwise the 3′ end of the tRNA is not free but bound to the amino acid.

The oligonucleotide is suitably a heteroduplex composed of an RNA oligonucleotide and a DNA oligonucleotide (i.e. an RNA/DNA heteroduplex) or a DNA oligonucleotide duplex. Suitably, the (hetero)duplex is formed from two complementary oligonucleotides and/or comprises a TGG-3′ overhang. Optionally, the oligonucleotide is an RNA/DNA heteroduplex comprising a TGG-3′ overhang of the DNA oligonucleotide. For example, the oligonucleotide may consist of: (i) a DNA oligonucleotide consisting of from 5′ to 3′: (N)ₓ-TGG, where x is 1 to 100, 5 to 50, or 10 to 30; and (ii) a RNA oligonucleotide comprising or consisting of the reverse complement of (N)ₓ. Using such a RNA/DNA heteroduplex is advantageous because it can lead to a ligation product that is entirely composed of RNA.

Conditions suitable for ligation of an oligonucleotide to the 3′ end of the tRNA will be well known to those of skill in the art. Suitably, if the oligonucleotide is a RNA/DNA heteroduplex comprising a TGG-3′ overhang of the DNA oligonucleotide, an RNA polymerase may be used (e.g. T4 RNA ligase 2).

Step (b): Hybridising One or More Padlock Probes to the One or More RNAs to Obtain One or More Hybridised Padlock Probes

Suitable methods for hybridisation will be well known to those of skill in the art. Suitably, the one or more padlock probes may be mixed with the one or more RNAs in a hybridisation buffer and incubated to obtain one or more RNA/padlock probe complexes. The padlock probes may be any padlock probe of the invention.

Any suitable amount of padlock probe and RNA may be used. Suitably, about 0.1 pmol of padlock probe and about 40-60 ng of RNA template can be mixed.

Any suitable hybridisation buffer may be used. The hybridisation buffer may be a 1×DNA ligase buffer, for example a 1× Splint R ligase buffer.

Any suitable incubation may be used. For example, a temperature ramp may be used in which the nucleic acids are heated to a high temperature to denature the nucleic acids, followed by a gradual decrease in temperature to enable specific hybridisation of the padlock probe to the target site. Suitably, a temperature ramp of between about 97° C. and about 37° C., with about a 5° C. decrement every 2 minutes may be used.

Optionally, the method may comprise a step of purifying the one or more RNA/padlock probe complexes prior to ligation of the padlock probe. Suitable methods for purifying the one or more RNA/circularised padlock probe complexes will be well known to those of skill in the art (e.g. the method described in Zhang, D. Y., et al., 1998. Gene, 211(2), pp. 277-285 or Smith, J. H. and Beals, T. P., 2013. PloS one, 8(5), p. e65053). For example, the one or more RNA/padlock probe complexes can be purified by bead separation, optionally magnetic bead separation e.g. by the method described below.

Step (c): Circularising the One or More Hybridised Padlock Probes to Obtain One or More Circularised Padlock Probes

Suitable methods for circularising the padlock probes will be well known to those of skill in the art. Suitably, the one or more hybridised padlock probes (i.e. RNA/padlock probe complexes, optionally purified) are mixed with one or more DNA ligases in a DNA ligase buffer and incubated to obtain one or more circularised padlock probes (i.e. RNA/circularised padlock probe complexes).

Any suitable DNA ligases and corresponding DNA ligase buffers may be used. The DNA ligase may be a Splint R ligase and the DNA ligase buffer may be 1× Splint R ligase buffer. Optionally, the DNA ligase buffer is the same as the hybridisation buffer.

Any suitable incubation may be used. Suitably, the hybridised probes may be incubated with the DNA ligase at about 37° C. for about 45 minutes or less.

Step (d): Purifying the One or More Circularised Padlock Probes

Suitable methods for purifying the one or more circularised padlock probes will be well known to those of skill in the art.

In some embodiments, the one or more circularised padlock probes are purified by bead separation, optionally magnetic bead separation. Suitable methods will be well known to those of skill in the art (e.g. the method described in Zhang, D. Y., et al., 1998. Gene, 211(2), pp. 277-285 or Smith, J. H. and Beals, T. P., 2013. PloS one, 8(5), p. e65053). Suitably the following steps may be used:
(i) the one or more RNA/circularised padlock probe complexes may be hybridised to one or more capture probes to obtain RNA/circularised padlock probes/capture probe complexes. The one or more capture probes may be any suitable capture probes, e.g. biotinylated capture probes, for example those describe herein;
(ii) the one or more RNA/circularised padlock probe/capture probe complexes may be mixed with beads and incubated to obtain beads loaded with the one or more circularised padlock probes. The beads may be any suitable beads specific for the one or more capture probes, e.g. Streptavidin-coated beads, for example those described herein;
(iii) the beads may be isolated by centrifugation and/or by magnetic separation, and optionally washed to obtain purified beads loaded with the one or more RNA/circularised padlock probe/capture probe complexes; and
(iv) the one or more RNA/circularised padlock probe/capture probe complexes may be eluted from the bead and isolated. Suitably, the complexes are eluted by thermal denaturation, e.g. at about 80° C. for about 5 minutes.

In some embodiments, non-circularised padlock probes and other nucleic acids (e.g. the RNA and capture probes) are digested. Suitably, the digestion may occur after bead separation. Suitable methods will be well known to those of skill in the art. Suitably, one or more exonucleases are mixed with the sample containing the circularised padlock probes (e.g. a sample containing eluted RNAs, circularised padlock probes, and capture probes) in a suitable buffer and incubated. Exonucleases will not digest the circularised padlock probes. The one or more exonuclease may be any suitable exonucleases, for example those described herein. Suitably, the buffer is a 1× amplification buffer. Suitably the incubation is for about 90 minutes at about 37° C. The one or more exonucleases should be inactivated prior to step (e), for example by incubation for about 20 minutes at about 80° C.

Step (e): Amplifying the One or More Circularised Padlock Probes to Obtain Amplified Padlock Probes

Suitable methods for amplifying the one or more circularised padlock probes will be well known to those of skill in the art. Suitably, the one or more circularised padlock probes are mixed with one or more primers, one or more DNA polymerases, and one or more dNTP mixes in an amplification buffer, and incubated. Any suitable primers, DNA polymerase, dNTP mixes and amplification buffers may be used, for example those described herein.

Suitably, the one or more padlock probes are amplified by rolling circle amplification (RCA), particularly when the padlock probe comprises one primer binding site. Suitable conditions will be well known to those of skill in the art and are described in Ali, M. M., et al., 2014. Chemical Society Reviews, 43(10), pp. 3324-3341.

The RCA may be exponential RCA. By employing multiple primers that hybridise with the same circle, multiple amplification events can be initiated producing multiple RCA products. Exponential RCA can be used to improve the sensitivity of detection. For example, the exponential RCA may be multi-primed RCA or hyper-branched RCA (HRCA).

Suitably, the one or more padlock probes are amplified by HRCA, particularly when the padlock probe comprises at least one forward primer binding site and at least one reverse primer binding site. In HRCA the RCA product produced using the forward primer is used as the template for further amplification with the reverse primer.

Suitable incubation conditions will be well known to those of skill in the art. For example, if Bst DNA polymerase Warmstart is used, the incubation may be at about 65° C. for about 1 hour.

Step (f): Detecting the Amplified Padlock Probes.

Suitable methods for detecting the amplified circularised padlock probes will be well known to those of skill in the art. Suitably, the amplified circularised padlock probe is mixed with a DNA dye and the presence of the amplified padlock probe is detected by an increase in fluorescence. Any suitable DNA may be used, for example those described herein.

Suitably, the DNA dye is added during amplification, such that the amplified padlock probe (e.g. the increase in fluorescence) can be detected in real-time.

Suitably, the amplified circularised padlock probes are detected using a label-free detection method e.g. using biosensors comprising field-effect transistors. For example, by a method described in US2006246443A1 or US2006011911A1.

Suitably, the amplified circularised padlock probes may be detected using a sensor consisting of a network of field-effect transistors (T1, T2 etc.), each of which has a source region (S), a drain region (D), and a gate region which forms an active zone. The method using said sensor may comprise:
a) bringing at least one active zone into contact with probe molecules fixed to said active zone,
b) bringing at least some of the probe molecules into contact with target biomolecules capable of interaction with said probe molecules, and performing a said interaction in a reaction buffer having a first salt concentration, and
c) measuring at least one point of the drain current/source-gate voltage/source-drain voltage characteristic of at least one transistor of said array to detect said specific interaction at least for a measurement point obtained in a measuring buffer having a second salt concentration that is lower than the first concentration for probe molecules having been subjected to said specific interaction.

Step (g): Quantifying the One or More RNAs

Suitable methods for quantifying the one or more RNAs will be well known to those of skill in the art. Suitably, the fluorescence intensity of the amplified padlock probes is compared to one or more reference samples containing a known concentration of RNA.

Suitably, the one or more RNAs are quantified by quantitative polymerase chain reaction (qPCR). Real-time detection of fluorescence intensity can be used quantitatively. For example, if the increase in fluorescence is detected in real-time, the fluorescence signal will increase with time, and finally saturate. The increase occurs more rapidly for higher RNA concentrations. The time at which the relative signal passes a predetermined threshold can therefore be used to quantify the RNA concentration. qPCR is described in Taylor, S. C., et al., 2019. Trends in biotechnology, 37(7), pp. 761-774.

Methods of Detecting and/or Quantifying RNA Mutations

The padlock probes of the present invention may be used to detect and/or quantify mutant RNA.

In one aspect, the present invention provides for use of a padlock probe for detecting and/or quantifying a mutant RNA.

In a related aspect, the present invention provides a method of detecting and/or quantifying a mutant RNA using one or more padlock probes.

The mutant RNA may be any mutant RNA described herein. The padlock probe may be any padlock probe described herein. Suitably, the padlock probe hybridises to a target region comprising the mutation. Suitably, one of the terminal regions hybridises to a target region comprising the mutation.

The method may comprise:
(a) providing a sample comprising one or more mutant RNAs;
(b) hybridising one or more padlock probes to the one or more mutant RNAs to obtain one or more hybridised padlock probes;
(c) circularising the one or more hybridised padlock probes to obtain one or more circularised padlock probes;
(d) optionally purifying the one or more circularised padlock probes;
(e) amplifying the one or more circularised padlock probes to obtain amplified padlock probes;
(f) detecting the amplified padlock probes; and
(g) optionally quantifying the one or more mutant RNAs.

Suitably, step (g) may be used to determine the concentration of mutant RNAs in the sample (cₘᵤₜ).

Suitably, the sample further comprises one or more wild-type RNAs (i.e. RNAs corresponding to the one or more mutant RNAs but not comprising the mutation). Suitably, the method may further comprise:
(b′) hybridising one or more padlock probes to the one or more wild-type RNAs to obtain one or more hybridised padlock probes;
(c′) circularising the one or more hybridised padlock probes to obtain one or more circularised padlock probes;
(d′) optionally purifying the one or more circularised padlock probes;
(e′) amplifying the one or more circularised padlock probes to obtain amplified padlock probes;
(f′) detecting the amplified padlock probes; and
(g′) optionally quantifying the one or more wild-type RNAs.

The padlock probe may be any padlock probe described herein. Suitably, the padlock probe hybridises to a target region comprising the mutation site. Suitably, one of the terminal regions hybridises to a target region comprising the mutation site. Suitably, the padlock probe terminal regions are identical to those used in step (b), except the nucleotide corresponding to the mutation site, which is complementary to the wild-type sequence.

Suitably, step (g′) may be used to determine the concentration of wild-type RNAs in the sample (c_{wt}).

Suitably, step (g) and step (g′) may be used to calculate the RNA mutation load, cₘᵤₜ/(c_{wt}+cₘᵤₜ). The RNA mutation load corresponds to the percentage of RNA molecules in the sample which were mutant RNAs, i.e. comprised the mutation. This may also be referred to as the RNA heteroplasmy level, or RNA mutation frequency.

Alternatively, the method may further comprise:
(b′) hybridising one or more padlock probes to the one or more mutant RNAs and to one or more wild-type RNAs to obtain one or more hybridised padlock probes;
(c′) circularising the one or more hybridised padlock probes to obtain one or more circularised padlock probes;
(d′) optionally purifying the one or more circularised padlock probes;
(e′) amplifying the one or more circularised padlock probes to obtain amplified padlock probes;
(f′) detecting the amplified padlock probes; and
(g′) optionally quantifying the one or more mutant RNAs and one or more wild-type RNAs.

The padlock probe may be any padlock probe described herein. Suitably, the padlock probe hybridises to a target region which does not comprise the mutation site. Suitably, both terminal regions hybridise to target regions which do not comprise the mutation site.

Suitably, step (g′) may be used to determine the concentration of total RNA (i.e. mutant and wild-type) in the sample (cₜₒₜₐₗ=c_{wt}+cₘᵤₜ).

Suitably, step (g) and step (g′) may be used to calculate the RNA mutation load, cₘᵤₜ/cₜₒₜₐₗ.

Suitably, the sample further comprises one or more reference RNAs. The reference RNAs may be any suitable reference RNA, for example those described herein. Suitably, the method may further comprise:
(b″) hybridising one or more padlock probes to the one or more reference RNAs to obtain one or more hybridised padlock probes;
(c″) circularising the one or more hybridised padlock probes to obtain one or more circularised padlock probes;
(d″) optionally purifying the one or more circularised padlock probes;
(e″) amplifying the one or more circularised padlock probes to obtain amplified padlock probes;
(f″) detecting the amplified padlock probes; and
(g″) optionally quantifying the one or more reference RNAs.

The padlock probe may be any padlock probe described herein.

Suitably, step (g″) may be used to determine the concentration of reference RNAs in the sample (c_{ref}). Suitably, this may be used to calculate the relative quantity of wild-type RNA molecules, c_{wt}/c_{ref} and/or the relative quantity of mutant molecules, cₘᵤₜ/cr_{ef}.

Suitably, the method may further comprise detecting and/or quantifying the corresponding genes encoding the RNA. Suitably, the method may further comprise:
(a′″) providing a sample comprising one or more polynucleotides encoding the mutant RNA;
(b′″) hybridising one or more padlock probes to the one or more polynucleotides encoding the mutant RNA to obtain one or more hybridised padlock probes;
(c′″) circularising the one or more hybridised padlock probes to obtain one or more circularised padlock probes;
(d′″) optionally purifying the one or more circularised padlock probes;
(e′″) amplifying the one or more circularised padlock probes to obtain amplified padlock probes;
(f′″) detecting the amplified padlock probes; and
(g′″) optionally quantifying the one or more polynucleotides encoding the mutant RNA.

Methods to obtain a sample comprising one or more polynucleotides encoding the mutant RNA will be well known to those of skill in the art. Suitably, polynucleotides may be extracted from one or more tissues and subsequently purified and/or isolated to provide a purified sample comprising one or more polynucleotides encoding the mutant RNA.

The sample comprising one or more polynucleotides encoding the mutant RNA may be a DNA sample. As used herein, a “DNA sample” is a sample consisting essentially of DNA and an aqueous medium (e.g. water or a buffer). A DNA sample may consist of DNA and an aqueous medium (e.g. water or a buffer). A DNA sample may comprise essentially no RNA, i.e. a DNA sample may contain non-detectable amounts of RNA. Suitably, the sample is a purified and/or isolated DNA sample.

The sample comprising one or more polynucleotides encoding the mutant RNA may be obtained or obtainable from urine, muscle, blood and/or saliva. The method may further comprise a step of extracting, purifying and/or isolating the sample from tissue. Any suitable method may be used and commercial kits for extracting, purifying and/or isolating DNA are available. Suitably, PAXgene blood DNA tubes may be used according to the commercial protocol. Suitably, the sample can be transported on dry ice and/or stored at −80° C.

The padlock probe may be any padlock probe described herein for detecting and/or quantifying the gene which encodes the RNA. Suitably, the padlock probe hybridises to a target region comprising the mutation. Suitably, one of the terminal regions hybridises to a target region comprising the mutation.

Suitably, step (g′″) may be used to determine the concentration of mutant RNA genes in the sample (c′ₘᵤₜ).

Suitably, the sample comprises one or more polynucleotides encoding the wild-type RNA.

Suitably, the method may further comprise:
(a″″) providing a sample comprising one or more polynucleotides encoding the wild-type RNA;
(b″″) hybridising one or more padlock probes to the one or more polynucleotides encoding the wild-type RNA to obtain one or more hybridised padlock probes;
(c″″) circularising the one or more hybridised padlock probes to obtain one or more circularised padlock probes;
(d″″) optionally purifying the one or more circularised padlock probes;
(e″″) amplifying the one or more circularised padlock probes to obtain amplified padlock probes;
(f″″) detecting the amplified padlock probes; and
(g″″) optionally quantifying the one or more polynucleotides encoding the mutant RNA.

The padlock probe may be any padlock probe described herein for detecting and/or quantifying the gene which encodes the RNA. Suitably, the padlock probe hybridises to a target region comprising the mutation site. Suitably, one of the terminal regions hybridises to a target region comprising the mutation site. Suitably, the padlock probe terminal regions are identical to those used in step (b), except the nucleotide corresponding to the mutation site, which is complementary to the wild-type sequence.

Suitably, step (g″″) may be used to determine the concentration of wild-type RNA genes in the sample (c′_{wt}).

Suitably, step (g′″) and step (g″″) may be used to calculate the DNA mutation load, c′ₘᵤₜ/(c′_{wt}+c′ₘᵤₜ). The DNA mutation load corresponds to the percentage of DNA molecules in the sample which encoded mutant RNAs. This may also be referred to as the DNA heteroplasmy level, or DNA mutation frequency.

Methods of Detecting Aberrant RNA Post-Transcriptional Modifications

The padlock probes of the present invention may be used to detect and/or quantify aberrantly-modified RNA (i.e. RNA with aberrant post-transcriptional modifications).

In one aspect, the present invention provides for use of a padlock probe for detecting and/or quantifying an aberrantly-modified RNA.

In a related aspect, the present invention provides a method of detecting and/or quantifying an aberrantly-modified RNA using one or more padlock probes.

The aberrantly-modified RNA may be any aberrantly-modified RNA described herein. The padlock probe may be any padlock probe described herein. Suitably, the padlock probe hybridises to a target region comprising the aberrant post-transcriptional modification.

Suitably, one of the terminal regions hybridises to a target region comprising the aberrant post-transcriptional modification.

The method may comprise:
(a) providing a sample comprising one or more aberrantly-modified RNAs;
(b) hybridising one or more padlock probes to the one or more aberrantly-modified RNAs to obtain one or more hybridised padlock probes;
(c) circularising the one or more hybridised padlock probes to obtain one or more circularised padlock probes;
(d) optionally purifying the one or more circularised padlock probes;
(e) amplifying the one or more circularised padlock probes to obtain amplified padlock probes;
(f) detecting the amplified padlock probes; and
(g) optionally quantifying the one or more aberrantly-modified RNAs.

Suitably, step (g) may be used to determine the concentration of aberrantly-modified RNAs in the sample (cₘᵤₜ).

Suitably, the sample further comprises one or more wild-type RNAs (i.e. RNAs corresponding to the one or more aberrantly-modified RNAs but not comprising the aberrantly-modified). Suitably, the method may further comprise:
(b′) hybridising one or more padlock probes to the one or more aberrantly-modified and to one or more wild-type RNAs to obtain one or more hybridised padlock probes;
(c′) circularising the one or more hybridised padlock probes to obtain one or more circularised padlock probes;
(d′) optionally purifying the one or more circularised padlock probes;
(e′) amplifying the one or more circularised padlock probes to obtain amplified padlock probes;
(f′) detecting the amplified padlock probes; and
(g′) optionally quantifying the one or more aberrantly-modified and one or more wild-type RNAs.

The padlock probe may be any padlock probe described herein. Suitably, the padlock probe hybridises to a target region which does not comprise the aberrant modification site.

Suitably, both terminal regions hybridise to target regions which do not comprise the aberrant modification site.

Suitably, step (g′) may be used to determine the concentration of total RNA in the sample (cₜₒₜₐₗ).

Suitably, step (g) and step (g′) may be used to calculate the RNA aberrant-modification load, cₘᵤₜ/cₜₒₜₐₗ. The RNA aberrant-modification load corresponds to the percentage of RNA molecules in the sample which were aberrantly modified.

Suitably, the sample further comprises one or more reference RNAs. The reference RNAs may be any suitable reference RNA, for example those described herein. Suitably, the method may further comprise:
(b″) hybridising one or more padlock probes to the one or more reference RNAs to obtain one or more hybridised padlock probes;
(c″) circularising the one or more hybridised padlock probes to obtain one or more circularised padlock probes;
(d″) optionally purifying the one or more circularised padlock probes;
(e″) amplifying the one or more circularised padlock probes to obtain amplified padlock probes;
(f″) detecting the amplified padlock probes; and
(g″) optionally quantifying the one or more reference RNAs.

The padlock probe may be any padlock probe described herein.

Suitably, step (g″) may be used to determine the concentration of reference RNAs in the sample (c_{ref}). Suitably, this may be used to calculate the relative quantity of aberrantly-modified molecules, cₘᵤₜ/c_{ref}.

Methods of Detecting tRNA Amino Acid-Charging

The padlock probes of the present invention may be used to detect and/or quantify tRNA amino acid-charging.

In one aspect, the present invention provides for use of a padlock probe for detecting and/or quantifying tRNA amino acid-charging.

In a related aspect, the present invention provides a method of detecting and/or quantifying tRNA amino acid-charging using one or more padlock probes.

The tRNA may be any tRNA described herein comprising a 3′ ligated oligonucleotide. To detect and/or quantify tRNAs which are not amino acid-charged, an oligonucleotide can be ligated to the 3′ end of the tRNA.

The oligonucleotide is suitably a heteroduplex composed of an RNA oligonucleotide and a DNA oligonucleotide (i.e. an RNA/DNA heteroduplex) or a DNA oligonucleotide duplex. Suitably, the (hetero)duplex is formed from two complementary oligonucleotides and/or comprises a TGG-3′ overhang. Optionally, the oligonucleotide is an RNA/DNA heteroduplex comprising a TGG-3′ overhang of the DNA oligonucleotide. For example, the oligonucleotide may consist of: (i) a DNA oligonucleotide consisting of from 5′ to 3′: (N)ₓ-TGG, where x is 1 to 100, 5 to 50, or 10 to 30; and (ii) a RNA oligonucleotide comprising or consisting of the reverse complement of (N)ₓ. Using such a RNA/DNA heteroduplex is advantageous because it can lead to a ligation product that is entirely composed of RNA.

The oligonucleotide can be ligated using any suitable method. For example, if the oligonucleotide is a RNA/DNA heteroduplex comprising a TGG-3′ overhang of the DNA oligonucleotide, an RNA polymerase may be used (e.g. T4 RNA ligase 2). The padlock probe hybridises to a target region comprising at least part of the nucleotide sequence of the 3′ ligated oligonucleotide. Suitably, a terminal region is complementary to at least part of the nucleotide sequence of the 3′ ligated oligonucleotide.

The method may comprise:
(a) providing a sample comprising one or more tRNAs comprising a 3′ ligated oligonucleotide;
(b) hybridising one or more padlock probes to the one or more mutant tRNAs to obtain one or more hybridised padlock probes;
(c) circularising the one or more hybridised padlock probes to obtain one or more circularised padlock probes;
(d) optionally purifying the one or more circularised padlock probes;
(e) amplifying the one or more circularised padlock probes to obtain amplified padlock probes;
(f) detecting the amplified padlock probes; and
(g) optionally quantifying the one or more tRNAs.

The method of may further comprise a step of ligating a oligonucleotide the 3′ end of one or more tRNAs. Suitably, step (a) may comprise:
(a1) providing a sample comprising one or more tRNAs which are not amino acid-charged; and
(a2) ligating one or more oligonucleotides to the 3′ end of the one or more tRNAs to provide a sample comprising one or more tRNAs comprising a 3′ ligated oligonucleotide.

This ligation is possible only if the tRNA is not AA-charged, since otherwise the 3′ end of the tRNA is not free but bound to the amino acid.

Suitably, step (g) may be used to determine the concentration of tRNAs in the sample which are not charged with an amino acid (cₘᵤₜ).

Suitably, the sample in step (a) further comprises one or more tRNAs which do not comprise a 3′ ligated oligonucleotide. For example, the sample in step (a1) further comprises one or more tRNAs which are amino-acid charged and thus the oligonucleotide is not ligated to the 3′ end of the tRNA. Suitably, the method may further comprise:
(b′) hybridising one or more padlock probes to the one or more tRNAs comprising a 3′ ligated oligonucleotide and to one or more tRNAs which do not comprise a 3′ ligated oligonucleotide to obtain one or more hybridised padlock probes;
(c′) circularising the one or more hybridised padlock probes to obtain one or more circularised padlock probes;
(d′) optionally purifying the one or more circularised padlock probes;
(e′) amplifying the one or more circularised padlock probes to obtain amplified padlock probes;
(f′) detecting the amplified padlock probes; and
(g′) optionally quantifying the one or more tRNAs comprising a 3′ ligated oligonucleotide and the one or more tRNAs which do not comprise a 3′ ligated oligonucleotide.

The padlock probe may be any padlock probe described herein. Suitably, the padlock probe hybridises to a target region which does not comprise the 3′ ligated oligonucleotide. Suitably, both terminal regions hybridise to target regions which do not comprise the 3′ ligated oligonucleotide.

Suitably, step (g′) may be used to determine the concentration of total tRNA in the sample (cₜₒₜₐₗ).

Suitably, step (g) and step (g′) may be used to calculate the tRNA amino acid charging percentage, cₘᵤₜ/cₜₒₜₐₗ. The tRNA amino acid charging percentage corresponds to the percentage of tRNA molecules in the sample which were not charged with an amino acids.

Suitably, the sample further comprises one or more reference tRNAs. The reference tRNAs may be any suitable reference tRNA, for example those described herein. Suitably, the method may further comprise:
(b″) hybridising one or more padlock probes to the one or more reference tRNAs to obtain one or more hybridised padlock probes;
(c″) circularising the one or more hybridised padlock probes to obtain one or more circularised padlock probes;
(d″) optionally purifying the one or more circularised padlock probes;
(e″) amplifying the one or more circularised padlock probes to obtain amplified padlock probes;
(f″) detecting the amplified padlock probes; and
(g″) optionally quantifying the one or more reference tRNAs.

The padlock probe may be any padlock probe described herein.

Suitably, step (g″) may be used to determine the concentration of reference tRNAs in the sample (c_{ref}). Suitably, this may be used to calculate the relative quantity of tRNA molecules in the sample which were not charged with an amino acids, cₘᵤₜ/c_{ref}.

Currently, a technique for identifying and quantifying tRNA amino-acid charging does not exist. The padlock probe or method of the invention may be used to determine the tRNAome of an organism or a type of cell (e.g. an expression host). Consequently, tRNAomes can be used to adapt the codons used when expressing an exogenous protein and improve the expression of the exogenous protein. The padlock probe or method of the invention therefore may be used to codon-optimise a nucleotide encoding a protein of interest and/or optimise an in vitro translation system.

Multiplexed Detection

A plurality of padlock probes of the present invention may be used to detect and/or quantify a plurality of RNAs. Suitably, each padlock probe comprises a unique tag or barcode sequence for specific detection and/or quantification. Suitably, each padlock probe is specific for a unique target sequence.

The unique target sequences may be in the same RNA. For example, the method may comprise:
(a) providing a sample comprising RNA;
(b) hybridising a first padlock probe to the RNA to obtain a first hybridised padlock probe and hybridising a second padlock probe to the RNA to obtain a second hybridised padlock probe;
(c) circularising the first and second hybridised padlock probes to obtain circularised padlock probes;
(d) optionally purifying the circularised padlock probes;
(e) amplifying the circularised padlock probes to obtain amplified padlock probes;
(f) detecting the amplified padlock probes; and
(g) optionally quantifying the RNA.

The unique target sequences may be on different RNAs. For example, the method may comprise:
(a) providing a sample comprising a first RNA and a second RNA;
(b) hybridising a first padlock probe to the first RNA to obtain a first hybridised padlock probe and hybridising a second padlock probe to the second RNA to obtain a second hybridised padlock probe;
(c) circularising the first and second hybridised padlock probes to obtain circularised padlock probes;
(d) optionally purifying the circularised padlock probes;
(e) amplifying the circularised padlock probes to obtain amplified padlock probes;
(f) detecting the amplified padlock probes; and
(g) optionally quantifying the first RNA and the second RNA.

Steps (e)-(g) may be carried out as described in US2012264630A1, e.g. by amplifying the circularised padlock probes using one or more primers according to US2012264630A1 (as described above).

Methods of Diagnosing Disease

The detection and/or quantification of RNA may be used to detect, diagnose and/or assess the clinical severity of a RNA-associated disease in a subject. Suitably, the subject is a human subject.

Accordingly, in one aspect the present invention provides for use of a padlock probe, suitably a padlock probe of the invention, in detecting, diagnosing and/or assessing the clinical severity of a RNA-associated disease.

RNA-Associated Disease

In higher eukaryotes, many different RNAs are encoded in and transcribed from genomic DNAs. Transcribed RNA molecules undergo multiple post-transcriptional processes such as splicing, editing, modification, translation, and degradation. A defect, mis-regulation, or malfunction of these processes often results in diseases in humans, and recently they have been referred to as “RNA diseases” or “RNA-associated diseases” (Kataoka, N., Mayeda, A. and Ohno, K., 2019. Frontiers in molecular biosciences, 6, p. 53).

Pre-mRNA splicing is one of the major processes for post-transcriptional regulatory steps in eukaryotes. Defects in this step result in many diseases, such as neuromuscular diseases and cancers. Long non-coding RNAs (lncRNAs) also play important roles in gene expression in cells. Some of them can be good markers for cancer progression. (Kataoka, N., Mayeda, A. and Ohno, K., 2019. Frontiers in molecular biosciences, 6, p. 53).

The padlock probe of the present invention can be used to detect and/or quantify mutant or aberrantly-modified RNA and therefore have utility in detecting, diagnosing and/or assessing the clinical severity of RNA-associated diseases.

A summary of representative disease-causing RNA mutations is provided by Cooper, T. A., Wan, L. and Dreyfuss, G., 2009. Cell, 136(4), pp. 777-793. Diseases associated with RNA mutations are shown in Table 8 below. Suitably, the RNA-associated disease may be selected from one or more of the diseases listed in Table 8.

TABLE 8
Exemplary RNA-associated diseases

| DISEASE | GENE/MUTATION |
|---|---|
| Prader Willi syndrome | SNORD116 |
| Spinal muscular atrophy (SMA) | SMN2 |
| Dyskeratosis congenita (X-linked) | DKC1 |
| Dyskeratosis congenita (autosomal dominant) | TERC |
| Dyskeratosis congenita (autosomal dominant) | TERT |
| Diamond-Blackfan anemia | RPS19, RPS24 |
| Shwachman-Diamond syndrome | SBDS |
| Treacher-Collins syndrome | TCOF1 |
| Prostate cancer | SNHG5 |
| Myotonic dystrophy, type 1 (DM1) | DMPK (RNA gain-of-function) |
| Myotonic dystrophy type 2 (DM2) | ZNF9 (RNA gain-of-function) |
| Spinocerebellar ataxia 8 (SCA8) | ATXN8/ATXN8OS (RNA |
| | gain-of-function) |
| Huntington's disease-like 2 (HDL2) | JPH3 (RNA gain-of-function) |
| Fragile X-associated tremor ataxia syndrome (FXTAS) | FMR1 (RNA gain-of-function) |
| Fragile X syndrome | FMR1 |
| X-linked mental retardation | UPF3B |
| Oculopharyngeal muscular dystrophy (OPMD) | PABPN1 |
| Human pigmentary genodermatosis | DSRAD |
| Retinitis pigmentosa | PRPF31 |
| Retinitis pigmentosa | PRPF8 |
| Retinitis pigmentosa | HPRP3 |
| Retinitis pigmentosa | PAP1 |
| Cartilage-hair hypoplasia (recessive) | RMRP |
| Autism | 7q22-q33 locus breakpoint |
| Beckwith-Wiedemann syndrome (BWS) | H19 |
| Charcot-Marie-Tooth (CMT) Disease | GRS |
| Charcot-Marie-Tooth (CMT) Disease | YRS |
| Amyotrophic lateral sclerosis (ALS) | TARDBP |
| Leukoencephalopathy with vanishing white matter | EIF2B1 |
| Wolcott-Rallison syndrome | EIF2AK3 |
| Mitochondrial myopathy and sideroblastic anemia (MLASA) | PUS1 |
| Encephalomyopathy and hypertrophic cardiomyopathy | TSFM |
| Hereditary spastic paraplegia | SPG7 |
| Leukoencephalopathy | DARS2 |
| Susceptibility to diabetes mellitus | LARS2 |
| Deafness | MTRNR1 |
| MELAS syndrome, deafness | MTRNR2 |
| SFRS1-associated cancer | SFRS1 |
| RBM5-assocated cancer | RBM5 |
| Multiple mt-tRNA associated diseases | mitochondrial tRNA mutations |
| miR-17-92 cluster-associated cancer | miR-17-92 cluster |
| miR-372/miR-373-associated cancer | miR-372/miR-373 |

tRNA-Associated Disease

The detection and/or quantification of RNA may be used to detect, diagnose and/or assess the clinical severity of a tRNA-associated disease in a subject. Suitably, the subject is a human subject.

Accordingly, in one aspect the present invention provides for use of a padlock probe, suitably a padlock probe of the invention, in detecting, diagnosing and/or assessing the clinical severity of a tRNA-associated disease.

Pathological mutations in tRNA genes and tRNA processing enzymes are numerous and result in very complicated clinical phenotypes. Multiple mutations in individual tRNA genes have been associated with multiple diseases, and individual diseases have been found to be caused by mutations in one of several tRNAs (Abbott, J. A., Francklyn, C. S. and Robey-Bond, S. M., 2014. Frontiers in genetics, 5, p. 158).

Suitably, a tRNA-associated disease is caused by a mutation in a gene encoding a tRNA and/or a mutation in a gene encoding a tRNA processing or modifying enzyme. Genetic disorders in which tRNA alterations are thought to play a direct part can be classified into two groups of pathogenic mutations: within the tRNAs or in the tRNA processing and modifying enzymes. Additionally, many diseases that do not have a direct mutational link to tRNAs and their associated enzymes nevertheless display alterations in tRNA pools, albeit usually as a secondary effect of the altered disease biology (S. Kirchner, Z. Ignatova, Nat. Rev. Genet. 16, 98-112 (2015)).

Suitably, a tRNA-associated disease is caused by a mutation in a gene encoding a tRNA. For all the identified cases in which human disease are directly linked to mutations in tRNAs, these mutations occur in mt-tRNAs (S. Kirchner, Z. Ignatova, Nat. Rev. Genet. 16, 98-112 (2015)).

The padlock probe of the invention can be used to assess the heteroplasmy level in one or more different tissue. Suitably, the tRNA-associated disease is heteroplasmic. The disease phenotypes of many of tRNA-based pathologies are potentiated in a tissue-specific manner (S. Kirchner, Z. Ignatova, Nat. Rev. Genet. 16, 98-112 (2015)). The mutation load (also called heteroplasmy level, or mutation frequency) can be measured on different tissues, the most studied ones being urine, muscle and blood. Mutation loads observed in the different tissues are correlated, but not identical (A. Lombes et al, Biochemie 100, 171-176 (2014); J. P. Grady et al, EMBO Mol Med. 10, e8262 (2018)).

The data on the relation between the mutation load measured on mtDNA and the clinical severity are under debate (P. F. Chinnery et al, Brain 120, 1713-1721 (1997) and S. J. Pickett et al, Ann Clin Transl Neurol. 5, 333-45 (2018)). The heteroplasmy level can vary considerably from one individual to another. There is a relation between mutation load and clinical severity, which seems to be stronger if the mutation load is measured in blood, as compared to urine or muscle tissues.

Suitably, the tRNA-associated disease is a mt-tRNA-associated disease. Diseases associated with mt-tRNA mutations are catalogued in variety of databases, such as MITOMAP and Online Mendelian Inheritance in Man (OMIM).

Suitably, the mt-tRNA disease is selected from one or more of: a mitochondrial encephalomyopathy, a mitochondrial cardiomyopathy, mitochondrial myopathy deafness/sensorineural hearing loss, or diabetes mellitus. Suitably, the mt-tRNA disease is a mitochondrial encephalomyopathy.

Mitochondrial encephalomyopathies are a group of clinically, genetically, and biochemically heterogeneous dis-orders caused by a defect in the oxidative phosphorylation pathway of the respiratory chain. Examples include MELAS syndrome and MERRF syndrome (DiMauro, S. and Hirano, M., 2005. Neuromuscular disorders, 15(4), pp. 276-286.).

Mitochondrial cardiomyopathies are myocardial disorders characterized by abnormal myocardial structure and/or function secondary to genetic defects resulting in the impairment of the mitochondrial respiratory chain, in the absence of concomitant coronary artery disease, hypertension, valvular disease, and congenital heart disease (El-Hattab, A. W. and Scaglia, F., 2016. Frontiers in cardiovascular Medicine, 3, p. 25).

Mitochondrial myopathies are clinically heterogeneous disorders that can affect multiple systems besides skeletal muscle and are usually defined by morphological abnormalities of muscle mitochondria (DiMauro, S., et al., 1985. Annals of Neurology: Official Journal of the American Neurological Association and the Child Neurology Society, 17(6), pp. 521-538).

Deafness is a degree of hearing loss such that a person is unable to understand speech, even in the presence of amplification. Sensorineural hearing loss is a type of hearing loss in which the root cause lies in the inner ear or sensory organ (cochlea and associated structures) or the vestibulocochlear nerve (cranial nerve VIII).

Diabetes mellitus (DM), commonly known as diabetes, is a group of metabolic disorders characterized by a high blood sugar level over a prolonged period of time.

Suitably, the mt-tRNA disease is selected from one or more of: Alzheimer's Disease and Parkinson Disease (ADPD); Ataxia, Myopathy, and Deafness (AMDF); Chronic Progressive Ophthalmoplegia (CPEO); Deafness/Sensorineural Hearing Loss (DEAF/SNHL); Dementia and Chorea (DEMCHO); Diabetes Mellitus (DM); Diabetes Mellitus and Deafness (DMDF); exercise intolerance (EXIT); Focal Segmental Glomerulosclerosis (FSGS); Gastrointestinal Reflux (GER); Leber Hereditary Optic Neuropathy (LHON); Leigh Syndrome (LS); Myoclonic Epilepsy and Ragged Red Fiber disease (MERRF); Mitochondrial Encephalomyopathy, Lactic acidosis and Stroke-like episodes (MELAS); Maternally Inherited Cardiomyopathy (MICM); Maternally Inherited Diabetes and Deafness (MIDD); Maternally Inherited Leigh Syndrome (MILS); mitochondrial myopathy (MM); Mitochondrial Myopathy and Cardiomyopathy (MMC); Mitochondrial NeuroGastrolntestinal Encephalopathy (MNGIE); Progressive Encephalomyopathy (PEM); Retinitis Pigmentosa (RP); Sudden Infant Death Syndrome (SIDS); Sensorineural Hearing Loss (SNHL).

Suitably, the mt-tRNA disease is MERRF or MELAS.

MELAS Syndrome

The detection and/or quantification of RNA may be used to detect, diagnose and/or assess the clinical severity of MELAS syndrome in a subject. Suitably, the subject is a human subject.

Accordingly, in one aspect the present invention provides for use of a padlock probe, suitably a padlock probe of the invention, in detecting, diagnosing and/or assessing the clinical severity of MELAS syndrome.

MELAS syndrome (Mitochondrial Encephalopathy, Lactic Acidosis and Stroke-like episodes) is a mitochondrial cytopathy associated with mutations of mitochondrial DNA.

MELAS syndrome affects many of the body's systems, particularly the brain and nervous system (encephalo-) and muscles (myopathy). The signs and symptoms of this disorder most often appear in childhood following a period of normal development, although they can begin at any age. Early symptoms may include muscle weakness and pain, recurrent headaches, loss of appetite, vomiting, and seizures. Most affected individuals experience stroke-like episodes beginning before age 40. These episodes often involve temporary muscle weakness on one side of the body (hemiparesis), altered consciousness, vision abnormalities, seizures, and severe headaches resembling migraines. Repeated stroke-like episodes can progressively damage the brain, leading to vision loss, problems with movement, and a loss of intellectual function (dementia).

Most people with MELAS have a buildup of lactic acid in their bodies, a condition called lactic acidosis. Increased acidity in the blood can lead to vomiting, abdominal pain, extreme tiredness (fatigue), muscle weakness, and difficulty breathing. Less commonly, people with MELAS may experience involuntary muscle spasms (myoclonus), impaired muscle coordination (ataxia), hearing loss, heart and kidney problems, diabetes, and hormonal imbalances.

MERRF Syndrome

The detection and/or quantification of RNA may be used to detect, diagnose and/or assess the clinical severity of MERRF syndrome in a subject. Suitably, the subject is a human subject.

Accordingly, in one aspect the present invention provides for use of a padlock probe, suitably a padlock probe of the invention, in detecting, diagnosing and/or assessing the clinical severity of MERRF syndrome.

MERRF syndrome (Myoclonic Epilepsy with Ragged Red Fibers) is a mitochondrial encephalomyopathy characterized by myoclonic seizures. The clinical diagnosis of MERRF is based on the following four “canonic” features: myoclonus, generalized epilepsy, ataxia, and ragged red fibers (RRF) in the muscle biopsy.

MERRF is characterized by muscle twitches (myoclonus), weakness (myopathy), and progressive stiffness (spasticity). When the muscle cells of affected individuals are stained and viewed under a microscope, these cells usually appear abnormal. These abnormal muscle cells are called ragged-red fibers. Other features of MERRF include recurrent seizures (epilepsy), difficulty coordinating movements (ataxia), a loss of sensation in the extremities (peripheral neuropathy), and slow deterioration of intellectual function (dementia). People with this condition may also develop hearing loss or optic atrophy, which is the degeneration (atrophy) of nerve cells that carry visual information from the eyes to the brain. Affected individuals sometimes have short stature and a form of heart disease known as cardiomyopathy. Less commonly, people with MERRF develop fatty tumors, called lipomas, just under the surface of the skin.

Due to the multiple symptoms presented by the individual, the severity of MERRF syndrome is currently very difficult to evaluate.

Methods of Detecting, Diagnosing and/or Assessing the Clinical Severity of a RNA-Associated Disease

A method of detecting and/or quantifying a RNA, as described herein may be used to detect, diagnose and/or assess the clinical severity of a RNA-associated disease. For example, the methods of detecting and/or quantifying a RNA may comprise a further step (h), of detecting, diagnosing and/or assessing the clinical severity of a RNA-associated disease based on the detection and or quantification of RNA in the sample.

A method of detecting, diagnosing and/or assessing the clinical severity of a RNA-associated disease may comprise detecting and/or quantifying mutant or aberrantly-modified RNAs in a sample. The RNA may be any RNA described herein, particularly a tRNA or a mt-tRNA. The sample may be any sample described herein, particularly a sample obtained or obtainable from urine, muscle, blood and/or saliva.

Suitably, the method of detecting, diagnosing and/or assessing the clinical severity of a RNA-associated disease may comprise:
(a) determining the concentration of wild-type RNAs (c_{wt}) in a sample;
(b) determining the concentration of mutant or aberrantly-modified RNAs (cₘᵤₜ) in the sample; and
(c) calculating the percentage RNA mutation load or aberrant-modification load in the sample, cₘᵤₜ/(c_{wt}+cₘᵤₜ)

Suitably, c_{wt} and cₘᵤₜ are determined by a method described herein.

The RNA mutation load or aberrant-modification load may be used to detect, diagnose and/or assess the clinical severity of a RNA-associated disease. A higher RNA mutation load mutation load may be associated with increased clinical severity of a RNA-associated disease.

In some embodiments, the method of detecting, diagnosing and/or assessing the clinical severity of a RNA-associated disease may further comprise:
(d) determining the concentration of a reference RNA (c_{ref}) in the sample; and
(e) calculating the relative quantity of wild-type RNA molecules in the sample, c_{wt}/c_{ref} and/or calculating the relative quantity of mutant or aberrantly-modified RNA molecules in the sample, cₘᵤₜ/c_{ref}.

Suitably, the reference RNA is a nuclear tRNA, optionally a nuclear methionine tRNA. Suitably, the reference RNA is a mt-tRNA, optionally a methionine mt-tRNA. Suitably, c_{ref} is determined by a method described herein. Suitably, c_{wt}, cₘᵤₜ, and c_{ref} are determined by a method described herein.

The relative quantity of wild-type RNA molecules and/or relative quantity of mutant or aberrantly-modified RNA molecules may be used to detect, diagnose and/or assess the clinical severity of a RNA-associated disease. A lower quantity of wild-type RNA molecules and/or a higher quantity of mutant or aberrantly-modified RNA molecules may be associated with increased clinical severity of a RNA-associated disease.

In some embodiments, the method of detecting, diagnosing and/or assessing the clinical severity of a RNA-associated disease may further comprise (in addition to or in the absence of steps (d) and (e)):
(f) determining the concentration of wild-type RNA genes (c′_{wt}) in the sample;
(g) determining the concentration of mutant RNA genes (c′ₘᵤₜ) in the sample; and
(h) calculating the percentage DNA mutation load in the sample, c′ₘᵤₜ/(c′_{wt}+c′ₘᵤₜ).

Suitably, c′_{wt} and c′ₘᵤₜ are determined by a method described herein. Suitably, c_{wt}, cₘᵤₜ, c′_{wt} and c′ₘᵤₜ are determined by a method described herein.

The percentage DNA mutation load may be used in combination with the percentage RNA mutation load to detect, diagnose and/or assess the clinical severity of a RNA-associated disease. A higher RNA mutation load and a higher DNA mutation load may be associated with increased clinical severity of a RNA-associated disease.

In some embodiments, the method of detecting, diagnosing and/or assessing the clinical severity of a RNA-associated disease may further comprise (in addition to or in the absence of steps (d) and (e) and/or steps (f) to (h)):
(i) determining the DNA copy number.

The DNA copy number may be determined by any suitable method, for example the method described in Grady, J. P., et al., 2014. PLoS One, 9(12), p. e114462. Suitably, the DNA copy number may be the total DNA copy number or the wild-type DNA copy number.

The DNA copy number may be used in combination with any other measure described herein to detect, diagnose and/or assess the clinical severity of a RNA-associated disease.

A lower DNA copy number may be associated with increased clinical severity of a RNA-associated disease.

Any further measure (e.g. the age of the subject) may be used to detect, diagnose and/or assess the clinical severity of a RNA-associated disease. For example, it has been shown that the age of the subject is associated with the clinical severity of MELAS (J. P. Grady et al, EMBO Mol Med. 10, e8262 (2018)).

Exemplary Method of Detecting, Diagnosing and/or Assessing the Clinical Severity of MELAS

A method of detecting, diagnosing and/or assessing the clinical severity of MELAS may comprise:
(a) determining the concentration of wild-type leucine(UUR) mt-tRNAs (c_{wt}) in a sample;
(b) determining the concentration of mutant leucine(UUR) mt-tRNAs with mutation A3243G in the sample;
(c) calculating the percentage A3243G RNA mutation load, cₘᵤₜ/(c_{wt}+cₘᵤₜ) in the sample;
(d) optionally determining the concentration of a reference RNA (c_{ref}) in the sample;
(e) optionally calculating the relative quantity of wild-type leucine(UUR) mt-tRNAs in the sample, c_{wt}/c_{ref} and/or calculating the relative quantity of mutant leucine(UUR) mt-tRNAs with mutation A3243G in the sample, cₘᵤₜ/c_{ref}
(f) optionally determining the concentration of wild-type leucine(UUR) mt-tRNA genes (c′_{wt}) in the sample;
(g) optionally determining the concentration of mutant leucine(UUR) mt-tRNA genes with mutation A3243G (c′ₘᵤₜ) in the sample;
(h) optionally calculating the percentage DNA mutation load in the sample, c′ₘᵤₜ/(c′_{wt}+c′ₘᵤₜ);
(i) optionally determining the mtDNA copy number.

The sample may be obtained or obtainable from urine, muscle, blood and/or saliva. Suitably the sample is obtained or obtainable from urine. Urine is an attractive tissue for diagnostics since it is obtained in a fully non-invasive manner. Suitably the sample is obtained or obtainable from blood. Suitably, the method is used for more than one sample, optionally from more than one tissue.

The concentration of wild-type leucine(UUR) mt-tRNAs and/or the concentration of mutant leucine(UUR) mt-tRNAs may be determined by using a padlock probe, kit and/or RNA quantification system of the present invention (e.g. using a method according to the present invention).

The concentration of wild-type leucine(UUR) mt-tRNA genes and/or the concentration of mutant leucine(UUR) mt-tRNA genes may also be determined using a padlock probe (e.g. using a method described herein).

A higher RNA mutation load (and optionally one or more of: a lower quantity of wild-type leucine(UUR) mt-tRNAs and/or a higher quantity of mutant leucine(UUR) mt-tRNAs with mutation A3243G, a higher DNA mutation load, a lower mtDNA copy number, and or increased age of the subject) may be associated with increased clinical severity of MELAS.

### EXAMPLES

### Example 1—Method to Determine Mt-tRNA Mutation Load

Overview

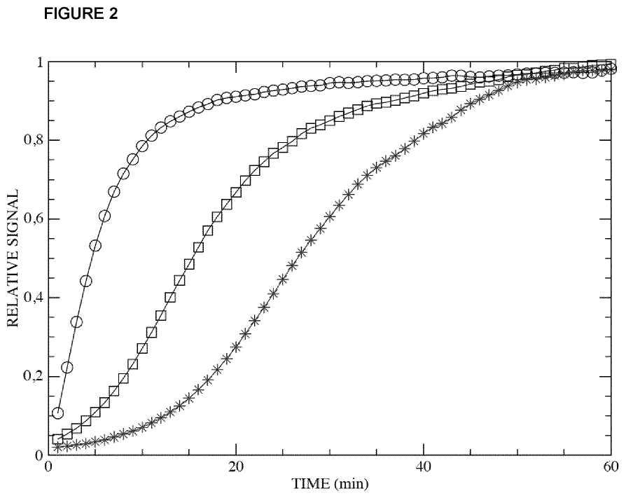

The measurement of mutations in RNA (and optionally DNA) involves four main steps.
1. Extraction of RNA (and optionally DNA).
2. Circularization of DNA oligonucleotide probes by hybridization and ligation on the RNA (and optionally DNA) target. Specificity is assured by the hybridization and the enzymatic ligation. This step is illustrated in FIG. 1.
3. Optional removal of non-circularised probes by purification using magnetic beads or specific digestion by an exonuclease cocktail
4. Detection of circularised probes. For example, hyperbranched rolling-circle amplification (HRCA) of the DNA circles and real-time fluorescence detection can be used to measure the concentrations of the different species in solution. FIG. 2 shows typical three curves obtained by our real-time HRCA measurement.

These steps are described in more detail in the protocols section below.

Results

We studied 8 samples obtained from culture of cybrid cells, 2 samples obtained from culture of skin fibroblasts and 2 samples extracted from patient blood. Two of them are controls, C1 and C8, for whom we expected 0% mutation load in both DNA and RNA. Each value of columns 3 and 4 is an average of at least two separate measurements, each measurement containing three technical replicas.

TABLE 1
Results of mutation load measurement of the MELAS mutation.

| | | | | % mut DNA by | |
|---|---|---|---|---|---|
| | | | | RFLP | |
| | | % mut | % mut | (30 | (20 |
| Type | Sample | DNA | RNA | cycles) | cycles) |
| Cybrid | C1 | 0.2 | 0.3 | 0 | — |
| Cybrid | C2 | 96.7 | 42.4 | 88 | — |
| Cybrid | C3 | 96.7 | 55.3 | 88 | — |
| Cybrid | C4 | 94.2 | 67.1 | 90 | — |
| Cybrid | C5 | 98.9 | 99.88 | 90 | 100 |
| Cybrid | C6 | 99.6 | 98.7 | 74 | 100 |
| Cybrid | C7 | 83 | 12.6 | 61 | 69 |
| Cybrid | C8 | 0.43 | 3.4 | 0 | 0 |
| Fibroblast | F1 | 12.7 | 3.2 | 6.6 | — |
| Fibroblast | F2 | 13 | 3.9 | 6.3 | — |
| Patient | P1 | 76 | 39.6 | 57 | 66 |
| Patient | P2 | 19.9 | 13.95 | 21 | 59 |

Column 3 and 4 were obtained by the approach of the present invention, for DNA and RNA, respectively. Typical statistical variations are 5% RMS in these cases. Column 5 and 6 were measured by the traditional PCR RFLP approach, using 30 and 20 PCR cycles, respectively, digestion by Apal and electrophoresis quantification with a commercial, microfluidics-based electrophoresis instrument (Agilent, Bioanalyzer 2100).

Discussion

In the method described herein, specific hybridisation and ligation of a circularisable DNA probe is performed on the target RNA and subsequently the resulting circles are amplified by hyper-branched rolling circle amplification (HRCA) (P. M. Lizardi et al, Nature Genetics 19, 225-232 (1998)). The RNA sequence required to form the DNA circle can be as short as twenty nucleotides, or less if required, which provides versatility to specifically target even short and highly structured RNA molecules.

We use our approach to quantify the well-known MELAS mutation m.3243A>G in both mitochondrial tRNA and mitochondrial DNA (mtDNA). The transfer RNA (tRNA) is short, strongly structured and exhibits some chemical modifications. We propose that measuring the mutation load of the tRNA has clinical utility. It is expected that the mutation load of the tRNA is more closely related to the cell phenotype than the mutation load of the corresponding DNA, because the tRNA is involved in protein synthesis, while mitochondrial DNA is just an intermediate. We measured a number of different samples and observed that the mutation load of the tRNA differs significantly from the DNA one. The two quantities are not just proportional, the RNA mutation load provides additional information. We propose that these measurements correlate with the clinical evolution of a patient, and propose using them as a prognostic tool and/or as an indicator for the appropriate medical treatment.

The method allows quantifying mutation rates in RNA and DNA with good precision, sensitivity and specificity, even though initially HRCA was not considered being useful for quantitative analyses (Dahl, F., et al., 2004. Proceedings of the National Academy of Sciences, 101(13), pp. 4548-4553). Our circle purification uses a biotinylated capture probe oligonucleotide that binds the RNA target and subsequent purification with streptavidin-coated paramagnetic beads. This purification of circularised probes against non-circularised probes was previously published (J H Smith et al, BioMed Res. Int. 2014, 641090).

The m.3243A>G mutation has a broad clinical expression, ranging from absence of symptoms (asymptomatic carrier) to a multi-systemic disease leading to very premature death (P. Kaufmann et al, Neurology 77, 1965-71 (2011); E. Malfati et al, Neurology 80, 100-105 (2013)). The differences relate to age of onset of the pathology, clinical severity and the specifically affected organs: skeletal muscles, eye muscles, endocrine pancreas, brain, heart (A. W. El-Hattab et al, Mol. Genet. Metab. 116, 4-12 (2015)). It is important to note that this extreme variability in the progression occurs within a same family of patients, showing the same mutation. In the most severe cases, the premature death may be caused by convulsive seizures, heart failure, renal failure or multiple organ dysfunction with lactic acidosis. The possibility of a 3243A>G mutation should also be kept in mind for diabetes, sensorineural hearing impairment, short stature or delayed maturation, exercise intolerance, and milder neurological manifestations such as migraine and learning difficulties (J. Uusimaa et al, Ann. Neurol. 62, 278-87 (2007)).

The heteroplasmy level can vary considerably from one individual to another. There is a relation between mutation load and clinical severity, which seems to be stronger if the mutation load is measured in blood, as compared to urine or muscle tissues (P. F. Chinnery et al, Brain 120, 1713-1721 (1997); S. J. Pickett et al, Ann Clin Transl Neurol. 5, 333-45 (2018)). However, sizeable inter individual variations are observed, even if complementary data is used, like copy-number of mtDNA or mutation load measured on muscle tissue. It is thus very difficult to base a reliable prognostic on heteroplasmy values obtained from mtDNA target molecules. The m.8344A>G mutation, associated with the inherited mitochondrial disease MERRF (myoclonic epilepsy with ragged-red fibres), is a further interesting case for which a correlation between the frequency of the more common clinical features and the level of mutant mtDNA was reported.

The methods herein could improve the prognostics of disease caused by m.3243A>G by measuring the mutation load on the tRNA. The measurement could be used for reliable prognostics of disease progression, which would allow adapting the treatment to the individual patient. The methods herein may also be used for determining the favourable or unfavourable effect of the transcriptional response, unveiled by the relation between the heteroplasmy of the DNA and the tRNA, as well as by the levels of the mutated and non-mutated tRNAs. These measurements could orient therapeutic research toward modulation of the transcriptional response, toward its activation or inhibition.

We performed a study of 10 blood and cell model samples carrying the m.3243A>G mutation. In all cases, the mutation load measured on RNA was lower than the one measured on the DNA, but by varying proportion (see Table 1). This RNA/DNA difference probably reflects a reduced stability of the mutated tRNA, perhaps partially compensated by an increase in mitochondrial transcription. It is possible that this reduction of mutation load is a favourable factor, reducing for instance amino acid mis-incorporation. On the other hand, it could also be unfavourable if compensatory increase of mitochondrial transcription does not occur. In the latter case the number of leucine tRNA would decrease and in turn lead to a global reduction of the mitochondrial translation.

We hypothesise that the mutation load measured on mt-tRNA, exhibits a significant, positive or negative, correlation with the severity of clinical impairment of MELAS patients. In order to evaluate the possible compensation of transcription, we propose measuring the quantity of leucine-1 tRNA in comparison to the cytosolic methionine tRNA. Met-tRNA here acts as a level indicator of the global transcriptional activity of the cell.

The methods described herein gives access to new values and in turn new combinations of values. To improve molecular diagnostics and disease prognosis for mitochondrial disorders related to a mutation in a tRNA coding gene, we propose measuring the quantities of the mutated and non-mutated forms of the mt-tRNA, the quantities of the mutated and non-mutated forms of the mt-DNA and the quantity of a reference RNA acting as indicator of the level of transcriptional activity. Our technique allows measuring these values by the same experimental approach and within the same measurement run, which provide good relative precision and facilitates quantitative comparison between values. The resulting set of data can be used in a manner that can be optimised and adjusted, leveraging results of future clinical studies.

At this stage and regarding the MELAS mutation, we can exploit (i) the heteroplasmy level of the tRNA itself, (ii) the heteroplasmy level of the tRNA in comparison with the heteroplasmy level of the DNA, (iii) the quantity of the mutated and non-mutated tRNA in comparison with the quantity of control tRNAs that are quantitative indicators of global or mitochondrial transcription activity, (iv) a combined use of (i)-(iii), possibly with a set of weighing factors to calculate a score that optimises the value of the molecular diagnostics. To achieve most reliable prognostics of disease progression and best adaption of treatment to the individual patient, it is useful to combine molecular diagnostics with other clinical diagnostic criteria. Clinical diagnostic criteria for MELAS are reported in the literature (Hirano et al, Neuromusc. Disord. 2, 125-135 (1992); Yatsuga et al, Biochim. Biophys. Acta 1820, 619-624 (2012)).

Besides providing new access to mutation frequencies of tRNA and other RNA targets that are difficult to analyse, our invention also has advantages as compared to existing technology for measuring mutation load in DNA.

First, it provides a unified platform to quantitatively address RNA and DNA, which has advantages when combining RNA and DNA results and also saves costs and space, because operational know-how, equipment and most of the consumables can be used for both RNA and DNA. Our technique also has advantages if it is compared to existing techniques to measure DNA mutation load, independent of its RNA capability. PCR Restriction Fragment Length Polymorphism (RFLP) is a traditional approach for measuring mutation load in mitochondrial DNA and is still widely used today (L. M. Scholle et al, Genes 11, 212 (2020)). There, a fragment carrying the mutation site is amplified from a DNA sample by PCR. Subsequently, a restriction endonuclease that specifically cleaves either the mutated or the non-mutated DNA is used. The number of resulting fragments, resolved by electrophoresis for instance, depends on presence/absence of the mutation.

Regarding quantitative analysis of mutation load in a mixed population of mutated and non-mutated templates, however, RFLP has a serious, well-recognized problem. During PCR heteroduplexes are formed between strands arising from the mutated and the non-mutated species. These heteroduplexes are not cleaved and thus cause bias. For RFLP analysis of the MELAS mutation with Apal, for instance, the mutation load of the m.3243A>G mutation is systematically underestimated (see Table 1). Moreover, the precise value depends on the PCR conditions, for instance the number of cycles used (also Table 1). This makes it very difficult to obtain results that can be compared from one laboratory to another; different PCR protocols and PCR machines exhibiting different temperature gradients are used. There is a possibility to circumvent the heteroduplex problem by “last-cycle hot” PCR, but this approach requires using radioactivity (Yatsuga et al, Biochim. Biophys. Acta 1820, 619-624 (2012)).

In well-equipped laboratories, DNA mutations are often analysed by next generation sequencing (NGS). The technique is powerful and can provide heteroplasmy values. However, NGS is an expensive and complex approach, the workflow before sequencing and the subsequent data analysis must be done with know-how and care, and it is difficult to give a reliable estimate for the precision of a heteroplasmy value obtained by NGS. Laboratories in rural regions or low- and middle-income countries often are not even equipped for NGS. In addition, the technique requires more input DNA than our approach, which can be a serious disadvantage when tissues with low DNA content are used, like small volume muscle biopsies, urine or buccal epithelial cell. The latter tissues are interesting, since they allow for less invasive molecular diagnostics.

If a molecular tag was used on the amplification primers, different DNA and RNA species (e.g. in the exemplified MELAS case wtDNA, mutDNA, wt-tRNA, mut-tRNA, mitochondrial methionine tRNA reference, nuclear ini methionine tRNA reference) could be amplified and detected in multiplex. Several mutations in parallel can also be quantified.

Protocols

1. Extraction

We studied 8 samples obtained from culture of cybrid cells, 2 samples obtained from culture of skin fibroblasts and 2 samples extracted from patient blood. Two of them are controls, C1 and C8, for whom we expected 0% mutation load in both DNA and RNA.

For cybrid and fibroblast samples, we used commercial extraction kits according to the supplier's protocol (Qiagen RNeasy for RNA and DNeasy for DNA). For DNA and RNA extraction from blood we used PAXgene Blood DNA tubes and PAXgene blood RNA tubes according to the commercial protocol, respectively. Other extraction procedures can be employed. Extracted nucleic acids can be transported on dry ice and stored at −80° C.

2. Circularization of DNA Oligonucleotide Probes and Ligation

a) DNA Targets

In a 5 μL reaction volume containing Hifi Taq ligase buffer (1×), we added two restriction enzymes (BsII (2.5 Units) and MboII (1.25 Units) to the purified DNA targets (typically 1 μL of DNA at a concentration of 40 ng/μL). The reaction was incubated at 37° C. for 15 minutes, at 55° C. for 15 minutes followed by inactivation for 20 minutes at 80° C. Then, we added the ligation mix (5 μL), which contains 0.1 pmol of wt (or mut) circularizable probes, 0.1 pmol of capture probes and 0.25 μL of Hifi Taq DNA ligase. After a hybridization step (ramp between 97° C. and 37° C., 5° C. decrement every 2 minutes), we incubated the tubes for 30 minutes at 56.6° C. We transferred the sample to a DNA Lobind plate (Eppendorf) and performed magnetic bead-based purification, as described below.

b) RNA Targets

In a 5 μL reaction volume containing Splint R ligase buffer (1×), we added 0.1 pmol of wt or mut circularizable probes, 0.1 pmol of capture probes and 1 μL of RNA template (typically 40-60 ng). After a hybridization step (ramp between 97° C. and 37° C., 5° C. decrement every 2 minutes), we added 0.25 μL of SplintR ligase (6.25 units) then we incubated the tubes for 45 minutes (or less) at 37° C. We transferred the sample to a DNA Lobind plate (Eppendorf) and performed magnetic bead-based purification, as described below.

3. Purification after Circle Ligation

a) Purification Using Magnetic Beads
Add 2 μL of 10% Tween 20 and 10 μL of Streptavidin coated Dynabeads (M 270, Dynal, dilution prepared according to supplier's protocol)
Mix well with pipet
Incubate 10-15 min on orbital shaker at RT
Set tubes on magnet for 2 min
Discard solution while on the magnet
Add 50 μL of Wash buffer
Mix well with pipet
Repeat Wash step twice
Elute in 18 μL dH20
Mix well with pipet
Incubate 5 min at 80° C.
Set tubes on magnet for 2 min
Take supernatant for subsequent amplification step

b) Removal of Non-Circularised Probes by Specific Exonuclease Digestion

We directly added to each tube 2 μL of Isothermal Amplification buffer 10×, water (to get a total 20 μL volume) and Exonuclease cocktail (Exo I (10U), Lambda Exo (1.25 U), and T5 Exo (2.5 U)). Each tube was incubated for 90 min at 37° C. followed by 20 min at 80° C. Finally, we added the HRCA mastermix to the product of exonuclease restriction and performed HRCA in triplicates as described elsewhere.

4. Amplification and Real-Time Detection

Next, we added 34 μL of HRCA mix (1× Isothermal amplification buffer supplemented with 18 nmol dNTP, 18 pmoles forward primer, 18 pmoles reverse primer, 3.6 μL DMSO, 1.8 μL SybrGREEN I 10×, and 0.9 μL Bst DNA polymerase Warmstart) to tubes.

The tubes are distributed in triplicates on a white Twintec plate (Eppendorf). After sealing the plate and a short centrifugation, we incubated at 65° C. for 1 hour in a commercial real-time PCR machine (Eppendorf Mastercycler). SybrGREEN fluorescence is recorded during this incubation.

5. Quantification

On DNA, we typically measure two concentrations.

| | |
|---|---|
| Concentration of non-mutated species | C_{wt} |
| Concentration of mutated species | Cₘᵤₜ |
| Mutation load is defined by | Cₘᵤₜ/(C_{wt} + Cₘᵤₜ) |

On RNA, we typically measure three concentrations.

| | |
|---|---|
| Concentration of non-mutated RNA species | C_{wt} |
| Concentration of mutated RNA species | Cₘᵤₜ |
| Concentration of a control RNA, indicator of | C_{ref} |
| transcription activity | |
| Mutation load | Cₘᵤₜ/(C_{wt} + Cₘᵤₜ) |
| Relative quantity of the non-mutated species | C_{wt}/C_{ref} |
| Relative quantity of the mutated species | Cₘᵤₜ/C_{ref} |

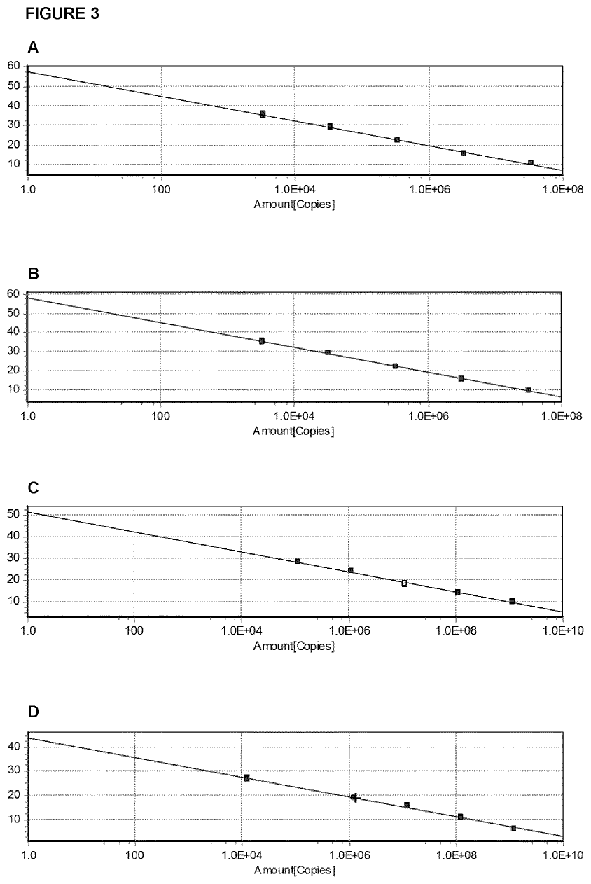

We quantify the number of target molecules in each sample by comparing the amplification curve of the sample to reference curves obtained with a series of standards submitted to the same ligation-purification-HRCA run (FIGS. 3A-D). The reference target molecules used to obtain the standard curves were prepared before the study, using protocols given below.

a) For DNA

For one of the wt DNA measurements of sample C3, we measured a characteristic time cₜ of 22.65 min. Comparison of this value to FIG. 3A leads to an amount c_{wt} of 3.53E5 copies.

For the corresponding mut DNA measurement of sample C3, we measured a characteristic time cₜ of 13.21 min. Comparison of this value to FIG. 3B leads to an amount cₘᵤₜ of 8.81E6 copies.

Restricting, for the purpose of illustration, the calculation to this particular pair of measurements, the DNA mutation load of this sample would be cₘᵤₜ/(c_{wt}+cₘᵤₜ)=0.96, i.e. 96%.

b) For RNA

For one of the wt RNA measurements of sample C3, we measured a characteristic time cₜ of 23.1 min. Comparison of this value to FIG. 3C leads to an amount c_{wt} of 1.45E6 copies.

For the corresponding mut RNA measurement of sample C3, we measured a characteristic time cₜ of 18.75 min. Comparison of this value to FIG. 3D leads to an amount cₘᵤₜ of 1.53E6 copies.

Restricting, for the purpose of illustration, the calculation to this particular pair of measurements, the RNA mutation load of this sample would be cₘᵤₜ/(c_{wt}+cₘᵤₜ)=0.51, i.e. 51%.

6. Preparation of Reference Targets

We prepared in vitro transcribed tRNA (wt and mut) to serve as template in the standard curves. In vitro transcribed tRNA is convenient and appropriate for the purpose of this invention.

We designed two forward oligonucleotides (one for wt variant of Leu and one for mutant). The forward oligonucleotides are composed of the promoter sequence of T7 RNA polymerase and the 40 first nucleotides of the Leu UUR tRNA. A reverse oligonucleotide is designed to overlap the forward primer so that its 3′ end (last 21 bases) can hybridize the 3′end of the forward oligonucleotide.

We synthetized DNA molecules (wt and mut) by using a MuIV-RT (Revertaid) polymerase to transform this template into a full double stranded molecule. First, we prepared a 40 μL hybridization mix as follows: 960 pmoles of Forward oligo added to 960 pmoles of Reverse oligo and 0.8 μL of Tris Buffer 1M (pH 7.5). We incubated the mix at 95° C. for 2 min and at RT for 3 min.

Then, we prepared a 160 μL polymerase mix as follows: we mixed 40 μL of 5×MuIV-RT Buffer, 10 mM dNTP, 4 μL of MuIV-RT polymerase and 108 μL of dH₂0. Finally, we added the Mastermix to the oligos and incubated the mix at 37° C. for 40 minutes. The resulting product is purified by using a phenol chloroform extraction.

This synthesized DNA that we sometimes call tDNA is used as reference in the DNA measurements. It can serve as DNA reference template provided there are no aborted products (this can be controlled, for instance by classical gel electrophoresis or a commercial, microfluidics-based electrophoresis instrument). Optionally one can perform a PCR of this DNA to assure that there is a single species.

To prepare the reference RNA, we used T7 polymerase to transcribe the reference DNA. First, we prepared a 250 μL transcription Mastermix as follows: we mixed 4×21 μL of 100 mM NTPs, 52.5 μL of 100 mM GMP, 210 μL of 5×T7 Transcription buffer, 26.25 μL of Ribolock, 63 μL of T7 polymerase and dH₂O. Then, we prepared a 250 μL template mix containing 20 μg of tDNA prepared at the previous step. Finally, we added the transcription Mastermix to the template mix and incubated overnight at 37° C.

We then proceeded to the isolation and purification of the in vitro transcribed tRNA. We prepared 20% denaturing polyacrylamide gels (8M urea). We loaded the transcription products and after appropriate migration time, we either used UV shadowing or Sybr Gold staining to visualize the band of interest. The bands corresponding to the tRNA are cut using a scalpel and placed in a 15 mL tube. We delicately crushed the gel slices using a pipet tip and added crush and soak buffer until the gel pieces are covered by the buffer. Then, the tubes are placed on a rotating shaker overnight at 4° C.

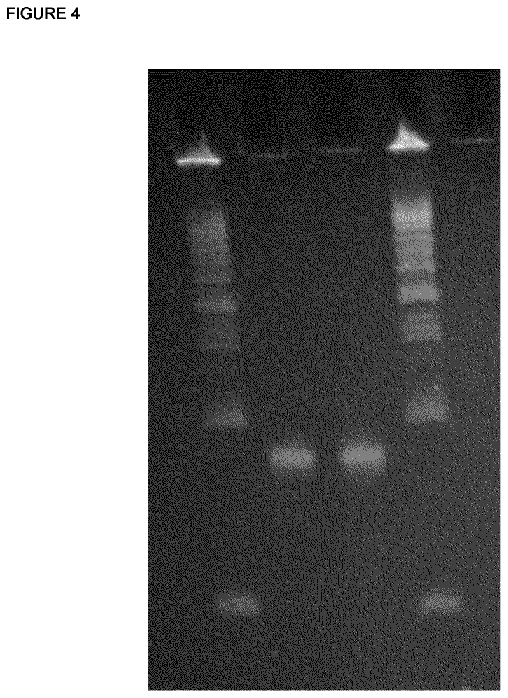

Then, we spin down the tubes with 7000 RPM for 10 minutes at 4° C. The solutions are then filtered through a syringe and its 0.45 m filter. We finally performed ethanol precipitation and eluted the purified tRNA in dH₂0 and thus obtained the desired in vitro transcribed tRNAs. The expected size of 78 nt was controlled as well as the fact that it was indeed RNA and not DNA (by digestion with Rnase and Exo I) (FIG. 4).

7. Sequences

In all target sequences: (i) the site complementary to the first terminal region of the corresponding circularizable probe is shown underlined; (ii) the site complementary to the second terminal region of the corresponding circularizable probe is shown in italics and underlined; (iii) the mutation site (if present) is shown in bold.

In all circularizable probes sequences: (i) the first terminal region is shown underlined; (ii) the first primer binding site is underlined in bold; (iii) the second primer binding site is shown in italics and underlined in bold; (iv) the second terminal region is shown in italics and underlined; (v) the mutation site (if present) is shown in bold.

In all forward oligos for preparing reference samples, the region complementary to the corresponding tRNA is underlined. In all reverse oligos for preparing reference samples, the overlap with forward oligo is underlined.

a) WT Leu(UUR) Mt-DNA Sequences

Target sequence
AAGAAGAGGAATTGAACCTCTGACTGTAAAGTTTTAAGTTTTATGCGATTACCGG
GCTCTGCCATCTTAACAAACCCTGTTCTTGGGTGG
Circularizable probe
GCCCGGTAATCGCATAAAACTTAAAACTAAA [symbol] [symbol]
AAATTAAGATGGCAGA
Forward primer
GAGTCCTGCTACAAGATTTCA
Reverse primer
TGGACAGGCAAGGAATACAGG
Capture probe
Biot 5′ ACA-GTC-AGA-GGT-TCA-ATT-CCT-CTT-CT 3′spacer C3

b) MUT Leu(UUR) Mt-DNA Sequences

Target sequence
AAGAAGAGGAATTGAACCTCTGACTGTAAAGTTTTAAGTTTTATGCGATTACCGG
GCCCTGCCATCTTAACAAACCCTGTTCTTGGGTGG
Circularizable probe
GCCCGGTAATCGCATAAAACTTAAAACTAAA [symbol] [symbol] [symbol] [symbol]
AAAATTAAGATGGCAG
Forward primer
ACACTTCCAAACTCTCTCAATC
Reverse primer
CTGTCCAGGGATCTGCTCTT
Capture probe
Biot 5′ ACA-GTC-AGA-GGT-TCA-ATT-CCT-CTT-CT 3′spacer C3

c) WT Leu(UUR) Mt-RNA Sequences

Target sequence
GUUAAGAUGGCAGAGCCCGGUAAUCGCAUAAAACUUAAAACUUUACAGUCAG
AGGUUCAAUUCCUCUUCUUAACACCA
Circularizable probe
CTGCCATCTTAAC [symbol] [symbol] [symbol] [symbol]
TTACCGGGCT
Forward primer
GAAGTCTTCGTTCTTTACCTCACCA
Reverse primer
TTATCATTATCTACGAGCGGACGA
Capture probe
Biotin 5′TGG-TGT-TAA-GAA-GAG-GAA-TTG-AAC-CTC-T 3′ Spacer C3
Forward oligo for preparing reference samples
TAATACGACTCACTATAGGTTAAGATGGCAGAGCCCGGTAATCGCATAAAACTT
AAAAC
Reverse oligo for preparing reference samples
TGGTGTTAAGAAGAGGAATTGAACCTCTGACTGTAAAGTTTTAAGTTTTATGCGA
TTA

d) MUT Leu(UUR) Mt-RNA Sequences

Target sequence
GUUAAGAUGGCAGGGCCCGGUAAUCGCAUAAAACUUAAAACUUUACAGUCAG
AGGUUCAAUUCCUCUUCUUAACACCA
Circularizable probe
CTGCCATCTTAAC [symbol] [symbol] [symbol] [symbol]
TTACCGGGCC
Forward primer
TGGTCAGCTCCTCCCCTACA
Reverse primer
CACTTATCATTATCTTCGCACAGACG
Capture probe
Biotin 5′TGG-TGT-TAA-GAA-GAG-GAA-TTG-AAC-CTC-T 3′ Spacer C3
Forward oligo for preparing reference samples
TAATACGACTCACTATAGGTTAAGATGGCAGGGCCCGGTAATCGCATAAAACTT
AAAAC
Reverse oligo for preparing reference samples
TGGTGTTAAGAAGAGGAATTGAACCTCTGACTGTAAAGTTTTAAGTTTTATGCGA
TTA

e) Nuclear Ini Methionine tRNA Sequences

tDNA target sequence
AGCAGAGTGGCGCAGCGGAAGCGTGCTGGGCCCATAACCCAGAGGTCGATGG
ATCGAAACCATCCTCTGCTACCA
tRNA target sequence
AGCAGAGUGGCGCAGCGGAAGCGUGCUGGGCCCAUAACCCAGAGGUCGAUG
GAUCGAAACCAUCCUCUGCUACCA
Circularizable probe
ATGGGCCCAGCACGCTTC [symbol] [symbol] [symbol] [symbol]
CATCGACCTCTGGGTT
Forward primer
GAGTCCTGCTACAAGATTTCA
Reverse primer
TGACAGGCAAGGAATACAGG
Capture probe
Biotin 5′CTGCGCCACTCTGCT 3′Spacer C3
Forward oligo for preparing reference samples
TAATACGACTCACTATAGAGCAGAGTGGCGCAGCGGAAGCGTGCTGGGCCCAT
AACCCAG
Reverse oligo for preparing reference samples
TGGTAGCAGAGGATGGTTTCGATCCATCGACCTCTGGGTTATGGGCCCAGCAC
GCTTC

f) Mitochondrial Methionine tRNA Sequences

tDNA target sequence
AGTAAGGTCAGCTAAATAAGCTATCGGGCCCATACCCCGAAAATGTTGGTTATA
CCCTTCCCGTACTACCA
tRNA target sequence
AGUAAGGUCAGCUAAAUAAGCUAUCGGGCCCAUACCCCGAAAAUGUUGGUUA
UACCCUUCCCGUACUACCA
Circularizable probe
AGCTTATTTAGCTGACCTTACT [symbol] [symbol] [symbol] [symbol]
ATTTTCGGGGTATGGGCCCGAT
Forward primer
ATGACAAGGCACGATCCATAC
Reverse primer
CGTGGACGCCAGAAAATTAAG
Capture probe
Biotin TAG-TAC-GGG-AAG-GGT-AT SpacerC3
Forward oligo for preparing reference samples
TAATACGACTCACTATAGAGTAAGGTCAGCTAAATAAGCTATCGGGCCCATACC
CCGAAA
Reverse oligo for preparing reference samples
TGGTAGTACGGGAAGGGTATAACCAACATTTTCGGGGTATGGGCCCGATA

All publications mentioned in the above specification are herein incorporated by reference. Various modifications and variations of the disclosed methods, cells, compositions and uses of the invention will be apparent to the skilled person without departing from the scope and spirit of the invention. Although the invention has been disclosed in connection with specific preferred embodiments, it should be understood that the invention as claimed should not be unduly limited to such specific embodiments. Indeed, various modifications of the disclosed modes for carrying out the invention, which are obvious to the skilled person are intended to be within the scope of the following claims.

1. A padlock probe comprising terminal regions complementary to a tRNA, wherein a terminal region is complementary to a region of the tRNA that is associated with a pathogenic mutation.

2. The padlock probe according to claim 1, wherein the pathogenic mutation is a pathogenic single-nucleotide variant.

3. The padlock probe according to claim 1 or claim 2, wherein (i) the tRNA is a leucine(UUR) mt-tRNA and the pathogenic mutation is A3243G or T3271C; or (ii) the tRNA is a lysine mt-tRNA and the pathogenic mutation is A8344G.

4. The padlock probe according to any preceding claim, the region of the tRNA that is associated with a pathogenic mutation comprises or consists of:
(i) a fragment of the nucleotide sequence
(SEQ ID NO: 25)
GUUAAGAUGGCAGGGCCCGGUAAUCGCAUAAAACUUAAAACUUUACAGU
CAGAGGUUCAAUUCCUCUUCUUAACACCA,
optionally wherein the fragment comprises nucleotide 14 of SEQ ID NO: 25; or
(ii) a fragment of the nucleotide sequence
(SEQ ID NO: 23)
GUUAAGAUGGCAGAGCCCGGUAAUCGCAUAAAACUUAAAACUUUACAGU
CAGAGGUUCAAUUCCUCUUCUUAACACCA,
optionally wherein the fragment comprises nucleotide 14 of SEQ ID NO: 23.

5. The padlock probe according to any preceding claim, wherein a terminal region comprises or consist of the nucleotide sequence TTACCGGGCC (SEQ ID NO: 56) or TTACCGGGCT (SEQ ID NO: 58), or fragments thereof.

6. The padlock probe according to any preceding claim, wherein the terminal regions comprise or consist of the nucleotide sequences CTGCCATCTTAAC and TTACCGGGCC (SEQ ID NOs: 55 and 56) or CTGCCATCTTAAC and TTACCGGGCT (SEQ ID NOs: 57 and 58), or fragments thereof.

7. The padlock probe according to any preceding claim, wherein the padlock probe comprises or consists of a nucleotide sequence with at least 70%, at least 80%, at least 85%, at least 90%, at least 95%, at least 98%, or at least 99% sequence identity to
(SEQ ID NO: 63)
CTGCCATCTTAACTGTAGGGGAGGAGCTGACCACACTTATCATTATCTT
CGCACAGACGTTACCGGGCC
or
(SEQ ID NO: 64)
CTGCCATCTTAACTGGTGAGGTAAAGAACGAAGACTTCTTATCATTATC
TACGAGCGGACGATTACCGGGCT

8. The padlock probe according to any preceding claim, wherein the padlock probe comprises or consists of
(SEQ ID NO: 63)
CTGCCATCTTAACTGTAGGGGAGGAGCTGACCACACTTATCATTATCTT
CGCACAGACGTTACCGGGCC
or
(SEQ ID NO: 64)
CTGCCATCTTAACTGGTGAGGTAAAGAACGAAGACTTCTTATCATTATC
TACGAGCGGACGATTACCGGGCT

9. The padlock probe according to any preceding claim, wherein the region of the tRNA that is associated with a pathogenic mutation comprises the pathogenic mutation.

10. A padlock probe comprising terminal regions complementary to a tRNA, wherein a terminal region is complementary to a region of the tRNA that is associated with a modified nucleotide.

11. The padlock probe according to claim 10, wherein the modified nucleotide is selected from: N2,N2-dimethyl guanosine (m²₂G), 5-methylcytosine (m⁵C), 7-methylguanosine (m⁷G), 5-methoxycarbonylmethyl-2-thiouridine (mcm⁵s²U), 5-methyl uridine (m⁵U), 1-methylguanosine (m¹G), 5-methoxycarbonylmethyluridine (mcm⁵U), 2-methylthio-N6-threonyl carbamoyladenosine (ms²t⁶A), 5-taurinomethyluridine (τm⁵U), 5-taurinomethyl-2-thiouridine (τm⁵s²U), and 2-thiouridine (s²U).

12. The padlock probe according to claim 10 or claim 11, wherein the modified nucleotide is 5-taurinomethyluridine (τm⁵U) or 5-taurinomethyl-2-thiouridine (τm⁵s²U).

13. The padlock probe according to any one of claims 10 to 12, wherein the region of the tRNA that is associated with a modified nucleotide comprises an aberrantly modified nucleotide, optionally wherein the aberrant nucleotide modification is the absence of the nucleotide modification.

14. The padlock probe according to any one of claims 10 to 13, wherein aberrant modification of the modified nucleotide is pathological.

15. The padlock probe according to any one of claims 10 to 14, wherein (i) the tRNA is a leucine(UUR) mt-tRNA and the modified nucleotide is τm⁵U or (ii) the tRNA is a lysine mt-tRNA and the modified nucleotide is τm⁵s²U.

16. The padlock probe according to any one of claims 10 to 15, wherein the region of the tRNA that is associated with a modified nucleotide comprises or consists of a fragment of
(SEQ ID NO: 25)
GUUAAGAUGGCAGGGCCCGGUAAUCGCAUAAAACUUAAAACUUUACAGU
CAGAGGUUCAAUUCCUCUUCUUAACACCA
optionally wherein the fragment comprises nucleotide 36 of SEQ ID NO: 25.

17. A padlock probe comprising terminal regions complementary to a tRNA comprising a 3′ ligated oligonucleotide, wherein a terminal region is complementary to a region of the tRNA that comprises at least part of the 3′ ligated oligonucleotide.

18. The padlock probe according to any preceding claim, wherein the tRNA is a mitochondrial tRNA (mt-tRNA).

19. The padlock probe according to any preceding claim, wherein the tRNA is a leucine(UUR) mt-tRNA, a lysine mt-tRNA, a methionine mt-tRNA, a tryptophan mt-tRNA, a aspartate mt-tRNA, an isoleucine mt-tRNA, a glycine mt-tRNA, an arginine mt-tRNA, a histidine mt-tRNA, a serine(AGY) mt-tRNA, a leucine(CUN) mt-tRNA, a threonine mt-tRNA, a phenylalanine mt-tRNA, a valine mt-tRNA, a glutamine mt-tRNA, an alanine mt-tRNA, an asparagine mt-tRNA, a cysteine mt-tRNA, a tyrosine mt-tRNA, a serine(UCN) mt-tRNA, a glutamate mt-tRNA, or a proline mt-tRNA.

20. The padlock probe according to any preceding claim, wherein the tRNA is a leucine(UUR) mt-tRNA, a lysine mt-tRNA, a histidine mt-tRNA, a leucine(CUN) mt-tRNA, a phenylalanine mt-tRNA, a valine mt-tRNA, a glutamine mt-tRNA, a serine(UCN) mt-tRNA, or a proline mt-tRNA.

21. The padlock probe according to any preceding claim, wherein the tRNA is a leucine(UUR) mt-tRNA or a lysine mt-tRNA, optionally wherein the tRNA is a leucine(UUR) mt-tRNA.

22. The padlock probe according to any preceding claim, wherein the region of the tRNA that is associated with a pathogenic mutation, associated with a modified nucleotide or comprises at least part of the 3′ ligated oligonucleotide is 5-30 nucleotides in length, optionally 5-15 nucleotides in length.

23. The padlock probe according to any preceding claim, wherein the terminal regions are each 5-30 nucleotides in length, optionally 5-15 nucleotides in length.

24. The padlock probe according to any preceding claim, wherein the terminal regions in total are 15-50 nucleotides in length, optionally 20-30 nucleotides in length.

25. The padlock probe according to any preceding claim, wherein the terminal regions are complementary to adjacent regions of the RNA.

26. The padlock probe according to claim any one of claims 1-25, wherein the terminal regions are complementary to non-adjacent regions of the RNA, optionally wherein the non-adjacent regions are separated by 6 or fewer nucleotides, 5 or fewer nucleotides, 4 or fewer nucleotides, 3 or fewer nucleotides, 2 or fewer nucleotides, or one nucleotide.

27. The padlock probe according to any preceding claim, wherein the padlock probe is 50-200 nucleotides in length, 50-150 nucleotides in length, 50-100 nucleotides in length, or 70-100 nucleotides in length.

28. The padlock probe according to any preceding claim, wherein the padlock probe comprises one or more primer binding sites, optionally wherein the padlock probe comprises a forward primer site and a reverse primer site.

29. The padlock probe according to any preceding claim, wherein the padlock probe comprises from 5′ to 3′: a first terminal region; a first primer binding site; a second primer binding site; and a second terminal region; optionally wherein the first primer binding site is a forward primer site and the second primer binding site is a reverse primer binding site, or the first primer binding site is a reverse primer site and the second primer binding site is a forward primer binding site.

30. The padlock probe according to claim 28 or claim 29, wherein the primer binding sites are 10-30 nucleotides in length.

31. The padlock probe according to any preceding claim, wherein the padlock probe comprises a tag or barcode sequence.

32. A kit or composition comprising one or more padlock probes according to any preceding claim.

33. The kit or composition according to claim 32, wherein the one or more padlock probes comprise or consist of:
(i) a first padlock probe comprising terminal regions complementary to a tRNA, wherein a terminal region is complementary to a region of the tRNA that is associated with a pathogenic mutation and the region does not comprise the pathogenic mutation; and
(ii) a second padlock probe comprising terminal regions complementary to said tRNA, wherein a terminal region is complementary to a region of the tRNA that comprises the pathogenic mutation.

34. The kit or composition according to claim 33, wherein the kit or composition further comprises:
(iii) a third padlock probe comprising terminal regions complementary to a gene encoding said tRNA, wherein a terminal region is complementary to a region of the gene that is associated with a pathogenic mutation and the region does not comprise the pathogenic mutation; and
(iv) a fourth padlock probe comprising terminal regions complementary to the gene encoding said tRNA, wherein a terminal region is complementary to a region of the gene that comprises the pathogenic mutation.

35. The kit or composition according to any one of claims 32 to 34, wherein the kit or composition further comprises a fifth padlock probe comprising terminal regions complementary to a reference nuclear tRNA, optionally a nuclear methionine tRNA.

36. The kit or composition according to any one of claims 32 to 35, wherein the kit or composition further comprises a sixth padlock probe comprising terminal regions complementary to a reference mt-tRNA, optionally a methionine mt-tRNA.

37. The kit or composition according to any one of claims 32 to 36, further comprising one or more primers, optionally one forward primer and one reverse primer.

38. The kit or composition according to claim 37, wherein the one or more primers are complementary to one or more primer binding sites on the one or more padlock probes.

39. The kit or composition according to any one of claims 32 to 38, further comprising one or more capture probes.

40. The kit or composition according to any one of claims 32 to 39, further comprising:
(i) a DNA ligase, optionally a Splint R ligase; and/or
(ii) a DNA ligase buffer, optionally a Splint R ligase buffer.

41. The kit or composition according to any one of claims 32 to 40, further comprising:
(i) an amplification buffer;
(ii) a deoxynucleoside triphosphate (dNTP) mix;
(iii) a DNA polymerase; and/or
(iv) a DNA dye.

42. A method of detecting a tRNA:
(a) providing a sample comprising one or more tRNAs;
(b) hybridising one or more padlock probes defined according to any one of claims 1 to 31 to the one or more tRNAs to obtain one or more hybridised padlock probes;
(c) circularising the one or more hybridised padlock probes to obtain one or more circularised padlock probes;
(d) optionally purifying the one or more circularised padlock probes;
(e) amplifying the one or more circularised padlock probesto obtain amplified padlock probes; and
(f) detecting the amplified padlock probes.

43. The method according to claim 42, wherein the tRNAs are mitochondrial tRNAs (mt-tRNAs).

44. The method according to claim 42 or claim 43, wherein the one or more tRNAs are leucine(UUR) mt-tRNAs, lysine mt-tRNAs, methionine mt-TRNAs, tryptophan mt-tRNAs, aspartate mt-tRNAs, isoleucine mt-tRNAs, glycine mt-tRNAs, arginine mt-tRNAs, histidine mt-tRNAs, serine(AGY) mt-tRNAs, leucine(CUN) mt-tRNAs, threonine mt-tRNAs, phenylalanine mt-tRNAs, valine mt-tRNAs, glutamine mt-tRNAs, alanine mt-tRNAs, asparagine mt-tRNAs, cysteine mt-tRNAs, tyrosine mt-tRNAs, serine(UCN) mt-tRNAs, glutamate mt-tRNAs, and/or proline mt-tRNAs.

45. The method according to any one of claims 42 to 44, wherein the one or more tRNAs are leucine(UUR) mt-tRNAs, lysine mt-tRNAs, histidine mt-tRNAs, leucine(CUN) mt-tRNAs, phenylalanine mt-tRNAs, valine mt-tRNAs, glutamine mt-tRNAs, serine(UCN) mt-tRNAs, or proline mt-tRNAs.

46. The method according to any one of claims 42 to 45, wherein the one or more tRNAs are leucine(UUR) mt-tRNAs and/or lysine mt-tRNAs, optionally wherein the one or more tRNAs are leucine(UUR) mt-tRNAs.

47. The method according to any one of claims 43 to 46, wherein the tRNAs comprises a 3′ ligated oligonucleotide and/or the method further comprises a step of ligating an oligonucleotide to the 3′ end of the tRNAs prior to step (b).

48. The method according to any one of claims 42 to 47, wherein the sample is an RNA sample.

49. The method according to any one of claims 42 to 48, wherein the sample is obtained or obtainable from urine, muscle and/or blood.

50. The method according to any one of claims 42 to 49, wherein the method further comprises a step of extracting, purifying and/or isolating the sample from urine, muscle and/or blood.

51. The method according to any one of claims 42 to 50, wherein step (c) is performed using a DNA ligase, optionally a Splint R ligase.

52. The method according to any one of claims 42 to 51, wherein step (d) is performed by magnetic bead-based purification and/or exonuclease digestion of non-circularised padlock probes.

53. The method according to any one of claims 42 to 52, wherein the amplification in step (e) is performed by rolling circle amplification (RCA), optionally hyperbranched RCA (HRCA).

54. The method according to any one of claims 42 to 53, wherein step (f) is performed by detecting an increase in fluorescence, optionally the increase in fluorescence is detected in real-time.

55. The method according to any one of claims 42 to 54, further comprising a step (g) of quantifying the number of tRNAs.

56. The method according to claim 55, wherein the number of tRNAs is quantified relative to a reference sample.

57. A method of detecting, diagnosing and/or assessing the clinical severity of a tRNA-associated disease comprising:
(a) determining the concentration of wild-type tRNAs (c_{wt});
(b) determining the concentration of mutant or aberrantly-modified tRNAs (cₘᵤₜ); and
(c) calculating the percentage tRNA mutation load or aberrant-modification load, cₘᵤₜ/(c_{wt}+cₘᵤₜ).

58. The method according to claim 57, wherein the tRNA-associated disease is a mt-tRNA-associated disease and the tRNAs are mt-tRNAs,

59. The method according to claim 58, wherein the mt-tRNA-associated disease is mitochondrial encephalopathy, lactic acidosis, and stroke-like episodes (MELAS) syndrome or myoclonic epilepsy with ragged red fibers syndrome (MERRF) syndrome, optionally wherein the mt-tRNA-associated disease is MELAS syndrome.

60. The method according to any one of claims 57 to 59, wherein the concentration of mutant tRNAs is determined, and wherein, optionally: (i) the tRNAs are leucine(UUR) mt-tRNAs and the mutation is m.3243A>G; or (ii) the tRNAs are lysine mt-tRNAs and the mutation is m.8344A>G.

61. The method according to any one of claims 57 to 59, wherein the concentration of aberrantly-modified tRNAs is determined, and wherein, optionally: (i) the tRNAs are leucine(UUR) mt-tRNAs and the modified nucleotide is τm⁵U or (ii) the tRNAs are lysine mt-tRNAs and the modified nucleotide is τm⁵s²U.

62. The method according to any one of claims 57 to 61, further comprising:
(d) determining the concentration of a reference tRNA (c_{ref}); and
(e) calculating the relative quantity of wild-type tRNA molecules, c_{wt}/c_{ref} and/or calculating the relative quantity of mutant or aberrantly-modified tRNA molecules, cₘᵤₜ/c_{ref}.

63. The method according to claim 62, wherein the reference tRNA is a nuclear tRNA, optionally a nuclear methionine tRNA, or a mt-tRNA, optionally a methionine mt-tRNA.

64. The method according to any one of claims 57 to 63, wherein one or more of c_{wt}, cₘᵤₜ, and c_{ref} is determined by a method according to any one of claims 42 to 56.

65. The method according to any one of claims 57 to 64, further comprising:
(f) determining the concentration of wild-type tRNA genes (c′_{wt});
(g) determining the concentration of mutant tRNA genes (c′ₘᵤₜ); and
(h) calculating the percentage DNA mutation load, c′ₘᵤₜ/(c′_{wt}+c′ₘᵤₜ).

66. Use of a padlock probe for detecting, diagnosing and/or assessing the clinical severity of a tRNA-associated disease.

67. The use according to claim 66, wherein the tRNA-associated disease is a mt-tRNA-associated disease

68. The use according to claim 67, wherein the mt-tRNA-associated disease is mitochondrial encephalopathy, lactic acidosis, and stroke-like episodes (MELAS) syndrome or myoclonic epilepsy with ragged red fibers syndrome (MERRF) syndrome, optionally wherein the mt-tRNA-associated disease is MELAS syndrome.

69. The use according to any one of claims 66-68, wherein the padlock probe is defined according to any one of claims 1 to 31.

70. Use of a padlock probe for detecting and/or quantifying tRNA amino-acid charging.


SEQUENCE LISTING (103 sequences)

SEQ ID NO: 1 - RNA, 75 nt, Artificial Sequence
  Synthetic polynucleotide
guuaagauggcagagcccgguaaucgcauaaaacuuaaaacuuuacagucagagguucaauuccucuucuuaaca

SEQ ID NO: 2 - RNA, 70 nt, Artificial Sequence
  Synthetic polynucleotide
cacuguaaagcuaacuuagcauuaaccuuuuaaguuaaagauuaagagaaccaacaccucuuuacaguga

SEQ ID NO: 3 - RNA, 68 nt, Artificial Sequence
  Synthetic polynucleotide
aguaaggucagcuaaauaagcuaucgggcccauaccccgaaaauguugguuauacccuucccguacua

SEQ ID NO: 4 - RNA, 68 nt, Artificial Sequence
  Synthetic polynucleotide
agaaauuuagguuaaauacagaccaagagccuucaaagcccucaguaaguugcaauacuuaauuucug

SEQ ID NO: 5 - RNA, 68 nt, Artificial Sequence
  Synthetic polynucleotide
aagguauuagaaaaaccauuucauaacuuugucaaaguuaaauuauaggcuaaauccuauauaucuua

SEQ ID NO: 6 - RNA, 69 nt, Artificial Sequence
  Synthetic polynucleotide
agaaauaugucugauaaaagaguuacuuugauagaguaaauaauaggagcuuaaacccccuuauuucua

SEQ ID NO: 7 - RNA, 68 nt, Artificial Sequence
  Synthetic polynucleotide
acucuuuuaguauaaauaguaccguuaacuuccaauuaacuaguuuugacaacauucaaaaaagagua

SEQ ID NO: 8 - RNA, 65 nt, Artificial Sequence
  Synthetic polynucleotide
ugguauauaguuuaaacaaaacgaaugauuucgacucauuaaauuaugauaaucauauuuaccaa

SEQ ID NO: 9 - RNA, 69 nt, Artificial Sequence
  Synthetic polynucleotide
guaaauauaguuuaaccaaaacaucagauugugaaucugacaacagaggcuuacgaccccuuauuuacc

SEQ ID NO: 10 - RNA, 59 nt, Artificial Sequence
  Synthetic polynucleotide
gagaaagcucacaagaacugcuaacucaugcccccaugucuaacaacauggcuuucuca

SEQ ID NO: 11 - RNA, 71 nt, Artificial Sequence
  Synthetic polynucleotide
acuuuuaaaggauaacagcuauccauuggucuuaggccccaaaaauuuuggugcaacuccaaauaaaagua

SEQ ID NO: 12 - RNA, 66 nt, Artificial Sequence
  Synthetic polynucleotide
guccuuguaguauaaacuaauacaccagucuuguaaaccggagaugaaaaccuuuuuccaaggaca

SEQ ID NO: 13 - RNA, 71 nt, Artificial Sequence
  Synthetic polynucleotide
guuuauguagcuuaccuccucaaagcaauacacugaaaauguuuagacgggcucacaucaccccauaaaca

SEQ ID NO: 14 - RNA, 69 nt, Artificial Sequence
  Synthetic polynucleotide
cagaguguagcuuaacacaaagcacccaacuuacacuuaggagauuucaacuuaacuugaccgcucuga

SEQ ID NO: 15 - RNA, 72 nt, Artificial Sequence
  Synthetic polynucleotide
uaggauggggugugauagguggcacggagaauuuuggauucucagggauggguucgauucucauaguccuag

SEQ ID NO: 16 - RNA, 69 nt, Artificial Sequence
  Synthetic polynucleotide
aagggcuuagcuuaauuaaaguggcugauuugcguucaguugaugcagagugggguuuugcaguccuua

SEQ ID NO: 17 - RNA, 73 nt, Artificial Sequence
  Synthetic polynucleotide
uagauugaagccaguugauuagggugcuuagcuguuaacuaaguguuuguggguuuaagucccauuggucuag

SEQ ID NO: 18 - RNA, 66 nt, Artificial Sequence
  Synthetic polynucleotide
agcuccgaggugauuuucauauugaauugcaaauucgaagaagcagcuucaaaccugccggggcuu

SEQ ID NO: 19 - RNA, 66 nt, Artificial Sequence
  Synthetic polynucleotide
gguaaaauggcugagugaagcauuggacuguaaaucuaaagacagggguuaggccucuuuuuacca

SEQ ID NO: 20 - RNA, 69 nt, Artificial Sequence
  Synthetic polynucleotide
gaaaaagucauggaggccaugggguuggcuugaaaccagcuuugggggguucgauuccuuccuuuuuug

SEQ ID NO: 21 - RNA, 69 nt, Artificial Sequence
  Synthetic polynucleotide
guucuuguaguugaaauacaacgaugguuuuucauaucauuggucgugguuguaguccgugcgagaaua

SEQ ID NO: 22 - RNA, 68 nt, Artificial Sequence
  Synthetic polynucleotide
cagagaauaguuuaaauuagaaucuuagcuuugggugcuaaugguggaguuaaagacuuuuucucuga

SEQ ID NO: 23 - RNA, 78 nt, Artificial Sequence
  Synthetic polynucleotide
guuaagauggcagagcccgguaaucgcauaaaacuuaaaacuuuacagucagagguucaauuccucuucuuaacacca

SEQ ID NO: 24 - RNA, 73 nt, Artificial Sequence
  Synthetic polynucleotide
cacuguaaagcuaacuuagcauuaaccuuuuaaguuaaagauuaagagaaccaacaccucuuuacagugacca

SEQ ID NO: 25 - RNA, 78 nt, Artificial Sequence
  Synthetic polynucleotide
guuaagauggcagggcccgguaaucgcauaaaacuuaaaacuuuacagucagagguucaauuccucuucuuaacacca

SEQ ID NO: 26 - RNA, 75 nt, Artificial Sequence
  Synthetic polynucleotide
guuaagauggcagagcccgguaaucgcauaaaacuuaaaaccuuacagucagagguucaauuccucuucuuaaca

SEQ ID NO: 27 - RNA, 73 nt, Artificial Sequence
  Synthetic polynucleotide
cacuguaaagcuaacuuagcauuaaccuuuuaaguuaaagauuaagagagccaacaccucuuuacagugacca

SEQ ID NO: 28 - DNA, 75 bp, Artificial Sequence
  Synthetic polynucleotide
tgttaagaagaggaattgaacctctgactgtaaagttttaagttttatgcgattaccgggctctgccatcttaac

SEQ ID NO: 29 - DNA, 70 bp, Artificial Sequence
  Synthetic polynucleotide
tcactgtaaagaggtgttggttctcttaatctttaacttaaaaggttaatgctaagttagctttacagtg

SEQ ID NO: 30 - DNA, 68 bp, Artificial Sequence
  Synthetic polynucleotide
tagtacgggaagggtataaccaacattttcggggtatgggcccgatagcttatttagctgaccttact

SEQ ID NO: 31 - DNA, 68 bp, Artificial Sequence
  Synthetic polynucleotide
cagaaattaagtattgcaacttactgagggctttgaaggctcttggtctgtatttaacctaaatttct

SEQ ID NO: 32 - DNA, 68 bp, Artificial Sequence
  Synthetic polynucleotide
taagatatataggatttagcctataatttaactttgacaaagttatgaaatggtttttctaatacctt

SEQ ID NO: 33 - DNA, 69 bp, Artificial Sequence
  Synthetic polynucleotide
tagaaataagggggtttaagctcctattatttactctatcaaagtaactcttttatcagacatatttct

SEQ ID NO: 34 - DNA, 68 bp, Artificial Sequence
  Synthetic polynucleotide
tactcttttttgaatgttgtcaaaactagttaattggaagttaacggtactatttatactaaaagagt

SEQ ID NO: 35 - DNA, 65 bp, Artificial Sequence
  Synthetic polynucleotide
ttggtaaatatgattatcataatttaatgagtcgaaatcattcgttttgtttaaactatatacca

SEQ ID NO: 36 - DNA, 69 bp, Artificial Sequence
  Synthetic polynucleotide
ggtaaataaggggtcgtaagcctctgttgtcagattcacaatctgatgttttggttaaactatatttac

SEQ ID NO: 37 - DNA, 59 bp, Artificial Sequence
  Synthetic polynucleotide
tgagaaagccatgttgttagacatgggggcatgagttagcagttcttgtgagctttctc

SEQ ID NO: 38 - DNA, 71 bp, Artificial Sequence
  Synthetic polynucleotide
tacttttatttggagttgcaccaaaatttttggggcctaagaccaatggatagctgttatcctttaaaagt

SEQ ID NO: 39 - DNA, 66 bp, Artificial Sequence
  Synthetic polynucleotide
tgtccttggaaaaaggttttcatctccggtttacaagactggtgtattagtttatactacaaggac

SEQ ID NO: 40 - DNA, 71 bp, Artificial Sequence
  Synthetic polynucleotide
tgtttatggggtgatgtgagcccgtctaaacattttcagtgtattgctttgaggaggtaagctacataaac

SEQ ID NO: 41 - DNA, 69 bp, Artificial Sequence
  Synthetic polynucleotide
tcagagcggtcaagttaagttgaaatctcctaagtgtaagttgggtgctttgtgttaagctacactctg

SEQ ID NO: 42 - DNA, 72 bp, Artificial Sequence
  Synthetic polynucleotide
ctaggactatgagaatcgaacccatccctgagaatccaaaattctccgtgccacctatcacaccccatccta

SEQ ID NO: 43 - DNA, 69 bp, Artificial Sequence
  Synthetic polynucleotide
taaggactgcaaaaccccactctgcatcaactgaacgcaaatcagccactttaattaagctaagccctt

SEQ ID NO: 44 - DNA, 73 bp, Artificial Sequence
  Synthetic polynucleotide
ctagaccaatgggacttaaacccacaaacacttagttaacagctaagcaccctaatcaactggcttcaatcta

SEQ ID NO: 45 - DNA, 66 bp, Artificial Sequence
  Synthetic polynucleotide
aagccccggcaggtttgaagctgcttcttcgaatttgcaattcaatatgaaaatcacctcggagct

SEQ ID NO: 46 - DNA, 66 bp, Artificial Sequence
  Synthetic polynucleotide
tggtaaaaagaggcctaacccctgtctttagatttacagtccaatgcttcactcagccattttacc

SEQ ID NO: 47 - DNA, 69 bp, Artificial Sequence
  Synthetic polynucleotide
caaaaaaggaaggaatcgaaccccccaaagctggtttcaagccaaccccatggcctccatgactttttc

SEQ ID NO: 48 - DNA, 69 bp, Artificial Sequence
  Synthetic polynucleotide
tattctcgcacggactacaaccacgaccaatgatatgaaaaaccatcgttgtatttcaactacaagaac

SEQ ID NO: 49 - DNA, 68 bp, Artificial Sequence
  Synthetic polynucleotide
tcagagaaaaagtctttaactccaccattagcacccaaagctaagattctaatttaaactattctctg

SEQ ID NO: 50 - DNA, 78 bp, Artificial Sequence
  Synthetic polynucleotide
tggtgttaagaagaggaattgaacctctgactgtaaagttttaagttttatgcgattaccgggccctgccatcttaac

SEQ ID NO: 51 - DNA, 73 bp, Artificial Sequence
  Synthetic polynucleotide
tggtcactgtaaagaggtgttggttctcttaatctttaacttaaaaggttaatgctaagttagctttacagtg

SEQ ID NO: 52 - DNA, 78 bp, Artificial Sequence; identical to SEQ ID NO: 50
  Synthetic polynucleotide

SEQ ID NO: 53 - DNA, 75 bp, Artificial Sequence
  Synthetic polynucleotide
tgttaagaagaggaattgaacctctgactgtaaggttttaagttttatgcgattaccgggctctgccatcttaac

SEQ ID NO: 54 - DNA, 73 bp, Artificial Sequence
  Synthetic polynucleotide
tggtcactgtaaagaggtgttggctctcttaatctttaacttaaaaggttaatgctaagttagctttacagtg

SEQ ID NO: 55 - DNA, 13 bp, Artificial Sequence
  Synthetic polynucleotide
ctgccatcttaac

SEQ ID NO: 56 - DNA, 10 bp, Artificial Sequence
  Synthetic polynucleotide
ttaccgggcc

SEQ ID NO: 57 - DNA, 13 bp, Artificial Sequence
  Synthetic polynucleotide
ctgccatcttaac

SEQ ID NO: 58 - DNA, 10 bp, Artificial Sequence
  Synthetic polynucleotide
ttaccgggct

SEQ ID NO: 59 - DNA, 18 bp, Artificial Sequence
  Synthetic polynucleotide
atgggcccagcacgcttc

SEQ ID NO: 60 - DNA, 16 bp, Artificial Sequence
  Synthetic polynucleotide
catcgacctctgggtt

SEQ ID NO: 61 - DNA, 22 bp, Artificial Sequence
  Synthetic polynucleotide
agcttatttagctgaccttact

SEQ ID NO: 62 - DNA, 22 bp, Artificial Sequence
  Synthetic polynucleotide
attttcggggtatgggcccgat

SEQ ID NO: 63 - DNA, 69 bp, Artificial Sequence
  Synthetic polynucleotide
ctgccatcttaactgtaggggaggagctgaccacacttatcattatcttcgcacagacgttaccgggcc

SEQ ID NO: 64 - DNA, 72 bp, Artificial Sequence
  Synthetic polynucleotide
ctgccatcttaactggtgaggtaaagaacgaagacttcttatcattatctacgagcggacgattaccgggct

SEQ ID NO: 65 - DNA, 76 bp, Artificial Sequence
  Synthetic polynucleotide
atgggcccagcacgcttctgaaatcttgtagcaggactcgtgacaggcaaggaatacaggcatcgacctctgggtt

SEQ ID NO: 66 - DNA, 86 bp, Artificial Sequence
  Synthetic polynucleotide
agcttatttagctgaccttactgtatggatcgtgccttgtcatcgtggacgccagaaaattaagattttcggggtatgggcccgat

SEQ ID NO: 67 - DNA, 28 bp, Artificial Sequence
  Synthetic polynucleotide
gcccggtaatcgcataaaacttaaaact

SEQ ID NO: 68 - DNA, 12 bp, Artificial Sequence
  Synthetic polynucleotide
ttaagatggcag

SEQ ID NO: 69 - DNA, 28 bp, Artificial Sequence
  Synthetic polynucleotide
gcccggtaatcgcataaaacttaaaact

SEQ ID NO: 70 - DNA, 13 bp, Artificial Sequence
  Synthetic polynucleotide
ttaagatggcaga

SEQ ID NO: 71 - DNA, 89 bp, Artificial Sequence
  Synthetic polynucleotide
gcccggtaatcgcataaaacttaaaactaaagattgagagagtttggaagtgtctgtccagggatctgctcttaaaattaagatggcag

SEQ ID NO: 72 - DNA, 89 bp, Artificial Sequence
  Synthetic polynucleotide
gcccggtaatcgcataaaacttaaaactaaatgaaatcttgtagcaggactctggacaggcaaggaatacaggaaattaagatggcaga

SEQ ID NO: 73 - DNA, 20 bp, Artificial Sequence
  Synthetic polynucleotide
tggtcagctcctcccctaca

SEQ ID NO: 74 - DNA, 26 bp, Artificial Sequence
  Synthetic polynucleotide
cacttatcattatcttcgcacagacg

SEQ ID NO: 75 - DNA, 25 bp, Artificial Sequence
  Synthetic polynucleotide
gaagtcttcgttctttacctcacca

SEQ ID NO: 76 - DNA, 24 bp, Artificial Sequence
  Synthetic polynucleotide
ttatcattatctacgagcggacga

SEQ ID NO: 77 - DNA, 21 bp, Artificial Sequence
  Synthetic polynucleotide
gagtcctgctacaagatttca

SEQ ID NO: 78 - DNA, 20 bp, Artificial Sequence
  Synthetic polynucleotide
tgacaggcaaggaatacagg

SEQ ID NO: 79 - DNA, 21 bp, Artificial Sequence
  Synthetic polynucleotide
atgacaaggcacgatccatac

SEQ ID NO: 80 - DNA, 21 bp, Artificial Sequence
  Synthetic polynucleotide
cgtggacgccagaaaattaag

SEQ ID NO: 81 - DNA, 28 bp, Artificial Sequence
  Synthetic polynucleotide
  misc_feature (1)..(1): 5′ Biotin
  misc_feature (28)..(28): 3′ Spacer C3
tggtgttaagaagaggaattgaacctct

SEQ ID NO: 82 - DNA, 28 bp, Artificial Sequence
  Synthetic polynucleotide
  misc_feature (1)..(1): 5′ Biotin
  misc_feature (28)..(28): 3′ Spacer C3
tggtgttaagaagaggaattgaacctct

SEQ ID NO: 83 - DNA, 15 bp, Artificial Sequence
  Synthetic polynucleotide
  misc_feature (1)..(1): 5′ Biotin
  misc_feature (15)..(15): 3′ Spacer C3
ctgcgccactctgct

SEQ ID NO: 84 - DNA, 17 bp, Artificial Sequence
  Synthetic polynucleotide
  misc_feature (1)..(1): Biotin
  misc_feature (17)..(17): Spacer C3
tagtacgggaagggtat

SEQ ID NO: 85 - protein, 298 aa, Artificial Sequence
  Exemplary Splint R ligase
MAITKPLLAATLENIEDVQFPCLATPKIDGIRSVKQTQMLSRTFKPIRNSVMNRLLTELLPEGSDGEISIEGATFQDTTSAVMTGHKMYNAKFSYYWFDYVTDDPLKKYIDRVEDMKNYITVHPHILEHAQVKIIPLIPVEINNITELLQYERDVLSKGFEGVMIRKPDGKYKFGRSTLKEGILLKMKQFKDAEATIISMTALFKNTNTKTKDNFGYSKRSTHKSGKVEEDVMGSIEVDYDGVVFSIGTGFDADQRRDFWQNKESYIGKMVKFKYFEMGSKDCPRFPVFIGIRHEEDR

SEQ ID NO: 86 - DNA, 22 bp, Artificial Sequence
  Synthetic polynucleotide
acacttccaaactctctcaatc

SEQ ID NO: 87 - DNA, 20 bp, Artificial Sequence
  Synthetic polynucleotide
ctgtccagggatctgctctt

SEQ ID NO: 88 - DNA, 21 bp, Artificial Sequence
  Synthetic polynucleotide
gagtcctgctacaagatttca

SEQ ID NO: 89 - DNA, 21 bp, Artificial Sequence
  Synthetic polynucleotide
tggacaggcaaggaatacagg

SEQ ID NO: 90 - DNA, 26 bp, Artificial Sequence
  Synthetic polynucleotide
  misc_feature (1)..(1): 5′ Biotin
  misc_feature (26)..(26): 3′ Spacer C3
acagtcagaggttcaattcctcttct

SEQ ID NO: 91 - DNA, 90 bp, Artificial Sequence
  Synthetic polynucleotide
aagaagaggaattgaacctctgactgtaaagttttaagttttatgcgattaccgggctctgccatcttaacaaaccctgttcttgggtgg

SEQ ID NO: 92 - DNA, 90 bp, Artificial Sequence
  Synthetic polynucleotide
aagaagaggaattgaacctctgactgtaaagttttaagttttatgcgattaccgggccctgccatcttaacaaaccctgttcttgggtgg

SEQ ID NO: 93 - DNA, 59 bp, Artificial Sequence
  Synthetic polynucleotide
taatacgactcactataggttaagatggcagagcccggtaatcgcataaaacttaaaac

SEQ ID NO: 94 - DNA, 58 bp, Artificial Sequence
  Synthetic polynucleotide
tggtgttaagaagaggaattgaacctctgactgtaaagttttaagttttatgcgatta

SEQ ID NO: 95 - DNA, 59 bp, Artificial Sequence
  Synthetic polynucleotide
taatacgactcactataggttaagatggcagggcccggtaatcgcataaaacttaaaac

SEQ ID NO: 96 - DNA, 75 bp, Artificial Sequence
  Synthetic polynucleotide
agcagagtggcgcagcggaagcgtgctgggcccataacccagaggtcgatggatcgaaaccatcctctgctacca

SEQ ID NO: 97 - RNA, 75 nt, Artificial Sequence
  Synthetic polynucleotide
agcagaguggcgcagcggaagcgugcugggcccauaacccagaggucgauggaucgaaaccauccucugcuacca

SEQ ID NO: 98 - DNA, 60 bp, Artificial Sequence
  Synthetic polynucleotide
taatacgactcactatagagcagagtggcgcagcggaagcgtgctgggcccataacccag

SEQ ID NO: 99 - DNA, 58 bp, Artificial Sequence
  Synthetic polynucleotide
tggtagcagaggatggtttcgatccatcgacctctgggttatgggcccagcacgcttc

SEQ ID NO: 100 - DNA, 71 bp, Artificial Sequence
  Synthetic polynucleotide
agtaaggtcagctaaataagctatcgggcccataccccgaaaatgttggttatacccttcccgtactacca

SEQ ID NO: 101 - RNA, 71 nt, Artificial Sequence
  Synthetic polynucleotide
aguaaggucagcuaaauaagcuaucgggcccauaccccgaaaauguugguuauacccuucccguacuacca

SEQ ID NO: 102 - DNA, 60 bp, Artificial Sequence
  Synthetic polynucleotide
taatacgactcactatagagtaaggtcagctaaataagctatcgggcccataccccgaaa

SEQ ID NO: 103 - DNA, 50 bp, Artificial Sequence
  Synthetic polynucleotide
tggtagtacgggaagggtataaccaacattttcggggtatgggcccgata